SEQUENCE LISTING (16 sequences)

SEQ ID NO: 1 - protein, 4 aa, Artificial Sequence
  Peptide
FFKY

SEQ ID NO: 2 - protein, 5 aa, Artificial Sequence
  Peptide
FFFKY

SEQ ID NO: 3 - protein, 5 aa, Artificial Sequence
  Peptide
FFGKY

SEQ ID NO: 4 - protein, 4 aa, Artificial Sequence
  Peptide
FFGK

SEQ ID NO: 5 - protein, 5 aa, Artificial Sequence
  Peptide
FFGKF

SEQ ID NO: 6 - protein, 4 aa, Artificial Sequence
  Peptide
  MISC_FEATURE (4)..(4): Xaa at position 4 is 2,6-dimethyl-L-tyrosine
FFKX

SEQ ID NO: 7 - protein, 5 aa, Artificial Sequence
  Peptide
  MISC_FEATURE (5)..(5): Xa at position 5 is 2,6-dimethyl-L-tyrosine
FFFKX

SEQ ID NO: 8 - protein, 5 aa, Artificial Sequence
  Peptide
  MISC_FEATURE (5)..(5): Xaa at position 5 is 2,6-dimethyl-L-tyrosine
FFGKX

SEQ ID NO: 9 - protein, 4 aa, Artificial Sequence
  Peptide
FFCY

SEQ ID NO: 10 - protein, 5 aa, Artificial Sequence
  Peptide
FFFCY

SEQ ID NO: 11 - protein, 5 aa, Artificial Sequence
  Peptide
FFGCY

SEQ ID NO: 12 - protein, 4 aa, Artificial Sequence
  Peptide
FFGC

SEQ ID NO: 13 - protein, 5 aa, Artificial Sequence
  Peptide
FFGCF

SEQ ID NO: 14 - protein, 4 aa, Artificial Sequence
  Peptide
  MISC_FEATURE (4)..(4): Xaa at position 4 is 2,6-dimethyl-L-tyrosine
FFCX

SEQ ID NO: 15 - protein, 5 aa, Artificial Sequence
  Peptide
  MISC_FEATURE (5)..(5): Xaa at position 5 is 2,6-dimethyl-L-tyrosine
FFFCX

SEQ ID NO: 16 - protein, 5 aa, Artificial Sequence
  Peptide
  MISC_FEATURE (5)..(5): Xaa at position 5 is 2,6-dimethyl-L-tyrosine
FFGCX

(19) United States
(12) Patent Application Publication
(10) Pub. No.: US 2024/0051992 A1
(43) Pub. Date: Feb. 15, 2024

(54) THIOPHOSPHOPEPTIDES FOR ULTRAFAST TARGETING OF THE GOLGI APPARATUS

(71) Applicant: BRANDEIS UNIVERSITY, Waltham, MA (US)

(72) Inventors: Weiyi TAN, Waltham, MA (US); Bing XU, Newton, MA (US)

(21) Appl. No.: 18/546,159

(22) PCT Filed: Feb. 11, 2022
(86) PCT No.: PCT/US2022/016151

§ 371 Date: Aug. 11, 2023

Related U.S. Application Data
(60) Provisional application No. 63/148,412, filed on Feb. 11, 2021.

(51) Int. Cl. C07K 5/072; C07K 5/087; C07K 5/107; A61K 9/107; A61P 35/00; G01N 33/58

(52) U.S. Cl. CPC C07K 5/06121; C07K 5/0812; C07K 5/1016; A61K 9/1075; A61P 35/00; G01N 33/582; A61K 38/00

(57) ABSTRACT

Disclosed are peptides having the structure according to formula (Ia) or (Ib) or (II): wherein NH-Q-C(O) is a peptide chain optionally comprising an amino acid residue having a sidechain covalently bonded to Z₂; Z₁ is a moiety comprising an aromatic group, a therapeutic agent, a fluorophore, or a nanoparticle; Z₂ is H, a therapeutic agent, a fluorophore, or a nanoparticle; and J, if present (as in Ib or II), is a linker between the peptide chain and the thiophosphate group or thioester group. Also disclosed are pharmaceutical compositions that contain a peptide, as well as dephosphorylated peptides of the invention, which may take one or more forms include a nanofiber or a supramolecular hydrogel. Use of the peptides for delivering a therapeutic agent or drug moiety into the Golgi apparatus, imaging a cell, treating a patient having a cancerous condition, treating a patient having Alzheimer's or Parkinson's disease are also disclosed.

This application claims the priority benefit of U.S. Provisional Patent Application Ser. No. 63/148,412, filed Feb. 11, 2021, which is hereby incorporated by reference in its entirety.

This invention was made with government support under CA142746 and CA252364 awarded by National Institutes of Health, and DMR-2011846 awarded by the National Science Foundation. The government has certain rights in the invention.

### FIELD OF THE INVENTION

The present invention relates to thiophosphorylated peptide, which are capable of enzymatically induced self-assembly to form larger structures, and are capable of targeted delivery to the Golgi apparatus.

### BACKGROUND OF THE INVENTION

Golgi apparatus (GA), a stack of flattened membrane-enclosed disks that are dynamically regulated during cell cycles in mammalian cells, is considered as the “heart” of intracellular transportation (Kulkarni-Gosavi et al., “Form and Function of the Golgi Apparatus: Scaffolds, Cytoskeleton and Signalling,” FEBS. Lett. 593:2289-2305 (2019); Lee et al., “Bi-directional Protein Transport Between the ER and Golgi,” Annu. Rev. Cell. Dev. Biol. 20:87-123 (2004)). Increasing numbers of studies have revealed that Golgi is a hub for different signaling pathways that drive the survival and migration of cancer cells (Bivona et al., “Phospholipase Cy Activates Ras on the Golgi Apparatus by Means of RasGRP1,” Nature 424:694-698 (2003); Farber-Katz et al., “DNA Damage Triggers Golgi Dispersal Via DNA-PK and GOLPH3,” Cell 156:413-427 (2014)). Although Golgi is emerging as an important target for cancer therapy, there are, however, few approaches for targeting Golgi (Armstrong et al., “Manno-epi-cyclophellitols Enable Activity-Based Protein Profiling of Human α-Mannosidases and Discovery of New Golgi Mannosidase II Inhibitors,” J. Am. Chem. Soc. 142:13021-13029 (2020); Van Den Elsen et al., “Structure of Golgi a-mannosidase II: A Target for Inhibition of Growth and Metastasis of Cancer Cells,” Embo. J. 20:3008-3017 (2001)). While Golgi mannosidase II inhibitors are able to inhibit cancer cells, the selectivity (Dennis et al., “Growth Inhibition of Human Melanoma Tumor Xenografts in Athymic Nude Mice by Swainsonine,” Cancer Res. 50:1867-72 (1990)) or efficacy (Armstrong et al., “Manno-epi-cyclophellitols Enable Activity-Based Protein Profiling of Human α-Mannosidases and Discovery of New Golgi Mannosidase II Inhibitors,” J. Am. Chem. Soc. 142:13021-13029 (2020)) of the inhibitors remains to be improved. In addition, several studies reported the imaging of Golgi, including the commercial dyes for staining Golgi (van Echten-Deckert, et al., “1-Methylthiodihydroceramide, A Novel Analog of Dihydroceramide, Stimulates Sphinganine Degradation Resulting in Decreased De Novo Sphingolipid Biosynthesis,” J. Biol. Chem. 273:1184-91 (1998)), a smart “off—on” fluorescence probe for imaging the Golgi in cancer cells (Zhang et al., “An Off-on COX-2-specific Fluorescent Probe: Targeting the Golgi Apparatus of Cancer Cells,” J. Am. Chem. Soc. 135″11663-11669 (2013)), and carbon quantum dots localizing at Golgi (Li et al., “Chiral Nanoprobes for Targeting and Long-term Imaging of the Golgi Apparatus. Chem. Sci. 8:6829-6835 (2017)). These imaging agents, however, require 30 minutes (Zhang et al., “An Off-on COX-2-specific Fluorescent Probe: Targeting the Golgi Apparatus of Cancer Cells,” J. Am. Chem. Soc. 135″11663-11669 (2013)) or longer incubation time (Zhang et al., “An Off-on COX-2-specific Fluorescent Probe: Targeting the Golgi Apparatus of Cancer Cells,” J. Am. Chem. Soc. 135″11663-11669 (2013)) or pretreatment (van Echten-Deckert, et al., “1-Methylthiodihydroceramide, A Novel Analog of Dihydroceramide, Stimulates Sphinganine Degradation Resulting in Decreased De Novo Sphingolipid Biosynthesis,” J. Biol. Chem. 273:1184-91 (1998)) and they have yet to lead to an approach for selectively inhibiting the cancer cells. Thus, there is an unmet need of targeting Golgi, particularly to inhibit cancer cells.

The present invention is directed to overcoming these and other deficiencies in the art.

### SUMMARY OF THE INVENTION

A first aspect of the invention relates to a peptide having the structure according to formula (Ia) or (Ib):

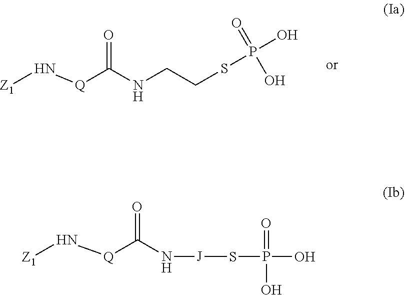

wherein,
NH—Q-C(O) is a peptide chain containing from 2 to 20 amino acids and including a plurality of aromatic amino acids, and the peptide chain optionally comprises a lysine, histidine, or arginine residue having a sidechain covalently bonded to Z₂;
Z₁ is a moiety comprising an aromatic group, a therapeutic agent, a fluorophore, or a nanoparticle;
Z₂ is H, a therapeutic agent, a fluorophore, or a nanoparticle; and
J, if present (as in Ib), is a linker between the —NH— group covalently attached to the C-terminal end of the peptide chain and the thiophosphate group.

A second aspect of the invention relates to a peptide having the structure according to formula (II):

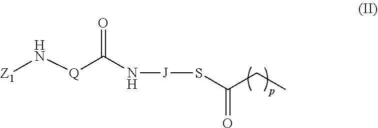

wherein,
NH-Q-C(O) is a peptide chain containing from 2 to 20 amino acids and including a plurality of aromatic amino acids, and the peptide chain optionally comprises a lysine, histidine, or arginine residue having a sidechain covalently bonded to Z₂;
Z₁ is a moiety comprising an aromatic group, a therapeutic agent, a fluorophore, or a nanoparticle;
Z₂ is H, a therapeutic agent, a fluorophore, or a nanoparticle;
p is an integer from 0 to 9; and
J is a linker between the —NH— group covalently attached to the C-terminal end of the peptide chain and the thioester (—SC(O)(CH)ₚCH₃) group.

A third aspect of the invention relates to a pharmaceutical composition that includes a peptide according to the first or second aspect in an aqueous medium. In certain embodiments, the peptides present in the pharmaceutical composition form micelle structures and a therapeutic agent is encapsulated within the micelle structures.

A fourth aspect relates to dephosphorylated peptides according to the first aspect or deesterified peptides according to the second aspect, which may take one or more forms.

According to one embodiment, a nanofiber is formed in an aqueous medium and includes self-assembled, dephosphorylated forms of the peptide according to the first aspect or deesterified peptides according to the second aspect.

According to another embodiment, a supramolecular hydrogel is formed in an aqueous medium and includes a self-assembled, dephosphorylated form of the peptide according to the first aspect or a self-assembled, deesterified form of the peptide according to the second aspect.

A fifth aspect of the invention relates to a method of delivering a therapeutic agent into the Golgi apparatus. This method includes the steps of encapsulating a therapeutic agent within a micelle structure of a pharmaceutical composition according to the third aspect; and contacting a cell with the pharmaceutical composition, whereby micelle structures are taken up by the cell and targeted to the Golgi apparatus within the cell.

A sixth aspect of the invention relates to a method of delivering a drug moiety into the Golgi apparatus. This method includes the steps of providing a peptide according to the first or second aspect, wherein Z₁ or Z₂ is a drug moiety (e.g., a therapeutic agent), or a composition according to the third aspect; and contacting a cell with the peptide or the composition, whereby the peptide, or micelle structures formed by the peptide, is taken up by the cell and targeted to the Golgi apparatus within the cell.

A seventh aspect of the invention relates to a method of treating a patient having a cancerous condition. This method includes the step of administering a pharmaceutical composition according to the third aspect to a patient having a cancerous condition, wherein said administering is effective to inhibit cancer cell survival.

An eighth aspect of the invention relates to a method of treating a patient having Alzheimer's or Parkinson's disease. This method includes the step of administering a pharmaceutical composition according to the third aspect to a patient having a Alzheimer's or Parkinson's disease, wherein said administering is effective to treat symptoms of disease.

A ninth aspect of the invention relates to a method of imaging a cell. This method includes the steps of providing a peptide according to the first or second aspect, wherein Z₁ or Z₂ is a fluorophore, or a composition comprising the peptide; contacting a cell with the peptide or the composition, whereby the peptide, or micelle structures formed by the peptide, are taken up by the cell and targeted to the Golgi apparatus within the cell; and obtaining an image of the cell, whereby the Golgi apparatus is identified by fluorescence from the fluorophore.

The accompanying examples of enzymatic noncovalent synthesis (“ENS”) (He et al., “Enzymatic Noncovalent Synthesis,” Chem. Rev. 120:9994-10078 (2020), which is hereby incorporated by reference in its entirety) were facilitated by an oxygen atom of the phosphoester bond in a phosphopeptide (pO1) being changed to a sulfur atom to make pS1 for fast enzymatic self-assembly. These studies presented here show that pS1 undergoes rapid dephosphorylation catalyzed by alkaline phosphatase (“ALP”) to form S1 that assembles. Unexpectedly, treating HeLa cells with pS1 shows that S1 instantly accumulates at Golgi of the HeLa cells at the concentration as low as 500 nM. Such an enzymatic accumulation of Golgi (FIG. 1) is proportional to both the concentration of pS1 and the time of incubation. Similar rapid enzymatic accumulation also takes places in the Golgi of several other cells (e.g., Saos2, SJSA1, OVSAHO, HCC1937, and HEK293). Unlike pS1, the parent phosphopeptide, pO1, taking longer time for dephosphorylation than that of pS1, requires hours for cellular uptake and largely remains in endosomes. These results indicate that rapid dephosphorylation of the thiophosphate group and the resulting thiol group are critical for instantly targeting Golgi. Based on this insight, we designed pS2, a nonfluorescent analogue of pS1. Being able to undergo rapid dephosphorylation catalyzed by ALP to form S2 that exhibit a critical micelle concentration (CMC) of 9.5 μM, pS2 inhibits HeLa cells with an IC₅₀ value about 3μM, an order of magnitude more potent than that of the parent phosphopeptide. Preliminary mechanistic studies indicate that (i) the thiophosphopeptides enter cells via both caveolin-mediated endocytosis and macropinocytosis, (ii) disulfide bond formation is essential for Golgi targeting, and (iii) the level of ALP of cells contributes to the rate of the accumulation of the resulting thiopeptide assemblies at the Golgi. Providing the first case of targeting Golgi based on fast enzymatic kinetics and oxidative environment of Golgi, this work illustrates a new molecular platform for designing enzyme responsive molecules that target subcellular compartment for functions.

As discussed in the accompanying examples, the invention offers a number of advantages. First, unlike previously reported agents that require at least 30 minutes incubation time or even longer time or pretreatment, peptides of the invention only takes minutes to target and image Golgi, which is over one order of magnitude more efficient than the reported agents. Second, peptides of the invention can make a connection with proteins inside the Golgi, which means it can manipulate specific proteins or even cells, and it will have much broader applications compared with previously reported agents. Third, the design of the peptides of formula (I) is quite straightforward compared with previously reported agents, which means it is more economically beneficial. This work illustrates an efficient and effective approach to image Golgi in live cells. Fourth, the thiophosphate peptides enable delivery of inhibitors into cells. Additional examples demonstrate that similar results can be achieved using the thioesterified peptide (AcS1) according to formula (II).

### BRIEF DESCRIPTION OF THE DRAWINGS

FIG. 1 is a schematic illustration of thiophosphopeptides instantly targeting the Golgi apparatus. The structural difference between peptide pS1 (NBD-(β-Ala-f-f-NH—(CH₂)₂-thiophosphate) and pO1 (NBD-(β-Ala-f-f-NH—(CH₂)₂-phosphate) are shown.

FIGS. 2A-B are panels of CLSM images of HeLa cells stained with CellLight® Golgi-RFP after treating with pS1 for 8 minutes (FIG. 2A) and treated with pS1 for 1, 4, and 8 min (FIG. 2B). Scale bars=20 μm and [pS1]=10 μM.

FIG. 3 is a panel of CLSM images of HeLa cells treated with pS1 (10 μM, 5 μM, 2 μM) for 4 min (top), S1 (10 μM, 5 μM, 2 μM) for 30 min (middle), and pO1 (100 μM, 50 μM, 20 μM) for 4 h. Scale bars=20 μm.

FIGS. 4A-B are graphs illustrating the time-dependent mean fluorescence intensity of Golgi. In FIG. 4A, HeLa cells pretreated with PLC (phospholipase C, 0.2 U, 30 min), mβCD (5 mM, 30 min), PIC (phosphatase inhibitor cocktail set 3, 4000×, 30 min), DQB (20 μM, 30 min), respectively, and then treated with pS1 (10 μM), and the pS1 treated HeLa cells in a fresh medium. In FIG. 4B, time-dependent mean fluorescence intensity of Golgi in different cancer cell lines and non-cancer cell lines treated with pS1 (10 μM).

FIG. 5A is a graph illustrating the IC₅₀ of pS2 against HeLa cells, HEK293 cells and HS-5 cells. FIG. 5B is a CLSM image of HeLa cells treated with pS2 (10 μM, 4 h) and then stained by a tetracysteine probe, FlAsH-EDT₂. Scale bar=20 μm.

FIG. 6 is a graph illustrating the time-dependent dephosphorylation of pS1 (120 μM) and pO1 (120 μM) treated with ALP.

FIG. 7 is a graph illustrating CMC determinations of pS1 and S1 by dynamic light scattering (DLS).

FIG. 8 is a panel of TEM images of pS1 with or without the addition of ALP for 24 h. Scale bars=100 nm.

FIG. 9 is a CLSM image of HeLa cells at 0 min immediately after the treatment of pS1 (10 μM). The Golgi of HeLa is marked by arrows. Scale bar=20 μm.

FIG. 10 is a graph illustrating time-dependent mean fluorescence intensity of HeLa cells treated with different concentrations of pS1.

FIG. 11 is a graph illustrating time-dependent mean fluorescence intensity of Golgi in HeLa cells treated with only pS1 (10 μM), and pretreated with different concentrations of CytD (0.5 μg/mL, 1.0 μg/mL) for 30 minutes and then treated with pS1 (10 μM).

FIG. 12 is a pair of CLSM images of HeLa cells treated with or without NEM (5 mM) combining with pS1 (10 μM) for 16 min. The Golgi of HeLa is marked by arrows. Scale bar=20 μm.

FIG. 13 is a graph illustrating accumulative ALP expression levels (obtained from Harmonizome Database (Rouillard et al., “The Harmonizome: A Collection of Processed Datasets Gathered to Serve and Mine Knowledge About Genes and Proteins,” Database 2016 (2016), which is hereby incorporated by reference in its entirety) and the apparent rate of the increase of the fluorescence at the Golgi of different cell lines.

FIG. 14 illustrates the structures of pS2 and S2, and the ALP catalyzed conversion of pS2 to S2.

FIG. 15 is a graph illustrating time-dependent dephosphorylation of pS2 (120 μM) and pO2 (120 μM) treated with ALP (0.1 U/mL), respectively.

FIG. 16 is a graph illustrating CMC determination of pS2 and S2 by dynamic light scattering (DLS).

FIG. 17 is a graph illustrating cytotoxicity of pS2 and pO2 against HeLa cells for 24 h and the values of IC₅₀ for both phosphopeptides.

FIG. 18 is a pair of graphs comparing the cytotoxicity of pS2 (left) and S2 (right) against HeLa cells, HEK293 cells and HS-5 cells for 24 h.

FIG. 19 [S20] is a graph illustrating cell viability of HeLa cells treated with only pS2 (10 μM), the mixture of pS2 (10 μM) with apoptosis and necroptosis inhibitors (Z-VAD-FMK, 50 μM; NAc, 1 mM; Nec-1, 50 μM) for 24 h.

FIG. 20 is a graph illustrating cell viability of HeLa cells treated with only pS2 (10 μM), the mixture of pS2 (10 μM) with ferroptosis inhibitors (DFO, 5 μM; Fer-1, 10 μM) and pyroptosis inhibitor (Disulfiram, 5 μM) for 24 h.

FIG. 21 is a panel of CLSM images showing HeLa cells with or without the pretreatment of pS2 (10 μM, 30 min) and then with or without staining by FlAsH-EDT 2 (1 μM, 1 h). Scale bar=20 μm.

FIG. 22 is a graph illustrating the percentage of AcS1 (1) and its hydrolyzed product S1 (2) in the mixture after treating AcS1 (10 μM) with enzymes (Alkaline phosphatase, ALP, 0.1 U/mL; Enterokinase, ENTK, 1 U/mL; Trypsin, 1 U/mL; Carboxylesterase, CES, 1 U/mL) for 24 h.

FIG. 23 is a panel of CLSM images of HeLa cells treated with AcS1 (10 μM) for 1, 4, 8, 12, 16, 24 min.

### DETAILED DESCRIPTION OF THE INVENTION

One aspect of the invention relates to a peptide comprising the structure according to formula (Ia) or (lb):

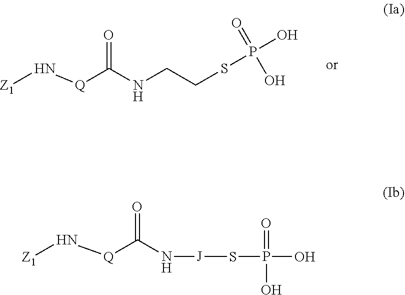

wherein,
NH-Q-C(O) is a peptide chain preferably containing from 2 to 20 amino acids and including a plurality of aromatic amino acids, and the peptide chain optionally comprises a lysine, histidine, or arginine residue having a sidechain covalently bonded to Z₂;
Z₁ is a moiety comprising an aromatic group, a therapeutic agent, a fluorophore, or a nanoparticle;
Z₂ is H, a therapeutic agent, a fluorophore, or a nanoparticle; and
J, if present (as in Ib), is a linker between the —NH— group covalently attached to the C-terminal end of the peptide chain and the thiophosphate group.

Another aspect of the invention relates to a peptide having the structure according to formula (II):

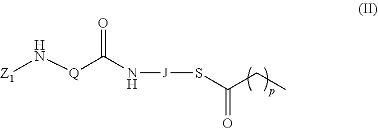

wherein,
NH-Q-C(O) is a peptide chain containing from 2 to 20 amino acids and including a plurality of aromatic amino acids, and the peptide chain optionally comprises a lysine, histidine, or arginine residue having a sidechain covalently bonded to Z₂;
Z₁ is a moiety comprising an aromatic group, a therapeutic agent, a fluorophore, or a nanoparticle;
Z₂ is H, a therapeutic agent, a fluorophore, or a nanoparticle;
p is an integer from 0 to 9 (inclusive), such as from 0 to 2, 3 to 5, or 6 to 9; and
J is a linker between the —NH— group covalently attached to the C-terminal end of the peptide chain and the thioester (—SC(O)(CH)ₚCH₃) group.

As discussed more fully below, the peptides are capable of forming micelle structures in an aqueous medium, capable of cellular uptake and targeting to the Golgi apparatus upon enzymatic dephosphorylation (i.e., hydrolytic phosphatase cleavage of thiophosphate group to form a thiol group) or deesterification (i.e., hydrolytic cleavage of the thioester group to form a thiol group). Following enzymatic cleavage, the resulting peptide is capable of self-assembling to form nanofibers and, eventually, a hydrogel in an aqueous medium.

The term “amino acid” further includes analogues, derivatives, and congeners of any specific amino acid referred to herein, as well as C-terminal or N-terminal protected amino acid derivatives (e.g., modified with an N-terminal or C-terminal protecting group). Furthermore, the term “amino acid” includes both D- and L-amino acids. Hence, an amino acid which is identified herein by its name, three letter or one letter symbol and is not identified specifically as having the D or L configuration, is understood to assume any one of the D or L configurations. For example, 2-Nal or 2-nal refer to the L and D configurations, respectively, of the analogue 3-(2-naphthyl)-alanine.

Naturally occurring amino acids are identified throughout by the conventional three-letter and/or one-letter abbreviations, corresponding to the trivial name of the amino acid, in accordance with the following list: Alanine (Ala), Arginine (Arg), Asparagine (Asn), Aspartic acid (Asp), Cysteine (Cys), Glutamic acid (Glu), Glutamine (Gln), Glycine (Gly), Histidine (His), Isoleucine (Ile), Leucine (Leu), Lysine (Lys), Methionine (Met), Phenylalanine (Phe), Proline (Pro), Serine (Ser), Threonine (Thr), Tryptophan (Trp), Tyrosine (Tyr), and Valine (Val). The abbreviations are accepted in the peptide art and are recommended by the IUPAC-IUB commission in biochemical nomenclature.

As used herein, the term “about” when used in connection with a numerical value denotes an interval of accuracy that is ±10% in certain embodiments, ±5% in other embodiments, ±2.5% in still further embodiments, and ±1% in yet another embodiment.

As used herein and in the appended claims, the singular “a”, “an” and “the” include the plural reference unless the context clearly dictates otherwise. Thus, for example, reference to a “peptide” includes a plurality of such peptides.

In some embodiments of the invention, the peptide, following phosphatase cleavage of the thiophosphate group is capable of self-assembling to form nanofibers containing the cleavage product and a hydrogel composed of such nanofibers, when present in an aqueous medium. A hydrogel may be defined as a three-dimensional, hydrophilic or amphiphilic polymeric network capable of taking up a quantity of water, typically a large quantity of water. The networks are composed of homopolymers or copolymers, are insoluble due to the presence of covalent chemical or physical (ionic, hydrophobic interactions, entanglements) crosslinks. The crosslinks provide the network structure and physical integrity. Hydrogels exhibit a thermodynamic compatibility with water that allows them to swell in aqueous media. The chains of the network are connected in such a fashion that pores exist and that a substantial fraction of these pores are of dimensions between 1 nm and 1000 nm.

As used herein and is well-known in the art, the term “hydrogel” refers to a material that comprises fibrous networks formed of water-soluble natural or synthetic polymer chains, typically (though not exclusively) containing more than 95% water, often more than 96%, 97%, 98%, or 99% water.

The term “gelling” or “gelation” means a thickening of the medium that may result in a gelatinous consistency and even in a solid, rigid consistency that does not flow under its own weight.

A “gelator” is defined herein to include a non-polymeric organic compound whose molecules can establish, between themselves, at least one physical interaction leading to a self-assembly of the molecules in a carrier fluid to form a gel. The gel may result from the formation of a network of molecular nanofibers due to the stacking or aggregation of gelator molecules. The gelator is the product of enzymatic cleavage of the peptide.

The peptide chain can have any length that is sufficient to allow for self-assembly after enzymatic cleavage. This includes peptides up to about 70 amino acids, up to about 65 amino acids, up to about 60 amino acids, up to about 55 amino acids, up to about 50 amino acids, up to about 45 amino acids, up to about 40 amino acids, up to about 35 amino acids, up to about 30 amino acids, up to about 25 amino acids, up to about 20 amino acids, up to about 15 amino acids, or up to about 10 amino acids. In certain embodiments, the peptide chain is preferably less than about 20 amino acids in length (e.g., from 2 to 20 amino acids), such as from 3 to 5 amino acids, from 5 to 7 amino acids, from 8 to 10 amino acids, from 11 to 13 amino acids, from 14 to 17 amino acids, or from 18 to 20 amino acids.

In certain embodiments of the invention, the peptide chain contains only D-amino acids. In an alternative embodiment, the peptide chain contains only L-amino acids, or a mixture of L- and D-amino acids.

To promote self-assembly, the peptide chain preferably includes aromatic amino acids, including one or more of phenylalanine, tyrosine, and tryptophan, or any derivatives thereof.

In the peptide chain, the amino acid residue to which the Z₂ moiety is covalently linked is one that has (or had, prior to such covalent linkage) a reactive sidechain. This amino acid having the reactive sidechain can be (i) one having a basic sidechain with a reactive amino group, such as Arg or Lys; or (ii) one having a nucleophilic sidechain with a reactive hydroxyl group or thiol group, such as Ser or Thr or Cys, but preferably Ser or Cys; or (iii) one having a basic sidechain with a reactive imidazole group, such as His. The amino acids having a reactive amino group will typically form a —NH—C(O)— covalent bond with the Z₂ moiety as described herein. The amino acids having a reactive hydroxyl group will typically form a —O—C(O)— covalent bond with the Z₂ moiety (see Ono et al., Bull. Chem. Soc. Japan 51(8):2401-2 404 (1978), which is hereby incorporated by reference in its entirety). The amino acids having a reactive thiol group will typically form a —S—C(O)— covalent bond with the Z₂ moiety (see Ingenito et al., JACS 121:11369-74 (1999), which is hereby incorporated by reference in its entirety).

In one embodiment, the peptide chain includes an aromatic group linked to the amino terminus of the peptide chain. The aromatic group can be any suitable single- or multi-ring aromatic moiety that facilitates self-assembly as discussed herein. Exemplary aromatic groups include, without limitation, phenylacetyl, naphthylacetyl, fluorenylacetyl, pyrenylacetyl, and cinnamoyl.

Exemplary peptides chains include, without limitation: FF, FFK, ff, ffk, FFKY (SEQ ID NO: 1), FFFKY (SEQ ID NO: 2), FFGKY (SEQ ID NO: 3), FFGK (SEQ ID NO: 4), FFGKF (SEQ ID NO: 5), ffky, fffky, ffgky, ffgk, ffgkf, FFK(Dmt) (SEQ ID NO: 6), FFFK(Dmt) (SEQ ID NO: 7), FFGK(Dmt) (SEQ ID NO: 8), ffk(dmt), fffk(dmt), ffgk(dmt), FFCY (SEQ ID NO: 9), FFFCY (SEQ ID NO: 10), FFGCY (SEQ ID NO: 11), FFGC (SEQ ID NO: 12), FFGCF (SEQ ID NO: 13), ffcy, fffcy, ffgcy, ffgc, ffgcf, FFC(Dmt) (SEQ ID NO: 14), FFFC(Dmt) (SEQ ID NO: 15), FFGC(Dmt) (SEQ ID NO: 16), ffc(dmt), fffc(dmt), ffgc(dmt), wherein Dmt is 2,6-dimethyl-L-tyrosine and dmt is 2,6-dimethyl-D-tyrosine.

The linker, J, which is present in Formula (Ib) and Formula (II) can be —(CH₂)₂—, as in Formula (Ia), or it can be another —(CH₂)ₙ— moiety where n is an integer from 3 to 10, such as 3, 4, 5, 6, 7, 8, 9, or 10; a —(CH₂)ₘ—NH—(CH₂)ₘ— moiety where each m is independently an integer from 2 to 10, such as 2, 3, 4, 5, 6, 7, 8, 9, or 10. For example, as demonstrated in the examples, solid phase synthesis can be used to react S-(2-aminoethyl) thiophosphate (or cysteamine thiophosphate) with the C-terminal carboxylic acid to form —NH(CH₂)₂S—P(O)(OH)₂. In this example, the linker, J, is —(CH₂)₂—. An alternative thiophosphate source is (3-aminopropyl)aminoethyl thiophosphate (or amifostine), which when reacted with the C-terminal carboxylic acid of the peptide will form —NH(CH₂)₃NH(CH₂)₂S—P(O)(OH)₂. In this latter example, the linker, J, is —(CH₂)₃NH(CH₂)₂—. As an alternative, in Formula (II), an S-(aminoalkyl) alkylthioate can be reacted with the C-terminal carboxylic acid to form the thioester (—SC(O)(CH)ₙCH₃) group. Exemplary S-(aminoalkyl) alkylthioates that are available include, without limitation, S-(2-aminoethyl) ethanethioate, S-(3-aminopropyl) ethanethioate, and S-(4-aminobutyl) ethanethioate, which in some embodiments are commercially available as HCl or HBr salts.

Exemplary peptides, having Z₁ and/or Z₂ groups as defined herein, include without limitation:
Naphthyl-(CH₂)—C(O)—NH—FF—C(O)—NH(CH₂)₂S—P(O)(OH)₂;
NBD-(CH₂)₂—C(O)—NH—FF—C(O)—NH(CH₂)₂S—P(O)(OH)₂;
Naphthyl-(CH₂)—C(O)—NH-ff-C(O)—NH(CH₂)₂S—P(O)(OH)₂;
NBD-(CH₂)₂—C(O)—NH-ff-C(O)—NH(CH₂)₂S—P(O)(OH)₂;
Naphthyl-(CH₂)—C(O)—NH—FFK—C(O)—NH(CH₂)₂S—P(O)(OH)₂;
NBD-(CH₂)₂—C(O)—NH—FFK—C(O)—NH(CH₂)₂S—P(O)(OH)₂;
Naphthyl-(CH₂)—C(O)—NH-ffk-C(O)—NH(CH₂)₂S—P(O)(OH)₂;
NBD-(CH₂)₂—C(O)—NH-ffk-C(O)—NH(CH₂)₂S—P(O)(OH)₂;
Naphthyl-(CH₂)—C(O)—NH—FFKY—C(O)—NH(CH₂)₂S—P(O)(OH)₂;
NBD-(CH₂)₂—C(O)—NH—FFKY—C(O)—NH(CH₂)₂S—P(O)(OH)₂;
Naphthyl-(CH₂)—C(O)—NH-ffky-C(O)—NH(CH₂)₂S—P(O)(OH)₂;
NBD-(CH₂)₂—C(O)—NH-ffky-C(O)—NH(CH₂)₂S—P(O)(OH)₂;
Naphthyl-(CH₂)—C(O)—NH—FFFKY—C(O)—NH(CH₂)2 S—P(O)(OH)₂;
NBD-(CH₂)₂—C(O)—NH—FFFKY—C(O)—NH(CH₂)₂S—P(O)(OH)₂;
Naphthyl-(CH₂)—C(O)—NH-fffky-C(O)—NH(CH₂)₂S—P(O)(OH)₂;
NBD-(CH₂)₂—C(O)—NH-fffky-C(O)—NH(CH₂)2 S—P(O)(OH)₂;
Naphthyl-(CH₂)—C(O)—NH—FFGKY—C(O)—NH(CH₂)2 S—P(O)(OH)₂;
NBD-(CH₂)₂—C(O)—NH—FFGKY—C(O)—NH(CH₂)₂S—P(O)(OH)₂;
Naphthyl-(CH₂)—C(O)—NH-ffgky-C(O)—NH(CH₂)₂S—P(O)(OH)₂;
NBD-(CH₂)₂—C(O)—NH-ffgky-C(O)—NH(CH₂)₂S—P(O)(OH)₂;
Naphthyl-(CH₂)—C(O)—NH—FFGK—C(O)—NH(CH₂)₂S—P(O)(OH)₂;
NBD-(CH₂)₂—C(O)—NH—FFGK—C(O)—NH(CH₂)₂S—P(O)(OH)₂;
Naphthyl-(CH₂)—C(O)—NH-ffgk-C(O)—NH(CH₂)₂S—P(O)(OH)₂;
NBD-(CH₂)₂—C(O)—NH-ffgk-C(O)—NH(CH₂)₂S—P(O)(OH)₂;
Naphthyl-(CH₂)—C(O)—NH—FFGKF—C(O)—NH(CH₂)2 S—P(O)(OH)₂;
NBD-(CH₂)₂—C(O)—NH—FFGKF—C(O)—NH(CH₂)₂S—P(O)(OH)₂;
Naphthyl-(CH₂)—C(O)—NH-ffgkf-C(O)—NH(CH₂)₂S—P(O)(OH)₂;
NBD-(CH₂)₂—C(O)—NH-ffgkf-C(O)—NH(CH₂)₂S—P(O)(OH)₂;
Naphthyl-(CH₂)—C(O)—NH—FFK(Dmt)-C(O)—NH(CH₂)₂S—P(O)(OH)₂;
NBD-(CH₂)₂—C(O)—NH—FFK(Dmt)-C(O)—NH(CH₂)₂S—P(O)(OH)₂;
Naphthyl-(CH₂)—C(O)—NH-ffk(dmt)-C(O)—NH(CH₂)₂S—P(O)(OH)₂;
NBD-(CH₂)₂—C (O)—NH-ffk(dmt)-C(O)—NH(CH₂)₂S—P(O)(OH)₂;
Naphthyl-(CH₂)—C(O)—NH—FFFK(Dmt)-C(O)—NH(CH₂)₂S—P(O)(OH)₂;
NBD-(CH₂)₂—C(O)—NH—FFFK(Dmt)-C(O)—NH(CH₂)₂S—P(O)(OH)₂;
Naphthyl-(CH₂)—C(O)—NH-fffk(dmt)-C(O)—NH(CH₂)₂S—P(O)(OH)₂;
NBD-(CH₂)₂—C (O)—NH-fffk(dmt)-C(O)—NH(CH₂)₂S—P(O)(OH)₂;
Naphthyl-(CH₂)—C(O)—NH—FFGK(Dmt)-C(O)—NH(CH₂)₂S—P(O)(OH)₂;
NBD-(CH₂)₂—C(O)—NH—FFGK(Dmt)-C(O)—NH(CH₂)₂S—P(O)(OH)₂;
Naphthyl-(CH₂)—C(O)—NH-ffgk(dmt)-C(O)—NH(CH₂)₂S—P(O)(OH)₂;
NBD-(CH₂)₂—C(O)—NH-ffgk(dmt)-C(O)—NH(CH₂)₂S—P(O)(OH)₂;
Naphthyl-(CH₂)—C(O)—NH—FFCY—C(O)—NH(CH₂)₂S—P(O)(OH)₂;
NBD-(CH₂)₂—C(O)—NH—FFCY—C(O)—NH(CH₂)₂S—P(O)(OH)₂;
Naphthyl-(CH₂)—C(O)—NH-ffcy-C(O)—NH(CH₂)₂S—P(O)(OH)₂;
NBD-(CH₂)₂—C(O)—NH-ffcy-C(O)—NH(CH₂)₂S—P(O)(OH)₂;
Naphthyl-(CH₂)—C(O)—NH—FFFCY—C(O)—NH(CH₂)₂S—P(O)(OH)₂;
NBD-(CH₂)₂—C(O)—NH—FFFCY—C(O)—NH(CH₂)₂S—P(O)(OH)₂;
Naphthyl-(CH₂)—C(O)—NH-fffcy-C(O)—NH(CH₂)₂S—P(O)(OH)₂;
NBD-(CH₂)₂—C(O)—NH-fffcy-C(O)—NH(CH₂)₂S—P(O)(OH)₂;
Naphthyl-(CH₂)—C(O)—NH—FFGCY—C(O)—NH(CH₂)₂S—P(O)(OH)₂;
NBD-(CH₂)₂—C(O)—NH—FFGCY—C(O)—NH(CH₂)₂S—P(O)(OH)₂;
Naphthyl-(CH₂)—C(O)—NH-ffgcy-C(O)—NH(CH₂)₂S—P(O)(OH)₂;
NBD-(CH₂)₂—C(O)—NH-ffgcy-C(O)—NH(CH₂)₂S—P(O)(OH)₂;
Naphthyl-(CH₂)—C(O)—NH—FFGC—C(O)—NH(CH₂)₂S—P(O)(OH)₂;
NBD-(CH₂)₂—C(O)—NH—FFGC—C(O)—NH(CH₂)₂S—P(O)(OH)₂;
Naphthyl-(CH₂)—C(O)—NH-ffgc-C(O)—NH(CH₂)₂S—P(O)(OH)₂;
NBD-(CH₂)₂—C(O)—NH-ffgc-C(O)—NH(CH₂)₂S—P(O)(OH)₂;
Naphthyl-(CH₂)—C(O)—NH—FFGCF—C(O)—NH(CH₂)₂S—P(O)(OH)₂;
NBD-(CH₂)₂—C(O)—NH—FFGCF—C(O)—NH(CH₂)₂S—P(O)(OH)₂;
Naphthyl-(CH₂)—C(O)—NH-ffgcf-C(O)—NH(CH₂)₂S—P(O)(OH)₂;
NBD-(CH₂)₂—C(O)—NH-ffgcf-C(O)—NH(CH₂)₂S—P(O)(OH)₂;
Naphthyl-(CH₂)—C(O)—NH—FFC(Dmt)-C(O)—NH(CH₂)₂S—P(O)(OH)₂;
NBD-(CH₂)₂—C(O)—NH—FFC(Dmt)-C(O)—NH(CH₂)₂S—P(O)(OH)₂;
Naphthyl-(CH₂)—C(O)—NH-ffc(dmt)-C(O)—NH(CH₂)₂S—P(O)(OH)₂;
NBD-(CH₂)₂—C(O)—NH-ffc(dmt)-C(O)—NH(CH₂)₂S—P(O)(OH)₂;
Naphthyl-(CH₂)—C(O)—NH—FFFC(Dmt)-C(O)—NH(CH₂)₂S—P(O)(OH)₂;
NBD-(CH₂)₂—C(O)—NH—FFFC(Dmt)-C(O)—NH(CH₂)₂S—P(O)(OH)₂;
Naphthyl-(CH₂)—C(O)—NH-fffc(dmt)-C(O)—NH(CH₂)₂S—P(O)(OH)₂;
NBD-(CH₂)₂—C(O)—NH-fffc(dmt)-C(O)—NH(CH₂)₂S—P(O)(OH)₂;
Naphthyl-(CH₂)—C(O)—NH—FFGC(Dmt)-C(O)—NH(CH₂)₂S—P(O)(OH)₂;
NBD-(CH₂)₂—C(O)—NH—FFGC(Dmt)-C(O)—NH(CH₂)₂S—P(O)(OH)₂;
Naphthyl-(CH₂)—C(O)—NH-ffgc(dmt)-C(O)—NH(CH₂)₂S—P(O)(OH)₂;
NBD-(CH₂)₂—C(O)—NH-ffgc(dmt)-C(O)—NH(CH₂)₂S—P(O)(OH)₂;
Naphthyl-CH₂C(O)—NH—FFK(ε-Z₂)—C(O)—NH(CH₂)₂S—P(O)(OH)₂;
Naphthyl-CH₂C(O)—NH-ffk(ε-Z₂)—C(O)—NH(CH₂)₂S—P(O)(OH)₂;
Naphthyl-CH₂C(O)—NH—FFK(ε-Z₂)Y—C(O)—NH(CH₂)₂S—P(O)(OH)₂;
Naphthyl-CH₂C(O)—NH-ffk(ε-Z₂)y-C(O)—NH(CH₂)₂S—P(O)(OH)₂;
Naphthyl-CH₂C(O)—NH—FFFK(ε-Z₂)Y—C(O)—NH(CH₂)₂S—P(O)(OH)₂;
Naphthyl-CH₂C(O)—NH-fffk(ε-Z₂)y-C(O)—NH(CH₂)₂S—P(O)(OH)₂;
Naphthyl-CH₂C(O)—NH—FFGK(ε-Z₂)Y—C(O)—NH(CH₂)₂S—P(O)(OH)₂;
Naphthyl-CH₂C(O)—NH-ffgk(ε-Z₂)y-C(O)—NH(CH₂)₂S—P(O)(OH)₂;
Naphthyl-CH₂C(O)—NH—FFGK(ε-Z₂)—C(O)—NH(CH₂)₂S—P(O)(OH)₂;
Naphthyl-CH₂C(O)—NH-ffgk(ε-Z₂)—C(O)—NH(CH₂)₂S—P(O)(OH)₂;
Naphthyl-CH₂C(O)—NH—FFGK(ε-Z₂)F—C(O)—NH(CH₂)₂S—P(O)(OH)₂;
Naphthyl-CH₂C(O)—NH-ffgk(ε-Z₂)f-C(O)—NH(CH₂)₂S—P(O)(OH)₂;
Naphthyl-CH₂C(O)—NH—FFK(ε-Z₂)(Dmt)-C(O)—NH(CH₂)₂S—P(O)(OH)₂;
Naphthyl-CH₂C(O)—NH-ffk(ε-Z₂)(dmt)-C(O)—NH(CH₂)₂S—P(O)(OH)₂;
Naphthyl-CH₂C(O)—NH—FFFK(ε-Z₂)(Dmt)-C(O)—NH(CH₂)₂S—P(O)(OH)₂;
Naphthyl-CH₂C(O)—NH-fffk(ε-Z₂)(dmt)-C(O)—NH(CH₂)₂S—P(O)(OH)₂;
Naphthyl-CH₂C(O)—NH—FFGK(ε-Z₂)(Dmt)-C(O)—NH(CH₂)₂S—P(O)(OH)₂;
Naphthyl-CH₂C(O)—NH-ffgk(ε-Z₂)(dmt)-C(O)—NH(CH₂)₂S—P(O)(OH)₂;
NBD-(CH₂)₂C(O)—NH—FFK(ε-Z₂)—C(O)—NH(CH₂)₂S—P(O)(OH)₂;
NBD-(CH₂)₂C(O)—NH-ffk(ε-Z₂)—C(O)—NH(CH₂)₂S—P(O)(OH)₂;
NBD-(CH₂)₂C(O)—NH—FFK(ε-Z₂)Y—C(O)—NH(CH₂)₂S—P(O)(OH)₂;
NBD-(CH₂)₂C(O)—NH-ffk(ε-Z₂)y-C(O)—NH(CH₂)₂S—P(O)(OH)₂;
NBD-(CH₂)₂C(O)—NH—FFFK(ε-Z₂)Y—C(O)—NH(CH₂)₂S—P(O)(OH)₂;
NBD-(CH₂)₂C(O)—NH-fffk(ε-Z₂)y-C(O)—NH(CH₂)₂S—P(O)(OH)₂;
NBD-(CH₂)₂C(O)—NH—FFGK(ε-Z₂)Y—C(O)—NH(CH₂)₂S—P (O)(OH)₂;
NBD-(CH₂)₂C(O)—NH-ffgk(ε-Z₂)y-C(O)—NH(CH₂)₂S—P(O)(OH)₂;
NBD-(CH₂)₂C(O)—NH—FFGK(ε-Z₂)—C(O)—NH(CH₂)₂S—P(O)(OH)₂;
NBD-(CH₂)₂C(O)—NH-ffgk(ε-Z₂)—C(O)—NH(CH₂)₂S—P(O)(OH)₂;
NBD-(CH₂)₂C(O)—NH—FFGK(ε-Z₂)F—C(O)—NH(CH₂)₂S—P(O)(OH)₂;
NBD-(CH₂)₂C(O)—NH-ffgk(ε-Z₂)f-C(O)—NH(CH₂)₂S—P(O)(OH)₂;
NBD-(CH₂)₂C(O)—NH—FFK(ε-Z₂)(Dmt)-C(O)—NH(CH₂)₂S—P (O)(OH)₂;
NBD-(CH₂)₂C(O)—NH-ffk(ε-Z₂)(dmt)—C(O)—NH(CH₂)₂S—P(O)(OH)₂;
NBD-(CH₂)₂C(O)—NH—FFFK(ε-Z₂)(Dmt)-C(O)—NH(CH₂)₂S—P(O)(OH)₂;
NBD-(CH₂)₂C(O)—NH-fffk(ε-Z₂)(dmt)-C(O)—NH(CH₂)₂S—P(O)(OH)₂;
NBD-(CH₂)₂C(O)—NH—FFGK(ε-Z₂)(Dmt)-C(O)—NH(CH₂)₂S—P (O)(OH)₂;
NBD-(CH₂)₂C(O)—NH-ffgk(ε-Z₂)(dmt)-C(O)—NH(CH₂)₂S—P(O)(OH)₂;
Naphthyl-(CH₂)—C(O)—NH—FF—C(O)—NH(CH₂)₃NH(CH₂)₂S—P(O)(OH)₂;
NBD-(CH₂)₂—C(O)—NH—FF—C(O)—NH(CH₂)₃NH(CH₂)₂S—P(O)(OH)₂;
Naphthyl-(CH₂)—C(O)—NH-ff-C(O)—NH(CH₂)₃NH(CH₂)₂S—P(O)(OH)₂;
NBD-(CH₂)₂—C(O)—NH-ff-C(O)—NH(CH₂)₃NH(CH₂)₂S—P(O)(OH)₂;
Naphthyl-(CH₂)—C(O)—NH—FFK—C(O)—NH(CH₂)₃NH(CH₂)₂S—P(O)(OH)₂;
NBD-(CH₂)₂—C(O)—NH—FFK—C(O)—NH(CH₂)₃NH(CH₂)₂S—P(O)(OH)₂;
Naphthyl-(CH₂)—C(O)—NH-ffk-C(O)—NH(CH₂)₃NH(CH₂)₂S—P(O)(OH)₂;
NBD-(CH₂)₂—C(O)—NH-ffk-C(O)—NH(CH₂)₃NH(CH₂)₂S—P(O)(OH)₂;
Naphthyl-(CH₂)—C(O)—NH—FFKY—C(O)—NH(CH₂)₃NH(CH₂)₂S—P(O)(OH)₂;
NBD-(CH₂)₂—C(O)—NH—FFKY—C(O)—NH(CH₂)₃NH(CH₂)₂S—P(O)(OH)₂;
Naphthyl-(CH₂)—C(O)—NH-ffky-C(O)—NH(CH₂)₃NH(CH₂)₂S—P(O)(OH)₂;
NBD-(CH₂)₂—C(O)—NH-ffky-C(O)—NH(CH₂)₃NH(CH₂)₂S—P(O)(OH)₂;
Naphthyl-(CH₂)—C(O)—NH—FFFKY—C(O)—NH(CH₂)₃NH(CH₂)₂S—P(O)(OH)₂;
NBD-(CH₂)₂—C(O)—NH—FFFKY—C(O)—NH(CH₂)₃NH(CH₂)₂S—P(O)(OH)₂;
Naphthyl-(CH₂)—C(O)—NH-fffky-C(O)—NH(CH₂)₃NH(CH₂)₂S—P(O)(OH)₂;
NBD-(CH₂)₂—C(O)—NH-fffky-C(O)—NH(CH₂)₃NH(CH₂)₂S—P(O)(OH)₂;
Naphthyl-(CH₂)—C(O)—NH—FFGKY—C(O)—NH(CH₂)₃NH(CH₂)₂S—P(O)(OH)₂;
NBD-(CH₂)₂—C(O)—NH—FFGKY—C(O)—NH(CH₂)₃NH(CH₂)₂S—P(O)(OH)₂;
Naphthyl-(CH₂)—C(O)—NH-ffgky-C(O)—NH(CH₂)₃NH(CH₂)₂S—P(O)(OH)₂;
NBD-(CH₂)₂—C(O)—NH-ffgky-C(O)—NH(CH₂)₃NH(CH₂)₂S—P(O)(OH)₂;
Naphthyl-(CH₂)—C(O)—NH—FFGK—C(O)—NH(CH₂)₃NH(CH₂)₂S—P(O)(OH)₂;
NBD-(CH₂)₂—C(O)—NH—FFGK—C(O)—NH(CH₂)₃NH(CH₂)₂S—P(O)(OH)₂;
Naphthyl-(CH₂)—C(O)—NH-ffgk-C(O)—NH(CH₂)₃NH(CH₂)₂S—P(O)(OH)₂;
NBD-(CH₂)₂—C(O)—NH-ffgk-C(O)—NH(CH₂)₃NH(CH₂)₂S—P(O)(OH)₂;
Naphthyl-(CH₂)—C(O)—NH—FFGKF—C(O)—NH(CH₂)₃NH(CH₂)₂S—P(O)(OH)₂;
NBD-(CH₂)₂—C(O)—NH—FFGKF—C(O)—NH(CH₂)₃NH(CH₂)₂S—P(O)(OH)₂;
Naphthyl-(CH₂)—C(O)—NH-ffgkf-C(O)—NH(CH₂)₃NH(CH₂)₂S—P(O)(OH)₂;
NBD-(CH₂)₂—C(O)—NH-ffgkf-C(O)—NH(CH₂)₃NH(CH₂)₂S—P(O)(OH)₂;
Naphthyl-(CH₂)—C(O)—NH—FFK(Dmt)-C(O)—NH(CH₂)₃NH(CH₂)₂S—P(O)(OH)₂;
NBD-(CH₂)₂—C(O)—NH—FFK(Dmt)-C(O)—NH(CH₂)₃NH(CH₂)₂S—P(O)(OH)₂;
Naphthyl-(CH₂)—C(O)—NH-ffk(dmt)-C(O)—NH(CH₂)₃NH(CH₂)₂S—P(O)(OH)₂;
NBD-(CH₂)₂—C(O)—NH-ffk(dmt)-C(O)—NH(CH₂)₃NH(CH₂)₂S—P (O)(OH)₂;
Naphthyl-(CH₂)—C(O)—NH—FFFK(Dmt)-C(O)—NH(CH₂)₃NH(CH₂)₂S—P(O)(OH)₂;
NBD-(CH₂)₂—C(O)—NH—FFFK(Dmt)-C(O)—NH(CH₂)₃NH(CH₂)₂S—P(O)(OH)₂;
Naphthyl-(CH₂)—C(O)—NH-fffk(dmt)-C(O)—NH(CH₂)₃NH(CH₂)₂S—P(O)(OH)₂;
NBD-(CH₂)₂—C(O)—NH-fffk(dmt)-C(O)—NH(CH₂)₃NH(CH₂)₂S—P (O)(OH)₂;
Naphthyl-(CH₂)—C(O)—NH—FFGK(Dmt)-C(O)—NH(CH₂)₃NH(CH₂)₂S—P(O)(OH)₂;
NBD-(CH₂)₂—C(O)—NH—FFGK(Dmt)-C(O)—NH(CH₂)₃NH(CH₂)₂S—P (O)(OH)₂;
Naphthyl-(CH₂)—C(O)—NH-ffgk(dmt)-C(O)—NH(CH₂)₃NH(CH₂)₂S—P(O)(OH)₂;
NBD-(CH₂)₂—C(O)—NH-ffgk(dmt)-C(O)—NH(CH₂)₃NH(CH₂)₂S—P (O)(OH)₂;
Naphthyl-(CH₂)—C(O)—NH—FFCY—C(O)—NH(CH₂)₃NH(CH₂)₂S—P(O)(OH)₂;
NBD-(CH₂)₂—C(O)—NH—FFCY—C(O)—NH(CH₂)₃NH(CH₂)₂S—P (O)(OH)₂;
Naphthyl-(CH₂)—C(O)—NH-ffcy-C(O)—NH(CH₂)₃NH(CH₂)₂S—P(O)(OH)₂;
NBD-(CH₂)₂—C(O)—NH-ffcy-C(O)—NH(CH₂)₃NH(CH₂)₂S—P(O)(OH)₂;
Naphthyl-(CH₂)—C(O)—NH—FFFCY—C(O)—NH(CH₂)₃NH(CH₂)₂S—P (O)(OH)₂;
NBD-(CH₂)₂—C(O)—NH—FFFCY—C(O)—NH(CH₂)₃NH(CH₂)₂S—P(O)(OH)₂;
Naphthyl-(CH₂)—C(O)—NH-fffcy-C(O)—NH(CH₂)₃NH(CH₂)₂S—P(O)(OH)₂;
NBD-(CH₂)₂—C(O)—NH-fffcy-C(O)—NH(CH₂)₃NH(CH₂)₂S—P(O)(OH)₂;
Naphthyl-(CH₂)—C(O)—NH—FFGCY—C(O)—NH(CH₂)₃NH(CH₂)₂S—P(O)(OH)₂;
NBD-(CH₂)₂—C(O)—NH—FFGCY—C(O)—NH(CH₂)₃NH(CH₂)₂S—P(O)(OH)₂;
Naphthyl-(CH₂)—C(O)—NH-ffgcy-C(O)—NH(CH₂)₃NH(CH₂)₂S—P(O)(OH)₂;
NBD-(CH₂)₂—C(O)—NH-ffgcy-C(O)—NH(CH₂)₃NH(CH₂)₂S—P(O)(OH)₂;
Naphthyl-(CH₂)—C(O)—NH—FFGC—C(O)—NH(CH₂)₃NH(CH₂)₂S—P(O)(OH)₂;
NBD-(CH₂)₂—C(O)—NH—FFGC—C(O)—NH(CH₂)₃NH(CH₂)₂S—P (O)(OH)₂;
Naphthyl-(CH₂)—C(O)—NH-ffgc-C(O)—NH(CH₂)₃NH(CH₂)₂S—P(O)(OH)₂;
NBD-(CH₂)₂—C(O)—NH-ffgc-C(O)—NH(CH₂)₃NH(CH₂)₂S—P(O)(OH)₂;
Naphthyl-(CH₂)—C(O)—NH—FFGCF—C(O)—NH(CH₂)₃NH(CH₂)₂S—P (O)(OH)₂;
NBD-(CH₂)₂—C(O)—NH—FFGCF—C(O)—NH(CH₂)₃NH(CH₂)₂S—P(O)(OH)₂;
Naphthyl-(CH₂)—C(O)—NH-ffgcf-C(O)—NH(CH₂)₃NH(CH₂)₂S—P(O)(OH)₂;
NBD-(CH₂)₂—C(O)—NH-ffgcf-C(O)—NH(CH₂)₃NH(CH₂)₂S—P(O)(OH)₂;
Naphthyl-(CH₂)—C(O)—NH—FFC(Dmt)-C(O)—NH(CH₂)₃NH(CH₂)₂S—P(O)(OH)₂;
NBD-(CH₂)₂—C(O)—NH—FFC(Dmt)-C(O)—NH(CH₂)₃NH(CH₂)₂S—P(O)(OH)₂;
Naphthyl-(CH₂)—C(O)—NH-ffc(dmt)-C(O)—NH(CH₂)₃NH(CH₂)₂S—P(O)(OH)₂;
NBD-(CH₂)₂—C(O)—NH-ffc(dmt)-C(O)—NH(CH₂)₃NH(CH₂)₂S—P(O)(OH)₂;
Naphthyl-(CH₂)—C(O)—NH—FFFC(Dmt)-C(O)—NH(CH₂)₃NH(CH₂)₂S—P(O)(OH)₂;
NBD-(CH₂)₂—C(O)—NH—FFFC(Dmt)-C(O)—NH(CH₂)₃NH(CH₂)₂S—P(O)(OH)₂;
Naphthyl-(CH₂)—C(O)—NH-fffc(dmt)-C(O)—NH(CH₂)₃NH(CH₂)₂S—P(O)(OH)₂;
NBD-(CH₂)₂—C(O)—NH-fffc(dmt)-C(O)—NH(CH₂)₃NH(CH₂)₂S—P(O)(OH)₂;
Naphthyl-(CH₂)—C(O)—NH—FFGC(Dmt)-C(O)—NH(CH₂)₃NH(CH₂)₂S—P(O)(OH)₂;
NBD-(CH₂)₂—C(O)—NH—FFGC(Dmt)-C(O)—NH(CH₂)₃NH(CH₂)₂S—P(O)(OH)₂;
Naphthyl-(CH₂)—C(O)—NH-ffgc(dmt)-C(O)—NH(CH₂)₃NH(CH₂)₂S—P(O)(OH)₂;
NBD-(CH₂)₂—C(O)—NH-ffgc(dmt)-C(O)—NH(CH₂)₃NH(CH₂)₂S—P(O)(OH)₂;
Naphthyl-CH₂C(O)—NH—FFK(ε-Z₂)—C(O)—NH(CH₂)₃NH(CH₂)₂S—P(O)(OH)2;
Naphthyl-CH₂C(O)—NH-ffk(ε-Z₂)—C(O)—NH(CH₂)₃NH(CH₂)₂S—P(O)(OH)₂;
Naphthyl-CH₂C(O)—NH—FFK(ε-Z₂)Y—C(O)—NH(CH₂)₃NH(CH₂)₂S—P(O)(OH)₂;
Naphthyl-CH₂C(O)—NH-ffk(ε-Z₂)y-C(O)—NH(CH₂)₃NH(CH₂)₂S—P(O)(OH)₂;
Naphthyl-CH₂C(O)—NH—FFFK(ε-Z₂)Y—C(O)—NH(CH₂)₃NH(CH₂)₂S—P(O)(OH)₂;
Naphthyl-CH₂C(O)—NH—fffk(ε-Z₂)y-C(O)—NH(CH₂)₃NH(CH₂)₂S—P(O)(OH)₂;
Naphthyl-CH₂C(O)—NH-FFGK(ε-Z₂)Y—C(O)—NH(CH₂)₃NH(CH₂)₂S—P(O)(OH)₂;
Naphthyl-CH₂C(O)—NH-ffgk(ε-Z₂)y-C(O)—NH(CH₂)₃NH(CH₂)₂S—P(O)(OH)₂;
Naphthyl-CH₂C(O)—NH—FFGK(ε-Z₂)—C(O)—NH(CH₂)₃NH(CH₂)₂S—P(O)(OH)₂;
Naphthyl-CH₂C(O)—NH-ffgk(ε-Z₂)—C(O)—NH(CH₂)₃NH(CH₂)₂S—P(O)(OH)₂;
Naphthyl-CH₂C(O)—NH—FFGK(ε-Z₂)F—C(O)—NH(CH₂)₃NH(CH₂)₂S—P(O)(OH)₂;
Naphthyl-CH₂C(O)—NH-ffgk(ε-Z₂)f-C(O)—NH(CH₂)₃NH(CH₂)₂S—P(O)(OH)₂;
Naphthyl-CH₂C(O)—NH—FFK(ε-Z₂)(Dmt)-C(O)—NH(CH₂)₃NH(CH₂)₂S—P(O)(OH)₂;
Naphthyl-CH₂C(O)—NH-ffk(ε-Z₂)(dmt)-C(O)—NH(CH₂)₃NH(CH₂)₂S—P(O)(OH)₂;
Naphthyl-CH₂C(O)—NH—FFFK(ε-Z₂)(Dmt)-C(O)—NH(CH₂)₃NH(CH₂)₂S—P(O)(OH)₂;
Naphthyl-CH₂C(O)—NH-fffk(ε-Z₂)(dmt)-C(O)—NH(CH₂)₃NH(CH₂)₂S—P(O)(OH)₂;
Naphthyl-CH₂C(O)—NH—FFGK(ε-Z₂)(Dmt)-C(O)—NH(CH₂)₃NH(CH₂)₂S—P (O)(OH)₂;
Naphthyl-CH₂C(O)—NH-ffgk(ε-Z₂)(dmt)-C(O)—NH(CH₂)₃NH(CH₂)₂S—P(O)(OH)₂;
NBD-(CH₂)₂—C(O)—NH—FFK(ε-Z₂)—C(O)—NH(CH₂)₃NH(CH₂)₂S—P(O)(OH)₂;
NBD-(CH₂)₂—C(O)—NH-ffk(249 -Z₂)—C(O)—NH(CH₂)₃NH(CH₂)₂S—P(O)(OH)₂;
NBD-(CH₂)₂—C(O)—NH—FFK(ε-Z₂)Y—C(O)—NH(CH₂)₃NH(CH₂)₂S—P(O)(OH)₂;
NBD-(CH₂)₂—C(O)—NH-ffk(ε-Z₂)y-C(O)—NH(CH₂)₃NH(CH₂)₂S—P(O)(OH)₂;
NBD-(CH₂)₂—C(O)—NH—FFFK(ε-Z₂)Y—C(O)—NH(CH₂)₃NH(CH₂)₂S—P(O)(OH)₂;
NBD-(CH₂)₂—C(O)—NH-fffk(ε-Z₂)y-C(O)—NH(CH₂)₃NH(CH₂)₂S—P(O)(OH)₂;
NBD-(CH₂)₂—C(O)—NH—FFGK(ε-Z₂)Y—C(O)—NH(CH₂)₃NH(CH₂)₂S—P(O)(OH)₂;
NBD-(CH₂)₂—C(O)—NH-ffgk(ε-Z₂)y-CO)—NH(CH₂)₃NH(CH₂)₂S—P(O)(OH)₂;
NBD-(CH₂)₂—C(O)—NH—FFGK(ε-Z₂)—C(O)—NH(CH₂)₃NH(CH₂)₂S—P(O)(OH)₂;
NBD (CH₂)₂—C(O)—NH-ffgk(ε-Z₂)—C(O)—NH(CH₂)₃NH(CH₂)₂S—P(O)(OH)₂;
NBD-(CH₂)₂—C(O)—NH—FFGK(ε-Z₂)F—C(O)—NH(CH₂)₃NH(CH₂)₂S—P(O)(OH)₂;
NBD-(CH₂)₂—C(O)—NH-ffgk(ε-Z₂)f-C(O)—NH(CH₂)₃NH(CH₂)₂S—P(O)(OH)₂;
NBD-(CH₂)₂—C(O)—NH—FFK(ε-Z₂)(Dmt)-C(O)—NH(CH₂)₃NH(CH₂)₂S—P(O)(OH)₂;
NBD-(CH₂)₂—C(O)—NH-ffk(ε-Z₂)(dmt)—C(O)—NH(CH₂)₃NH(CH₂)₂S—P(O)(OH)₂;
NBD-(CH₂)₂—C(O)—NH—FFFK(ε-Z₂)(Dmt)-C(O)—NH(CH₂)₃NH(CH₂)₂S—P(O)(OH)₂;
NBD (CH₂)₂—C(O)—NH-fffk(ε-Z₂)(dmt)-C(O)—NH(CH₂)₃NH(CH₂)₂S—P(O)(OH)₂;
NBD-(CH₂)₂—C(O)—NH—FFGK(ε-Z₂)(Dmt)-C(O)—NH(CH₂)₃NH(CH₂)₂S—P(O)(OH)₂;
NBD-(CH₂)₂—C(O)—NH-ffgk(ε-Z₂)(dmt)-C(O)—NH(CH₂)₃NH(CH₂)₂S—P(O)(OH)₂;
Naphthyl-(CH₂)—C(O)—NH—FF—C(O)—NH(CH₂)₂S—C(O)CH₃;
NBD-(CH₂)₂—C(O)—NH—FF—C(O)—NH(CH₂)₂S—C(O)CH₃;
Naphthyl-(CH₂)—C(O)—NH-ff-C(O)—NH(CH₂)₂S—C(O)CH₃;
NBD-(CH₂)₂—C(O)—NH-ff-C(O)—NH(CH₂)₂S—C(O)CH₃;
Naphthyl-(CH₂)—C(O)—NH—FFK—C(O)—NH(CH₂)₂S—C(O)CH₃;
NBD-(CH₂)₂—C(O)—NH—FFK—C(O)—NH(CH₂)₂S—C(O)CH₃;
Naphthyl-(CH₂)—C(O)—NH-ffk-C(O)—NH(CH₂)₂S—C(O)CH₃;
NBD-(CH₂)₂—C(O)—NH-ffk-C(O)—NH(CH₂)₂S—C(O)CH₃;
Naphthyl-(CH₂)—C(O)—NH—FFKY—C(O)—NH(CH₂)₂S—C(O)CH₃;
NBD-(CH₂)₂—C(O)—NH—FFKY—C(O)—NH(CH₂)₂S—C(O)CH₃;
Naphthyl-(CH₂)—C(O)—NH-ffky-C(O)—NH(CH₂)₂S—C(O)CH₃;
NBD-(CH₂)₂—C(O)—NH-ffky-C(O)—NH(CH₂)₂S—C(O)CH₃;
Naphthyl-(CH₂)—C(O)—NH—FFFKY—C(O)—NH(CH₂)₂S—C(O)CH₃;
NBD-(CH₂)₂—C(O)—NH—FFFKY—C(O)—NH(CH₂)₂S—C(O)CH₃;
Naphthyl-(CH₂)—C(O)—NH-fffky-C(O)—NH(CH₂)₂S—C(O)CH₃;
NBD-(CH₂)₂—C(O)—NH-fffky-C(O)—NH(CH₂)₂S—C(O)CH₃;
Naphthyl-(CH₂)—C(O)—NH—FFGKY—C(O)—NH(CH₂)₂S—C(O)CH₃;
NBD-(CH₂)₂—C(O)—NH—FFGKY—C(O)—NH(CH₂)₂S—C(O)CH₃;
Naphthyl-(CH₂)—C(O)—NH-ffgky-C(O)—NH(CH₂)₂S—C(O)CH₃;
NBD-(CH₂)₂—C(O)—NH-ffgky-C(O)—NH(CH₂)₂S—C(O)CH₃;
Naphthyl-(CH₂)—C(O)—NH—FFGK—C(O)—NH(CH₂)₂S—C(O)CH₃;
NBD-(CH₂)₂—C(O)—NH—FFGK—C(O)—NH(CH₂)₂S—C(O)CH₃;
Naphthyl-(CH₂)—C(O)—NH-ffgk-C(O)—NH(CH₂)₂S—C(O)CH₃;
NBD-(CH₂)₂—C(O)—NH-ffgk-C(O)—NH(CH₂)₂S—C(O)CH₃;
Naphthyl-(CH₂)—C(O)—NH—FFGKF—C(O)—NH(CH₂)₂S—C(O)CH₃;
NBD-(CH₂)₂—C(O)—NH—FFGKF—C(O)—NH(CH₂)₂S—C(O)CH₃;
Naphthyl-(CH₂)—C(O)—NH-ffgkf-C(O)—NH(CH₂)₂S—C(O)CH₃;
NBD-(CH₂)₂—C(O)—NH-ffgkf-C(O)—NH(CH₂)₂S—C(O)CH₃;
Naphthyl-(CH₂)—C(O)—NH—FFK(Dmt)-C(O)—NH(CH₂)₂S—C(O)CH₃;
NBD-(CH₂)₂—C(O)—NH—FFK(Dmt)-C(O)—NH(CH₂)₂S—C(O)CH₃;
Naphthyl-(CH₂)—C(O)—NH-ffk(dmt)-C(O)—NH(CH₂)₂S—C(O)CH₃;
NBD-(CH₂)₂—C(O)—NH-ffk(dmt)-C(O)—NH(CH₂)₂S—C(O)CH₃;
Naphthyl-(CH₂)—C(O)—NH—FFFK(Dmt)-C(O)—NH(CH₂)₂S—C(O)CH₃;
NBD-(CH₂)₂—C(O)—NH—FFFK(Dmt)-C(O)—NH(CH₂)₂S—C(O)CH₃;
Naphthyl-(CH₂)—C(O)—NH-fffk(dmt)-C(O)—NH(CH₂)₂S—C(O)CH₃;
NBD-(CH₂)₂—C(O)—NH-fffk(dmt)-C(O)—NH(CH₂)₂S—C(O)CH₃;
Naphthyl-(CH₂)—C(O)—NH—FFGK(Dmt)-C(O)—NH(CH₂)₂S—C(O)CH₃;
NBD-(CH₂)₂—C(O)—NH—FFGK(Dmt)-C(O)—NH(CH₂)₂S—C(O)CH₃;
Naphthyl-(CH₂)—C(O)—NH-ffgk(dmt)-C(O)—NH(CH₂)₂S—C(O)CH₃;
NBD-(CH₂)₂—C(O)—NH-ffgk(dmt)-C(O)—NH(CH₂)₂S—C(O)CH₃;
Naphthyl-(CH₂)—C(O)—NH—FFCY—C(O)—NH(CH₂)₂S—C(O)CH₃;
NBD-(CH₂)₂—C(O)—NH—FFCY—C(O)—NH(CH₂)₂S—C(O)CH₃;
Naphthyl-(CH₂)—C(O)—NH-ffcy-C(O)—NH(CH₂)₂S—C(O)CH₃;
NBD-(CH₂)₂—C(O)—NH-ffcy-C(O)—NH(CH₂)₂S—C(O)CH₃;
Naphthyl-(CH₂)—C(O)—NH—FFFCY—C(O)—NH(CH₂)₂S—C(O)CH₃;
NBD-(CH₂)₂—C(O)—NH—FFFCY—C(O)—NH(CH₂)₂S—C(O)CH₃;
Naphthyl-(CH₂)—C(O)—NH-fffcy-C(O)—NH(CH₂)₂S—C(O)CH₃;
NBD-(CH₂)₂—C(O)—NH-fffcy-C(O)—NH(CH₂)₂S—C(O)CH₃;
Naphthyl-(CH₂)—C(O)—NH—FFGCY—C(O)—NH(CH₂)₂S—C(O)CH₃;
NBD-(CH₂)₂—C(O)—NH—FFGCY—C(O)—NH(CH₂)₂S—C(O)CH₃;
Naphthyl-(CH₂)—C(O)—NH-ffgcy-C(O)—NH(CH₂)₂S—C(O)CH₃;
NBD-(CH₂)₂—C(O)—NH-ffgcy-C(O)—NH(CH₂)₂S—C(O)CH₃;
Naphthyl-(CH₂)—C(O)—NH—FFGC—C(O)—NH(CH₂)₂S—C(O)CH₃;
NBD-(CH₂)₂—C(O)—NH—FFGC—C(O)—NH(CH₂)₂S—C(O)CH₃;
Naphthyl-(CH₂)—C(O)—NH-ffgc-C(O)—NH(CH₂)₂S—C(O)CH₃;
NBD-(CH₂)₂—C(O)—NH-ffgc-C(O)—NH(CH₂)₂S—C(O)CH₃;
Naphthyl-(CH₂)—C(O)—NH—FFGCF—C(O)—NH(CH₂)₂S—C(O)CH₃;
NBD-(CH₂)₂—C(O)—NH—FFGCF—C(O)—NH(CH₂)₂S—C(O)CH₃;
Naphthyl-(CH₂)—C(O)—NH-ffgcf-C(O)—NH(CH₂)₂S—C(O)CH₃;
NBD-(CH₂)₂—C(O)—NH-ffgcf-C(O)—NH(CH₂)₂S—C(O)CH₃;
Naphthyl-(CH₂)—C(O)—NH—FFC(Dmt)-C(O)—NH(CH₂)₂S—C(O)CH₃;
NBD-(CH₂)₂—C(O)—NH—FFC(Dmt)-C(O)—NH(CH₂)₂S—C(O)CH₃;
Naphthyl-(CH₂)—C(O)—NH-ffc(dmt)-C(O)—NH(CH₂)₂S—C(O)CH₃;
NBD-(CH₂)₂—C(O)—NH-ffc(dmt)-C(O)—NH(CH₂)₂S—C(O)CH₃;
Naphthyl-(CH₂)—C(O)—NH-FFFC(Dmt)-C(O)—NH(CH₂)₂S—C(O)CH₃;
NBD-(CH₂)₂—C(O)—NH—FFFC(Dmt)-C(O)—NH(CH₂)₂S—C(O)CH₃;
Naphthyl-(CH₂)—C(O)—NH-fffc(dmt)-C(O)—NH(CH₂)₂S—C(O)CH₃;
NBD-(CH₂)₂—C(O)—NH-fffc(dmt)-C(O)—NH(CH₂)₂S—C(O)CH₃;
Naphthyl-(CH₂)—C(O)—NH—FFGC(Dmt)-C(O)—NH(CH₂)₂S—C(O)CH₃;
NBD-(CH₂)₂—C(O)—NH—FFGC(Dmt)-C(O)—NH(CH₂)₂S—C(O)CH₃;
Naphthyl-(CH₂)—C(O)—NH-ffgc(dmt)-C(O)—NH(CH₂)₂S—C(O)CH₃;
NBD-(CH₂)₂—C(O)—NH-ffgc(dmt)-C(O)—NH(CH₂)₂S—C(O)CH₃;
Naphthyl-CH₂C(O)—NH—FFK(ε-Z₂)—C(O)—NH(CH₂)₂S—C(O)CH₃;
Naphthyl-CH₂C(O)—NH—ffk(ε-Z₂)—C(O)—NH(CH₂)₂S—C(O)CH₃;
Naphthyl-CH₂C(O)—NH—FFK(ε-Z₂)Y—C(O)—NH(CH₂)₂S—C(O)CH₃;
Naphthyl-CH₂C(O)—NH—ffk(ε-Z₂)y-C(O)—NH(CH₂)₂S—C(O)CH₃;
Naphthyl-CH₂C(O)—NH—FFFK(ε-Z₂)Y—C(O)—NH(CH₂)₂S—C(O)CH₃;
Naphthyl-CH₂C(O)—NH—fffk(ε-Z₂)y-C(O)—NH(CH₂)₂S—C(O)CH₃;
Naphthyl-CH₂C(O)—NH—FFGK(ε-Z₂)Y—C(O)—NH(CH₂)₂S—C(O)CH₃;
Naphthyl-CH₂C(O)—NH—ffgk(ε-Z₂)y-C(O)—NH(CH₂)₂S—C(O)CH₃;
Naphthyl-CH₂C(O)—NH—FFGK(ε-Z₂)—C(O)—NH(CH₂)₂S—C(O)CH₃;
Naphthyl-CH₂C(O)—NH—ffgk(ε-Z₂)—C(O)—NH(CH₂)₂S—C(O)CH₃;
Naphthyl-CH₂C(O)—NH—FFGK(ε-Z₂)F—C(O)—NH(CH₂)₂S—C(O)CH₃;
Naphthyl-CH₂C(O)—NH—ffgk(ε-Z₂)f-C(O)—NH(CH₂)₂S—C(O)CH₃;
Naphthyl-CH₂C(O)—NH—FFK(ε-Z₂)(Dmt)-C(O)—NH(CH₂)₂S—C(O)CH₃;
Naphthyl-CH₂C(O)—NH—ffk(ε-Z₂)(dmt)-C(O)—NH(CH₂)₂S—C(O)CH₃;
Naphthyl-CH₂C(O)—NH—FFFK(ε-Z₂)(Dmt)-C(O)—NH(CH₂)₂S—C(O)CH₃;
Naphthyl-CH₂C(O)—NH—FFGK(ε-Z₂)(Dmt)-C(O)—NH(CH₂)₂S—C(O)CH₃;
Naphthyl-CH₂C(O)—NH-ffgk(ε-Z₂)(dmt)-C(O)—NH(CH₂)₂S—C(O)CH₃;
NBD-(CH₂)₂C(O)—NH—FFK(ε-Z₂)—C(O)—NH(CH₂)₂S—C(O)CH₃;
NBD-(CH₂)₂C(O)—NH-ffk(ε-Z₂)—C(O)—NH(CH₂)₂S—C(O)CH₃;
NBD-(CH₂)₂C(O)—NH—FFK(ε-Z₂)Y—C(O)—NH(CH₂)₂S—C(O)CH₃;
NBD-(CH₂)₂C(O)—NH-ffk(ε-Z₂)y-C(O)—NH(CH₂)₂S—C(O)CH₃;
NBD-(CH₂)₂C(O)—NH—FFFK(ε-Z₂)Y—C(O)—NH(CH₂)₂S—C(O)CH₃;
NBD-(CH₂)₂C(O)—NH-fffk(ε-Z₂)y-C(O)—NH(CH₂)₂S—C(O)CH₃;
NBD-(CH₂)₂C(O)—NH—FFGK(ε-Z₂)Y—C(O)—NH(CH₂)₂S—C(O)CH₃;
NBD-(CH₂)₂C(O)—NH-ffgk(ε-Z₂)y-C(O)—NH(CH₂)₂S—C(O)CH₃;
NBD-(CH₂)₂C(O)—NH—FFGK(ε-Z₂)—C(O)—NH(CH₂)₂S—C(O)CH₃;
NBD-(CH₂)₂C(O)—NH-ffgk(ε-Z₂)—C(O)—NH(CH₂)₂S—C(O)CH₃;
NBD-(CH₂)₂C(O)—NH—FFGK(ε-Z₂)F—C(O)—NH(CH₂)₂S—C(O)CH₃;
NBD-(CH₂)₂C(O)—NH-ffgk(ε-Z₂)f-C(O)—NH(CH₂)₂S—C(O)CH₃;
NBD-(CH₂)₂C(O)—NH—FFK(ε-Z₂)(Dmt)-C(O)—NH(CH₂)₂S—C(O)CH₃;
NBD-(CH₂)₂C(O)—NH-ffk(ε-Z₂)(dmt)-C(O)—NH(CH₂)₂S—C(O)CH₃;
NBD-(CH₂)₂C(O)—NH—FFFK(ε-Z₂)(Dmt)-C(O)—NH(CH₂)₂S—C(O)CH₃;
NBD-(CH₂)₂C(O)—NH-fffk(ε-Z₂)(dmt)-C(O)—NH(CH₂)₂S—C(O)CH₃;
NBD-(CH₂)₂C(O)—NH—FFGK(ε-Z₂)(Dmt)-C(O)—NH(CH₂)₂S—C(O)CH₃;
NBD-(CH₂)₂C(O)—NH-ffgk(ε-Z₂)(dmt)-C(O)—NH(CH₂)₂S—C(O)CH₃;
   wherein Dmt is 2,6-dimethyl-L-tyrosine; dmt is 2,6-dimethyl-D-tyrosine; NBD is 4-nitro-2,1,3-benzoxadiazolyl; and Z₂ is defined herein. NBD-(CH₂)₂—C(O) denotes NBD-β-Ala.

According to one approach, the peptides of the present invention can be synthesized by standard peptide synthesis operations. These include both FMOC (9-fluorenylmethyloxy-carbonyl) and tBoc (tert-butyloxy-carbonyl) synthesis protocols that can be carried out on automated solid phase peptide synthesis instruments including, without limitation, the Applied Biosystems 431 A, 433 A synthesizers and Peptide Technologies Symphony or large scale Sonata or CEM Liberty automated solid phase peptide synthesizers. The use of alternative peptide synthesis instruments is also contemplated. Peptides prepared using solid phase synthesis are recovered in a substantially pure form.

Coupling of the thiophosphate moiety or thioester moiety can be carried out by reacting the peptide (e.g., resulting from solid peptide synthesis) with a thiophosphate containing moiety or thioester containing moiety under suitable reaction conditions. By way of example, the peptide can be dissolved in DMF with HBTU, followed by the addition of amifostine (or salt thereof), cysteamine S-phosphate (or salt thereof), or an S-(2-aminoalkyl) alkylthioate. The resulting peptide product can be purified using HPLC.

In certain embodiments, the peptide chain further includes, as Z₁ and/or Z₂, a therapeutic agent covalently bonded to the N-terminal residue of peptide chain or a sidechain of an amino acid residue within the peptide chains, as noted above. Covalent attachment may be carried out directly to the N-terminus or the sidechain of an reactive sidechain. The therapeutic agent, Z₁ and/or Z₂, can be any type of therapeutic agent that is capable of being so-modified. Exemplary therapeutic agents include, without limitation, antioxidants, coenzymes, vitamins, metabolites, analgesics, anti-inflammatory agents, antihelminthics, anti-arrhythmic agents, anti-bacterial agents, anti-viral agents, anti-coagulants, anti-depressants, anti-diabetics, anti-epileptics, anti-fungal agents, anti-gout agents, anti-hypertensive agents, anti-thrombogenic agents, anti-claudication agents, anti-atherosclerotic drugs, vascular agents, anti-malarials, anti-migraine agents, anti-muscarinic agents, anti-neoplastic agents, erectile dysfunction improvement agents, immunosuppressants, anti-protozoal agents, anti-thyroid agents, anxiolytic agents, sedatives, hypnotics, neuroleptics, β-blockers, cardiac inotropic agents, corticosteroids, diuretics, anti-parkinsonian agents, gastro-intestinal agents, histamine receptor antagonists, keratolyptics, lipid regulating agents, anti-anginal agents, Cox-2 inhibitors, leukotriene inhibitors, macrolides, muscle relaxants, nutritional agents, opioid analgesics, protease inhibitors, sex hormones, stimulants, anti-osteoporosis agents, anti-obesity agents, cognition enhancers, anti-urinary incontinence agents, anti-benign prostate hypertrophy agents, essential fatty acids, non-essential fatty acids, cytokines, growth factors, antibodies, radioprotective agents, and cardioprotective agents.

Any of a variety of fluorophore molecules or fluorescent nanoparticles can be used as the Z₁ or Z₂ moiety. Exemplary fluorophores include, without limitation, 4-nitro-2,1,3-benzoxadiazolyl, 5-(dimethylamino)naphthalene-1-sulfonyl, 4-(N,N-dimethylaminosulfonyl)-2,1,3-benzoxadiazolyl, and 9-acridinyl. The accompanying examples demonstrate use of 4-nitro-2,1,3-benzoxadiazolyl. 5-(dimethylamino)naphthalene-1-sulfonyl chloride is a reagent that reacts with primary amino groups in both aliphatic and aromatic amines to produce stable blue- or blue-green-fluorescent sulfonamide adducts. 4-(N,N-Dimethyl aminosulfonyl)-7-isothiocyanato-2,1,3-benzoxadiazole can be used to react with primary amines to afford a stable and strong fluorophore (Matsunaga et al., Anal. Chem. 67(23):4276-82 (1995), which is hereby incorporated by reference in its entirety). 9-acridinyl carboxylic acid can be used to label primary amines in peptides (Carlson et al., Org. Lett. 2(10):1465-1468 (2000), which is hereby incorporated by reference in its entirety).

Several classes of nanoparticles are known in the prior art and the skilled person can select the appropriate type of nanoparticle according to the specific therapeutic or diagnostic requirements.

Examples of nanoparticles to be coupled with the peptide are quantum dots, Noble metal clusters, superparamagnetic iron oxide nanoparticles (IONPs), block-copolymer micelles, nanocells, dendrimers, nanotubes, polymersomes, XPclad™ nanoparticles, and nanoparticles consisting of amorphous silica surrounded by a crystalline luminescent calcium phosphate layer (e.g., ORMOBEAD™).

The ORMOBEAD particles can be suitably modified on the surface with polyethylene imine or TRIAMO yielding amine groups or 6-amino hexanoic acid (AHA) or with adipic acid yielding carboxyl groups. These groups can be used for the coupling to the peptides of the invention. The ORMOBEAD technology is disclosed by Dembski et al. (News Analytik, p. 1-3 (2013), which is hereby incorporated by reference in its entirety).

In one embodiment of the invention SiO₂/Zn₂SiO₄:Mn²⁺ and SiO₂/Ca₁₀ (PO₄)₆OH:Eu³⁺ core-shell nanoparticles with diameters below 100 nm are used as nanoparticles for coupling with the peptides of the invention. These particles are disclosed by Dembski et al., (Optical Materials, 1106-1110 (2011); and GIT-Labor-Fachzeitschrift, p. 48-49 (2011), each of which is hereby incorporated by reference in its entirety).

In a further embodiment luminescent dye-labeled hybrid nanoparticles can be used. These nanoparticles consist of a SiO₂-based particle matrix with covalently attached organic fluorophores. They combine the optical properties of organic dye molecules and the inorganic particle matrix properties. As a result they show an increased resistance to photobleaching and a decreased dye leakage. Respective nanoparticles are disclosed by Probst (Expert Review of Molecular Diagnostics 12(1): 49-64 (2012), which is hereby incorporated by reference in its entirety).

In another embodiment, cadmium-free quantum dots can be used. These nanoparticles show bright emission in the visible and near infra-red region of the spectrum. Respective nanoparticles are developed by Nanoco Technologies Ltd. (Manchester, UK) and are disclosed in WO07/020416, WO08/100276, WO10/52455, WO10/15824, WO10/10329 and WO13/93631, each of which is hereby incorporated by reference in its entirety.

In one embodiment of the invention (group II-alloyed) group semiconductor quantum dots, group III-V quantum dots or micronized semiconductor nanocrystal complexes as developed by Evident Technologies (Troy, N.Y., USA) can be used. These nanoparticles are disclosed in WO07/118118, WO08/94292, WO06/17125 or WO05/110916, respectively, each of which is hereby incorporated by reference in its entirety.

In another embodiment superparamagnetic iron oxide nanoparticles (IONPs), block-copolymer micelles, nanocells, dendrimers, nanotubes, polymersomes and XPclad® nanoparticles can be used. Respective nanoparticles are disclosed by Singh and Lillard (Exp Mol Pathol, 86(3):215-223 (2009), which is hereby incorporated by reference in its entirety) and Xie et al. (Adv Drug Deliv Rev 62(11):1064-1079 (2010), which is hereby incorporated by reference in its entirety).

In a further embodiment of the invention non-Cd-nanoparticles can be used, comprising a core area being covered by a shell area which represents an antireflective coating of the core area. Respective nanoparticles are disclosed by US 2008/0286826 A1, which is hereby incorporated by reference in its entirety.

In another embodiment of the invention, magnetic particles can be used, which are especially suited for targeted drug delivery. In a preferred embodiment said magnetic particles consist of superparamagnetic metal oxides and/or metals and are coated with the peptides of the invention and optionally with one or more additional drugs. Respective magnetic particles are disclosed by EP 1 267 843 B1, which is hereby incorporated by reference in its entirety.

Another aspect of the invention relates to a pharmaceutical composition that includes the peptide, as described herein, in an aqueous medium.

In certain embodiments the peptides in the pharmaceutical composition form micelle structures. Additionally, the pharmaceutical composition can further contain a therapeutic agent encapsulated within the micelle structures. These therapeutic agents can be hydrophobic.

Exemplary therapeutic agents include, but are not limited to, the following: analgesics, anti-inflammatory agents, antihelminthics, anti-arrhythmic agents, anti-bacterial agents, anti-viral agents, anti-coagulants, anti-depressants, anti-diabetics, anti-epileptics, anti-fungal agents, anti-gout agents, anti-hypertensive agents, anti-thrombogenic agents, anti-claudication agents, anti-atherosclerotic drugs, vascular agents, anti-malarials, anti-migraine agents, anti-muscarinic agents, anti-neoplastic agents (e.g., antiproliferative or chemotherapeutic agents), erectile dysfunction improvement agents, immunosuppressants, anti-protozoal agents, anti-thyroid agents, anxiolytic agents, sedatives, hypnotics, neuroleptics, β-blockers, cardiac inotropic agents, corticosteroids, diuretics, anti-parkinsonian agents, gastro-intestinal agents, histamine receptor antagonists, keratolyptics, lipid regulating agents, anti-anginal agents, Cox-2 inhibitors, leukotriene inhibitors, macrolides, muscle relaxants, nutritional agents, opioid analgesics, protease inhibitors, sex hormones, stimulants, anti-osteoporosis agents, anti-obesity agents, cognition enhancers, anti-urinary incontinence agents, anti-benign prostate hypertrophy agents, essential fatty acids, non-essential fatty acids, antioxidants, and mixtures thereof.

Further non-limiting examples of the therapeutic agents in the pharmaceutical composition include: acetretin, albendazole, albuterol, aminoglutethimide, amiodarone, amlodipine, amphetamine, amphotericin B, arginine, atorvastatin, atovaquone, azithromycin, baclofen, beclomethasone, benazepril, benzonatate, betamethasone, bicalutanide, budesonide, bupropion, busulfan, butenafine, calcifediol, calcipotriene, calcitriol, camptothecin, candesartan, capsaicin, captopril, carbamezepine, carotenes, celecoxib, cerivastatin, cetirizine, chlorpheniramine, cholecalciferol, cilazepril, cilostazol, cimetidine, cinnarizine, ciprofloxacin, cisapride, clarithromycin, clemastine, clomiphene, clomipramine, clonidine, clopidogrel, codeine, coenzyme Q10, cyclobenzaprine, cyclosporin, danazol, dantrolene, dexchlorpheniramine, diclofenac, dicoumarol, digoxin, dehydroepiandrosterone, dihydroergotamine, dihydrotachysterol, dirithromycin, donezepil, doxazosin, efavirenz, eprosartan, ergocalciferol, ergotamine, essential fatty acid sources, etodolac, etoposide, famotidine, fenofibrate, fentanyl, fexofenadine, finasteride, fluconazole, flurbiprofen, fluvastatin, fosphenyloin, frovatriptan, fuirazolidone, gabapentin, gemfibrozil, glibenclamide, glipizide, glyburide, glimepiride, griseofulvin, halofantrine, ibuprofen, irbesartan, irinotecan, isosorbide dinitrate, isotretinoin, itraconazole, ivermectin, ketenserin, ketoconazole, ketorolac, lamotrigine, lansoprazole, leflunomide, lisinopril, loperamide, loratadine, losartan, lovastatin, L-thryroxine, lutein, lycopene, medroxyprogesterone, mifepristone, mefloquine, megestrol acetate, methadone, methoxsalen, methyldopa, metronidazole, miconazole, midazolam, miglitol, minoxidil, mitoxantrone, montelukast, moxonidine, nabumetone, nalbuphine, naratriptan, nelfinavir, nifedipine, nil solidipine, nilutanide, nitrofurantoin, nitroglycerin, nizatidine, omeprazole, oprevelkin, oestradiol, oxaprozin, paclitaxel, paracalcitol, paroxetine, pentazocine, pioglitazone, pizofetin, prazosin, pravastatin, prednisolone, probucol, progesterone, pseudoephedrine, pyridostigmine, rabeprazole, raloxifene, rofecoxib, repaglinide, rifabutine, rifapentine, rimexolone, ritanovir, rizatriptan, rosiglitazone, saquinavir, sertraline, sibutramine, sildenafil citrate, simvastatin, sirolimus, spironolactone, sumatriptan, tacrine, tacrolimus, tamoxifen, tamsulosin, targretin, tazarotene, telmisartan, teniposide, terbinafine, terazosin, tetrahydrocannabinol, tiagabine, ticlopidine, tirofibran, tizanidine, topiramate, topotecan, toremitfene, tramadol, tretinoin, troglitazone, trovafloxacin, ubidecarenone, urapidil, valsartan, venlafaxine, verteporfin, vigabatrin, vitamin A, vitamin D, vitamin E, vitamin K, zafirlukast, zileuton, zolmitriptan, zolpidem, zopiclone, pharmaceutically acceptable salts, isomers, and derivatives thereof, and mixtures thereof.

In certain aspects of the invention, the therapeutic agent in the pharmaceutical composition is an antiproliferative or chemotherapeutic drug. Exemplary antiproliferative or chemotherapeutic drugs include, but are not limited to, Abarelix, aldesleukin, Aldesleukin, Alemtuzumab, Alitretinoin, Allopurinol, Altretamine, Amifostine, Anastrozole, Arsenic trioxide, Asparaginase, Azacitidine, β-lapachone, BCG Live, Bevacuzimab, Avastin, Fluorouracil, Bexarotene, Bleomycin, Bortezomib, Busulfan, Calusterone, Capecitabine, Camptothecin, Carboplatin, Carmustine, Celecoxib, Cetuximab, Chlorambucil, Cisplatin, Cladribine, Clofarabine, Cyclophosphamide, Cytarabine, Dactinomycin, Darbepoetin alfa, Daunorubicin, Denileukin, Dexrazoxane, Docetaxel, Doxorubicin (neutral), Doxorubicin hydrochloride, Dromostanolone Propionate, Epirubicin, Epoetin alfa, Erlotinib, Estramustine, Etoposide Phosphate, Etoposide, Exemestane, Filgrastim, floxuridine fludarabine, Fulvestrant, Gefitinib, Gemcitabine, Gemtuzumab, Goserelin Acetate, Histrelin Acetate, Hydroxyurea, Ibritumomab, Idarubicin, Ifosfamide, Imatinib Mesylate, Interferon Alfa-2a, Interferon Alfa-2b, Irinotecan, Lenalidomide, Letrozole, Leucovorin, Leuprolide Acetate, Levamisole, Lomustine, Megestrol Acetate, Melphalan, Mercaptopurine, 6-MP, Mesna, Methotrexate, Methoxsalen, Mitomycin C, Mitotane, Mitoxantrone, Nandrolone, Nelarabine, Nofetumomab, Oprelvekin, Oxaliplatin, Paclitaxel, Palifermin, Pamidronate, Pegademase, Pegaspargase, Pegfilgrastim, Pemetrexed Disodium, Pentostatin, Pipobroman, Plicamycin, Porfimer Sodium, Procarbazine, Quinacrine, Rasburicase, Rituximab, Sargramostim, Sorafenib, Streptozocin, Sunitinib Maleate, Talc, Tamoxifen, Temozolomide, Teniposide, VM-26, Testolactone, Thioguanine, 6-TG, Thiotepa, Topotecan, Toremifene, Tositumomab, Trastuzumab, Tretinoin, ATRA, Uracil Mustard, Valrubicin, Vinblastine, Vincristine, Vinorelbine, Zoledronate, and Zoledronic acid.

In certain embodiments the therapeutic agent of the pharmaceutical composition is a protein or polypeptide. The protein or polypeptide can be a cytokine or growth factor (such as VEGF, FGF, MCP-1, PIGF, KGF, PDGF), or an antibody or binding portion thereof.

In certain embodiments, the therapeutic agent of the pharmaceutical composition is a treatment for Alzheimer's Disease or Parkinson's Disease. Exemplary therapeutic agents for the treatment of these diseases include, without limitation, a cholinesterase inhibitor, an N-methyl D-aspartate (NMDA) antagonist, L-Dopa, a dopamine agonist, an MAO B inhibitor, a COMT inhibitor, an anticholinergic, or amantadine.

In a further embodiment, the therapeutic agent of the pharmaceutical composition is an antioxidant, a coenzyme, a vitamin, a metabolite, or a mineral.

In certain embodiments, the peptide of the pharmaceutical composition is present at a concentration of from about 100 nM to about 500 μM. Furthermore, the therapeutic agent can be present in an amount from about 1 nM to about 10 μM. Single doses of the pharmaceutical composition may contain from 0.1 μg to 0.1 g of the peptide, preferably 4 μg to 0.04 g or 400 μg to 0.4 g; and any effective amount of the therapeutic agent. Typically, single doses of the therapeutic agent range from 1 μg/kg·body weight to 1000 mg/kg·body weight (although lesser or greater dosages are also contemplated).

In some embodiments, the carrier is an aqueous medium. In one embodiment, the aqueous medium is a sterile isotonic aqueous buffer, which is typically well tolerated for administration to an individual. Additional exemplary aqueous media include, without limitation, normal saline (about 0.9% NaCl), phosphate buffered saline (“PBS”), sterile water/distilled autoclaved water (“DAW”), as well as cell growth medium (e.g., MEM, with or without serum), aqueous solutions of dimethyl sulfoxide (“DMSO”), polyethylene glycol (“PEG”), and/or dextran (less than 6% per by weight).

To improve patient tolerance to administration, the pharmaceutical composition may have a pH of about 4.5 to about 8.5. In some embodiments, sodium hydroxide or hydrochloric is added to the pharmaceutical composition to adjust the pH.

In other embodiments, the pharmaceutical composition includes a weak acid or salt as a buffering agent to maintain pH. Citric acid has the ability to chelate divalent cations and can thus also prevent oxidation, thereby serving two functions as both a buffering agent and an antioxidant stabilizing agent. Citric acid is typically used in the form of a sodium salt, typically 10-500 mM. Other weak acids or their salts can also be used.

The pharmaceutical composition may also include solubilizing agents, preservatives, stabilizers, emulsifiers, and the like. A local anesthetic (e.g., lidocaine, benzocaine, etc.) may also be included in the compositions, particularly for injectable forms, to ease pain at the site of the injection.

Another aspect of the present invention relates to enzymatic (e.g., phosphatase) cleavage products of the peptides, whereby cleavage of the thiophosphate group disrupts the ability of the (resulting cleaved) peptide to form micelle structures. Instead, given the loss of the phosphate group from the C-terminus of the peptide, the resulting product is capable of self-assembly to form nanofibers and possibly larger hydrogel assemblies containing those nanofibers.

Exemplary cleavage products include, without limitation, the following peptides:
Naphthyl-(CH₂)—C(O)—NH—FF—C(O)—NH(CH₂)₂SH;
NBD-(CH₂)₂—C(O)—NH—FF—C(O)—NH(CH₂)₂SH;
Naphthyl-(CH₂)—C(O)—NH-ff-C(O)—NH(CH₂)₂SH;
NBD-(CH₂)₂—C(O)—NH-ff-C(O)—NH(CH₂)₂SH;
Naphthyl-(CH₂)—C(O)—NH—FFK—C(O)—NH(CH₂)₂SH;
NBD-(CH₂)₂—C(O)—NH—FFK—C(O)—NH(CH₂)₂SH;
Naphthyl-(CH₂)—C(O)—NH-ffk-C(O)—NH(CH₂)₂SH;
NBD-(CH₂)₂—C(O)—NH-ffk-C(O)—NH(CH₂)₂SH;
Naphthyl-(CH₂)—C(O)—NH—FFKY—C(O)—NH(CH₂)₂SH;
NBD-(CH₂)₂—C(O)—NH—FFKY—C(O)—NH(CH₂)₂SH;
Naphthyl-(CH₂)—C(O)—NH-ffky-C(O)—NH(CH₂)₂SH;
NBD-(CH₂)₂—C(O)—NH-ffky-C(O)—NH(CH₂)₂SH;
Naphthyl-(CH₂)—C(O)—NH—FFFKY—C(O)—NH(CH₂)₂SH;
NBD-(CH₂)₂—C(O)—NH—FFFKY—C(O)—NH(CH₂)₂SH;
Naphthyl-(CH₂)—C(O)—NH-fffky-C(O)—NH(CH₂)₂SH;
NBD-(CH₂)₂—C(O)—NH-fffky-C(O)—NH(CH₂)₂SH;
Naphthyl-(CH₂)—C(O)—NH—FFGKY—C(O)—NH(CH₂)₂SH;
NBD-(CH₂)₂—C(O)—NH—FFGKY—C(O)—NH(CH₂)₂SH;
Naphthyl-(CH₂)—C(O)—NH-ffgky-C(O)—NH(CH₂)₂SH;
NBD-(CH₂)₂—C(O)—NH-ffgky-C(O)—NH(CH₂)₂SH;
Naphthyl-(CH₂)—C(O)—NH—FFGK—C(O)—NH(CH₂)₂SH;
NBD-(CH₂)₂—C(O)—NH—FFGK—C(O)—NH(CH₂)₂SH;
Naphthyl-(CH₂)—C(O)—NH-ffgk-C(O)—NH(CH₂)₂SH;
NBD-(CH₂)₂—C(O)—NH-ffgk-C(O)—NH(CH₂)₂SH;
Naphthyl-(CH₂)—C(O)—NH—FFGKF—C(O)—NH(CH₂)₂SH;
NBD-(CH₂)₂—C(O)—NH—FFGKF—C(O)—NH(CH₂)₂SH;
Naphthyl-(CH₂)—C(O)—NH-ffgkf-C(O)—NH(CH₂)₂SH;
NBD-(CH₂)₂—C(O)—NH-ffgkf-C(O)—NH(CH₂)₂SH;
Naphthyl-(CH₂)—C(O)—NH—FFK(Dmt)-C(O)—NH(CH₂)₂SH;
NBD-(CH₂)₂—C(O)—NH—FFK(Dmt)-C(O)—NH(CH₂)₂SH;
Naphthyl-(CH₂)—C(O)—NH-ffk(dmt)-C(O)—NH(CH₂)₂SH;
NBD-(CH₂)₂—C(O)—NH-ffk(dmt)-C(O)—NH(CH₂)₂SH;
Naphthyl-(CH₂)—C(O)—NH—FFFK(Dmt)-C(O)—NH(CH₂)₂SH;
NBD-(CH₂)₂—C(O)—NH—FFFK(Dmt)-C(O)—NH(CH₂)₂SH;
Naphthyl-(CH₂)—C(O)—NH-fffk(dmt)-C(O)—NH(CH₂)₂SH;
NBD-(CH₂)₂—C(O)—NH-fffk(dmt)-C(O)—NH(CH₂)₂SH;
Naphthyl-(CH₂)—C(O)—NH—FFGK(Dmt)-C(O)—NH(CH₂)₂SH;
NBD-(CH₂)₂—C(O)—NH—FFGK(Dmt)-C(O)—NH(CH₂)₂SH;
Naphthyl-(CH₂)—C(O)—NH-ffgk(dmt)—C(O)—NH(CH₂)₂SH;
NBD-(CH₂)₂—C(O)—NH-ffgk(dmt)-C(O)—NH(CH₂)₂SH;
Naphthyl-(CH₂)—C(O)—NH—FFCY—C(O)—NH(CH₂)₂SH;
NBD-(CH₂)₂—C(O)—NH—FFCY—C(O)—NH(CH₂)₂SH;
Naphthyl-(CH₂)—C(O)—NH-ffcy-C(O)—NH(CH₂)₂SH;
NBD-(CH₂)₂—C(O)—NH-ffcy-C(O)—NH(CH₂)₂SH;
Naphthyl-(CH₂)—C(O)—NH—FFFCY—C(O)—NH(CH₂)₂SH;
NBD-(CH₂)₂—C(O)—NH—FFFCY—C(O)—NH(CH₂)₂SH;
Naphthyl-(CH₂)—C(O)—NH-fffcy-C(O)—NH(CH₂)₂SH;
NBD-(CH₂)₂—C(O)—NH-fffcy-C(O)—NH(CH₂)₂SH;
Naphthyl-(CH₂)—C(O)—NH—FFGCY—C(O)—NH(CH₂)₂SH;
NBD-(CH₂)₂—C(O)—NH—FFGCY—C(O)—NH(CH₂)₂SH;
Naphthyl-(CH₂)—C(O)—NH-ffgcy-C(O)—NH(CH₂)₂SH;
NBD-(CH₂)₂—C(O)—NH-ffgcy-C(O)—NH(CH₂)₂SH;
Naphthyl-(CH₂)—C(O)—NH—FFGC—C(O)—NH(CH₂)₂SH;
NBD-(CH₂)₂—C(O)—NH—FFGC—C(O)—NH(CH₂)₂SH;
Naphthyl-(CH₂)—C(O)—NH-ffgc-C(O)—NH(CH₂)₂SH;
NBD-(CH₂)₂—C(O)—NH-ffgc-C(O)—NH(CH₂)₂SH;
Naphthyl-(CH₂)—C(O)—NH—FFGCF—C(O)—NH(CH₂)₂SH;
NBD-(CH₂)₂—C(O)—NH—FFGCF—C(O)—NH(CH₂)₂SH;
Naphthyl-(CH₂)—C(O)—NH-ffgcf-C(O)—NH(CH₂)₂SH;
NBD-(CH₂)₂—C(O)—NH-ffgcf-C(O)—NH(CH₂)₂SH;
Naphthyl-(CH₂)—C(O)—NH—FFC(Dmt)-C(O)—NH(CH₂)₂SH;
NBD-(CH₂)₂—C(O)—NH—FFC(Dmt)-C(O)—NH(CH₂)₂SH;
Naphthyl-(CH₂)—C(O)—NH-ffc(dmt)-C(O)—NH(CH₂)₂SH;
NBD-(CH₂)₂—C(O)—NH-ffc(dmt)-C(O)—NH(CH₂)₂SH;
Naphthyl-(CH₂)—C(O)—NH—FFFC(Dmt)-C(O)—NH(CH₂)₂SH;
NBD-(CH₂)₂—C(O)—NH—FFFC(Dmt)-C(O)—NH(CH₂)₂SH;
Naphthyl-(CH₂)—C(O)—NH-fffc(dmt)-C(O)—NH(CH₂)₂SH;
NBD-(CH₂)₂—C(O)—NH-fffc(dmt)-C(O)—NH(CH₂)₂SH;
Naphthyl-(CH₂)—C(O)—NH—FFGC (Dmt)-C(O)—NH(CH₂)₂SH;
NBD-(CH₂)₂—C(O)—NH—FFGC(Dmt)-C(O)—NH(CH₂)₂SH;
Naphthyl-(CH₂)—C(O)—NH-ffgc(dmt)-C(O)—NH(CH₂)₂SH;
NBD-(CH₂)₂—C(O)—NH-ffgc(dmt)-C(O)—NH(CH₂)₂SH;
Naphthyl-CH₂C (O)—NH—FFK(ε-Z₂)—C(O)—NH(CH₂)₂SH;
Naphthyl-CH₂C (O)—NH-ffk(ε-Z₂)—C(O)—NH(CH₂)₂SH;
Naphthyl-CH₂C(O)—NH-FFK(ε-Z₂)Y—C(O)—NH(CH₂)₂SH;
Naphthyl-CH₂C(O)—NH-ffk(ε-Z₂)y-C(O)—NH(CH₂)₂SH;
Naphthyl-CH₂C(O)—NH—FFFK(ε-Z₂)Y—C(O)—NH(CH₂)₂SH;
Naphthyl-CH₂C(O)—NH-fffk(ε-Z₂)y-C(O)—NH(CH₂)₂SH;
Naphthyl-CH₂C(O)—NH—FFGK(ε-Z₂)Y—C(O)—NH(CH₂)₂SH;
Naphthyl-CH₂C(O)—NH-ffgk(ε-Z₂)y-C(O)—NH(CH₂)₂SH;
Naphthyl-CH₂C(O)—NH—FFGK(ε-Z₂)—C(O)—NH(CH₂)₂SH;
Naphthyl-CH₂C(O)—NH-ffgk(ε-Z₂)—C(O)—NH(CH₂)₂SH;
Naphthyl-CH₂C(O)—NH—FFGK(ε-Z₂)F—C(O)—NH(CH₂)₂SH;
Naphthyl-CH₂C(O)—NH-ffgk(ε-Z₂)f-C(O)—NH(CH₂)₂SH;
Naphthyl-CH₂C(O)—NH—FFK(ε-Z₂)(Dmt)-C(O)—NH(CH₂)₂SH;
Naphthyl-CH₂C(O)—NH-ffk(ε-Z₂)(dmt)-C(O)—NH(CH₂)₂SH;
Naphthyl-CH₂C(O)—NH—FFFK(ε-Z₂)(Dmt)-C(O)—NH(CH₂)₂SH;
Naphthyl-CH₂C(O)—NH-fffk(ε-Z₂)(dmt)-C(O)—NH(CH₂)₂SH;
Naphthyl-CH₂C(O)—NH—FFGK(ε-Z₂)(Dmt)-C(O)—NH(CH₂)₂SH;
Naphthyl-CH₂C(O)—NH-ffgk(ε-Z₂)(dmt)-C(O)—NH(CH₂)₂SH;
NBD-(CH₂)₂—C(O)—NH—FFK(ε-Z₂)—C(O)—NH(CH₂)₂SH;
NBD-(CH₂)₂—C(O)—NH-ffk(ε-Z₂)—C(O)—NH(CH₂)₂SH;
NBD-(CH₂)₂—C(O)—NH—FFK(ε-Z₂)Y—C(O)—NH(CH₂)₂SH;
NBD-(CH₂)₂—C(O)—NH-ffk(ε-Z₂)y-C(O)—NH(CH₂)₂SH;
NBD-(CH₂)₂—C(O)—NH—FFFK(ε-Z₂)Y—C(O)—NH(CH₂)₂SH;
NBD-(CH₂)₂—C(O)—NH-fffk(ε-Z₂)y-C(O)—NH(CH₂)₂SH;
NBD-(CH₂)₂—C(O)—NH—FFGK(ε-Z₂)Y—C(O)—NH(CH₂)₂SH;
NBD-(CH₂)₂—C(O)—NH-ffgk(ε-Z₂)y-C(O)—NH(CH₂)₂SH;
NBD-(CH₂)₂—C(O)—NH—FFGK(ε-Z₂)—C(O)—NH(CH₂)₂SH;
NBD-(CH₂)₂—C(O)—NH-ffgk(ε-Z₂)—C(O)—NH(CH₂)₂SH;
NBD-(CH₂)₂—C(O)—NH—FFGK(ε-Z₂)F—C(O)—NH(CH₂)₂SH;
NBD-(CH₂)₂—C(O)—NH-ffgk(ε-Z₂)f-C(O)—NH(CH₂)₂SH;
NBD-(CH₂)₂—C(O)—NH—FFK(ε-Z₂)(Dmt)-C(O)—NH(CH₂)₂SH;
NBD-(CH₂)₂—C(O)—NH-ffk(ε-Z₂)(dmt)-C(O)—NH(CH₂)₂SH;
NBD-(CH₂)₂—C(O)—NH—FFFK(ε-Z₂)(Dmt)-C(O)—NH(CH₂)₂SH;
NBD-(CH₂)₂—C(O)—NH-fffk(ε-Z₂)(dmt)-C(O)—NH(CH₂)₂SH;
NBD-(CH₂)₂—C(O)—NH—FFGK(ε-Z₂)(Dmt)-C(O)—NH(CH₂)₂SH;
NBD-(CH₂)₂—C(O)—NH-ffgk(ε-Z₂)(dmt)-C(O)—NH(CH₂)₂SH;
Naphthyl-(CH₂)—C(O)—NH—FF—C(O)—NH(CH₂)₃NH(CH₂)₂SH;
NBD-(CH₂)₂—C(O)—NH—FF—C(O)—NH(CH₂)₃NH(CH₂)₂SH;
Naphthyl-(CH₂)—C(O)—NH-ff-C(O)—NH(CH₂)₃NH(CH₂)₂SH;
NBD-(CH₂)₂—C(O)—NH-ff-C(O)—NH(CH₂)₃NH(CH₂)₂SH;
Naphthyl-(CH₂)—C(O)—NH—FFK—C(O)—NH(CH₂)₃NH(CH₂)₂SH;
NBD-(CH₂)₂—C(O)—NH—FFK—C(O)—NH(CH₂)₃NH(CH₂)₂SH;
Naphthyl-(CH₂)—C(O)—NH-ffk-C(O)—NH(CH₂)₃NH(CH₂)₂SH;
NBD-(CH₂)₂—C(O)—NH-ffk-C(O)—NH(CH₂)₃NH(CH₂)₂SH;
Naphthyl-(CH₂)—C(O)—NH—FFKY—C(O)—NH(CH₂)₃NH(CH₂)₂SH;
NBD-(CH₂)₂—C(O)—NH—FFKY—C(O)—NH(CH₂)₃NH(CH₂)₂SH;
Naphthyl-(CH₂)—C(O)—NH-ffky-C(O)—NH(CH₂)₃NH(CH₂)₂SH;
NBD-(CH₂)₂—C(O)—NH-ffky-C(O)—NH(CH₂)₃NH(CH₂)₂SH;
Naphthyl-(CH₂)—C(O)—NH—FFFKY—C(O)—NH(CH₂)₃NH(CH₂)₂SH;
NBD-(CH₂)₂—C(O)—NH—FFFKY—C(O)—NH(CH₂)₃NH(CH₂)₂SH;
Naphthyl-(CH₂)—C(O)—NH-fffky-C(O)—NH(CH₂)₃NH(CH₂)₂SH;
NBD-(CH₂)₂—C(O)—NH-fffky-C(O)—NH(CH₂)₃NH(CH₂)₂SH;
Naphthyl-(CH₂)—C(O)—NH—FFGKY—C(O)—NH(CH₂)₃NH(CH₂)₂SH;
NBD-(CH₂)₂—C(O)—NH—FFGKY—C(O)—NH(CH₂)₃NH(CH₂)₂SH;
Naphthyl-(CH₂)—C(O)—NH-ffgky-C(O)—NH(CH₂)₃NH(CH₂)₂SH;
NBD-(CH₂)₂—C(O)—NH-ffgky-C(O)—NH(CH₂)₃NH(CH₂)₂SH;
Naphthyl-(CH₂)—C(O)—NH—FFGK—C(O)—NH(CH₂)₃NH(CH₂)₂SH;
NBD-(CH₂)₂—C(O)—NH—FFGK—C(O)—NH(CH₂)₃NH(CH₂)₂SH;
Naphthyl-(CH₂)—C(O)—NH-ffgk-C(O)—NH(CH₂)₃NH(CH₂)₂SH;
NBD-(CH₂)₂—C(O)—NH-ffgk-C(O)—NH(CH₂)₃NH(CH₂)₂SH;
Naphthyl-(CH₂)—C(O)—NH—FFGKF—C(O)—NH(CH₂)₃NH(CH₂)₂SH;
NBD-(CH₂)₂—C(O)—NH—FFGKF—C(O)—NH(CH₂)₃NH(CH₂)₂SH;
Naphthyl-(CH₂)—C(O)—NH-ffgkf-C(O)—NH(CH₂)₃NH(CH₂)₂SH;
NBD-(CH₂)₂—C(O)—NH-ffgkf-C(O)—NH(CH₂)₃NH(CH₂)₂SH;
Naphthyl-(CH₂)—C(O)—NH—FFK(Dmt)-C(O)—NH(CH₂)₃NH(CH₂)₂SH;
NBD-(CH₂)₂—C(O)—NH—FFK(Dmt)-C(O)—NH(CH₂)₃NH(CH₂)₂SH;
Naphthyl-(CH₂)—C(O)—NH-ffk(dmt)-C(O)—NH(CH₂)₃NH(CH₂)₂SH;
NBD-(CH₂)₂—C(O)—NH-ffk(dmt)-C(O)—NH(CH₂)₃NH(CH₂)₂SH;
Naphthyl-(CH₂)—C(O)—NH—FFFK(Dmt)-C(O)—NH(CH₂)₃NH(CH₂)₂SH;
NBD-(CH₂)₂—C(O)—NH—FFFK(Dmt)-C(O)—NH(CH₂)₃NH(CH₂)₂SH;
Naphthyl-(CH₂)—C(O)—NH-fffk(dmt)-C(O)—NH(CH₂)₃NH(CH₂)₂SH;
NBD-(CH₂)₂—C(O)—NH-fffk(dmt)—C(O)—NH(CH₂)₃NH(CH₂)₂SH;
Naphthyl-(CH₂)—C(O)—NH—FFGK(Dmt)-C(O)—NH(CH₂)₃NH(CH₂)₂SH;
NBD-(CH₂)₂—C(O)—NH—FFGK(Dmt)-C(O)—NH(CH₂)₃NH(CH₂)₂SH;
Naphthyl-(CH₂)—C(O)—NH-ffgk(dmt)—C(O)—NH(CH₂)₃NH(CH₂)₂SH;
NBD-(CH₂)₂—C(O)—NH-ffgk(dmt)—C(O)—NH(CH₂)₃NH(CH₂)₂SH;
Naphthyl-(CH₂)—C(O)—NH—FFCY—C(O)—NH(CH₂)₃NH(CH₂)₂SH;
NBD-(CH₂)₂—C(O)—NH—FFCY—C(O)—NH(CH₂)₃NH(CH₂)₂SH;
Naphthyl-(CH₂)—C(O)—NH-ffcy-C(O)—NH(CH₂)₃NH(CH₂)₂SH;
NBD-(CH₂)₂—C(O)—NH-ffcy-C(O)—NH(CH₂)₃NH(CH₂)₂SH;
Naphthyl-(CH₂)—C(O)—NH—FFFCY—C(O)—NH(CH₂)₃NH(CH₂)₂SH;
NBD-(CH₂)₂—C(O)—NH—FFFCY—C(O)—NH(CH₂)₃NH(CH₂)₂SH;
Naphthyl-(CH₂)—C(O)—NH-fffcy-C(O)—NH(CH₂)₃NH(CH₂)₂SH;
NBD-(CH₂)₂—C(O)—NH-fffcy-C(O)—NH(CH₂)₃NH(CH₂)₂SH;
Naphthyl-(CH₂)—C(O)—NH—FFGCY—C(O)—NH(CH₂)₃NH(CH₂)₂SH;
NBD-(CH₂)₂—C(O)—NH—FFGCY—C(O)—NH(CH₂)₃NH(CH₂)₂SH;
Naphthyl-(CH₂)—C(O)—NH-ffgcy-C(O)—NH(CH₂)₃NH(CH₂)₂SH;
NBD-(CH₂)₂—C(O)—NH-ffgcy-C(O)—NH(CH₂)₃NH(CH₂)₂SH;
Naphthyl-(CH₂)—C(O)—NH—FFGC—C(O)—NH(CH₂)₃NH(CH₂)₂SH;
NBD-(CH₂)₂—C(O)—NH—FFGC—C(O)—NH(CH₂)₃NH(CH₂)₂SH;
Naphthyl-(CH₂)—C(O)—NH-ffgc-C(O)—NH(CH₂)₃NH(CH₂)₂SH;
NBD-(CH₂)₂—C(O)—NH-ffgc-C(O)—NH(CH₂)₃NH(CH₂)₂SH;
Naphthyl-(CH₂)—C(O)—NH—FFGCF—C(O)—NH(CH₂)₃NH(CH₂)₂SH;
NBD-(CH₂)₂—C(O)—NH—FFGCF—C(O)—NH(CH₂)₃NH(CH₂)₂SH;
Naphthyl-(CH₂)—C(O)—NH-ffgcf-C(O)—NH(CH₂)₃NH(CH₂)₂SH;
NBD-(CH₂)₂—C(O)—NH-ffgcf-C(O)—NH(CH₂)₃NH(CH₂)₂SH;
Naphthyl-(CH₂)—C(O)—NH—FFC(Dmt)-C(O)—NH(CH₂)₃NH(CH₂)₂SH;
NBD-(CH₂)₂—C(O)—NH—FFC(Dmt)-C(O)—NH(CH₂)₃NH(CH₂)₂SH;
Naphthyl-(CH₂)—C(O)—NH-ffc(dmt)-C(O)—NH(CH₂)₃NH(CH₂)₂SH;
NBD-(CH₂)₂—C(O)—NH-ffc(dmt)-C(O)—NH(CH₂)₃NH(CH₂)₂SH;
Naphthyl-(CH₂)—C(O)—NH—FFFC(Dmt)-C(O)—NH(CH₂)₃NH(CH₂)₂SH;
NBD-(CH₂)₂—C(O)—NH—FFFC(Dmt)-C(O)—NH(CH₂)₃NH(CH₂)₂SH;
Naphthyl-(CH₂)—C(O)—NH-fffc(dmt)-C(O)—NH(CH₂)₃NH(CH₂)₂SH;
NBD-(CH₂)₂—C(O)—NH-fffc(dmt)-C(O)—NH(CH₂)₃NH(CH₂)₂SH;
Naphthyl-(CH₂)—C(O)—NH—FFGC(Dmt)-C(O)—NH(CH₂)₃NH(CH₂)₂SH;
NBD-(CH₂)₂—C(O)—NH—FFGC(Dmt)-C(O)—NH(CH₂)₃NH(CH₂)₂SH;
Naphthyl—(CH₂)—C(O)—NH-ffgc(dmt)-C(O)—NH(CH₂)₃NH(CH₂)₂SH;
NBD-(CH₂)₂—C(O)—NH-ffgc(dmt)-C(O)—NH(CH₂)₃NH(CH₂)₂SH;
Naphthyl-CH₂C(O)—NH—FFK(ε-Z₂)—C(O)—NH(CH₂)₃NH(CH₂)₂SH;
Naphthyl-CH₂C(O)—NH-ffk(ε-Z₂)—C(O)—NH(CH₂)₃NH(CH₂)₂SH;
Naphthyl-CH₂C(O)—NH—FFK(ε-Z₂)Y—C(O)—NH(CH₂)₃NH(CH₂)₂SH;
Naphthyl-CH₂C(O)—NH-ffk(ε-Z₂)y-C(O)—NH(CH₂)₃NH(CH₂)₂SH;
Naphthyl-CH₂C(O)—NH—FFFK(ε-Z₂)Y—C(O)—NH(CH₂)₃NH(CH₂)₂SH;
Naphthyl-CH₂C(O)—NH-fffk(ε-Z₂)y-C(O)—NH(CH₂)₃NH(CH₂)₂SH;
Naphthyl-CH₂C(O)—NH—FFGK(ε-Z₂)Y—C(O)—NH(CH₂)₃NH(CH₂)₂SH;
Naphthyl-CH₂C(O)—NH-ffgk(ε-Z₂)y-C(O)—NH(CH₂)₃NH(CH₂)₂SH;
Naphthyl-CH₂C(O)—NH—FFGK(ε-Z₂)—C(O)—NH(CH₂)₃NH(CH₂)₂SH;
Naphthyl-CH₂C(O)—NH-ffgk(ε-Z₂)—C(O)—NH(CH₂)₃NH(CH₂)₂SH;
Naphthyl-CH₂C(O)—NH—FFGK(ε-Z₂)F—C(O)—NH(CH₂)₃NH(CH₂)₂SH;
Naphthyl-CH₂C(O)—NH-ffgk(ε-Z₂)f-C(O)—NH(CH₂)₃NH(CH₂)₂SH;
Naphthyl-CH₂C(O)—NH—FFK(ε-Z₂)(Dmt)-C(O)—NH(CH₂)₃NH(CH₂)₂SH;
Naphthyl-CH₂C(O)—NH-ffk(ε-Z₂)(dmt)-C(O)—NH(CH₂)₃NH(CH₂)₂SH;
Naphthyl-CH₂C(O)—NH—FFFK(ε-Z₂)(Dmt)-C(O)—NH(CH₂)₃NH(CH₂)₂SH;
Naphthyl-CH₂C(O)—NH-fffk(ε-Z₂)(dmt)-C(O)—NH(CH₂)₃NH(CH₂)₂SH;
Naphthyl-CH₂C(O)—NH—FFGK(ε-Z₂)(Dmt)-C(O)—NH(CH₂)₃NH(CH₂)₂SH;
Naphthyl-CH₂C(O)—NH-ffgk(ε-Z₂)(dmt)- C(O)—NH(CH₂)₃NH(CH₂)₂SH;
NBD-(CH₂)₂—C(O)—NH—FFK(ε-Z₂)—C(O)—NH(CH₂)₃NH(CH₂)₂SH;
NBD-(CH₂)₂—C(O)—NH-ffk(ε-Z₂)—C(O)—NH(CH₂)₃NH(CH₂)₂SH;
NBD-(CH₂)₂—C(O)—NH—FFK(ε-Z₂)Y—C(O)—NH(CH₂)₃NH(CH₂)₂SH;
NBD-(CH₂)₂—C(O)—NH-ffk(ε-Z₂)y-C(O)—NH(CH₂)₃NH(CH₂)₂SH;
NBD-(CH₂)₂—C(O)—NH—FFFK(ε-Z₂)Y—C(O)—NH(CH₂)₃NH(CH₂)₂SH;
NBD-(CH₂)₂—C(O)—NH-fffk(ε-Z₂)y-C(O)—NH(CH₂)₃NH(CH₂)₂SH;
NBD-(CH₂)₂—C(O)—NH—FFGK(ε-Z₂)Y—C(O)—NH(CH₂)₃NH(CH₂)₂SH;
NBD-(CH₂)₂—C(O)—NH-ffgk(ε-Z₂)y-C(O)—NH(CH₂)₃NH(CH₂)₂SH;
NBD-(CH₂)₂—C(O)—NH—FFGK(ε-Z₂)—C(O)—NH(CH₂)₃NH(CH₂)₂SH;
NBD-(CH₂)₂—C(O)—NH-ffgk(ε-Z₂)—C(O)—NH(CH₂)₃NH(CH₂)₂SH;
NBD-(CH₂)₂—C(O)—NH—FFGK(ε-Z₂)F—C(O)—NH(CH₂)₃NH(CH₂)₂SH;
NBD-(CH₂)₂—C(O)—NH-ffgk(ε-Z₂)f-C(O)—NH(CH₂)₃NH(CH₂)₂SH;
NBD-(CH₂)₂—C(O)—NH—FFK(ε-Z₂)(Dmt)-C(O)—NH(CH₂)₃NH(CH₂)₂SH;
NBD-(CH₂)₂—C(O)—NH-ffk(ε-Z₂)(dmt)-C(O)—NH(CH₂)₃NH(CH₂)₂SH;
NBD-(CH₂)₂—C(O)—NH—FFFK(ε-Z₂)(Dmt)-C(O)—NH(CH₂)₃NH(CH₂)₂SH;
NBD-(CH₂)₂—C(O)—NH-fffk(ε-Z₂)(dmt)-C(O)—NH(CH₂)₃NH(CH₂)₂SH;
NBD-(CH₂)₂—C(O)—NH—FFGK(ε-Z₂)(Dmt)-C(O)—NH(CH₂)₃NH(CH₂)₂SH;
NBD-(CH₂)₂—C(O)—NH-ffgk(ε-Z₂)(dmt)-C(O)—NH(CH₂)₃NH(CH₂)₂SH;
   wherein Dmt is 2,6-dimethyl-L-tyrosine; dmt is 2,6-dimethyl-D-tyrosine; NBD is 4-nitro-2,1,3-benzoxadiazolyl; and Z₂ is defined above. NBD-(CH₂)₂—C(O) denotes NBD-β-Ala.

Thus, in one embodiment, the invention relates to a nanofiber formed in an aqueous medium which includes self-assembled, enzymatically modified forms of the peptide as described herein. As used herein, the term “nanofiber” is defined as a fiber of material having any shape wherein at least one dimension, e.g. the diameter, width, thickness, and the like, is about 100 nm or less. Nanofiber diameters may be about 50 nm or less, about 40 nm or less, about 30 nm or less, about 20 nm or less, about 10 nm or less, about 5 nm or less, about 4 nm or less, about 3 nm or less, about 2 nm or less, or about 1 nm or less in diameter. Although the peptides of the present invention, upon self-assembly, as described herein, form nanofibers, persons of skill in the art should appreciate that such peptides may also form microfibrils that are larger than 100 nm thick.

In another embodiment, the invention relates to a supramolecular hydrogel formed in an aqueous medium that includes a self-assembled, enzymatically modified form of the peptide.

In these embodiments, the nanofiber or supramolecular hydrogel includes the enzymatically modified form of the peptide as described above, but the nanofiber or supramolecular hydrogel may optionally include an enzymatically unmodified form of the peptide, which may be incorporated into the same.

In addition, where combinations of peptides are present in the pharmaceutical composition, then nanofibers and supramolecular hydrogels formed by the enzymatically modified forms of the peptides may also include combinations thereof. These nanofibers and supramolecular hydrogels that contain combinations of enzymatically modified forms of the peptides may or may not contain a tethered therapeutic agent (Z).

Based on the various combinations of therapeutic agents, both tethered and micelle-containing, and combinations thereof, the peptides can be used to deliver the therapeutic agents in patients for the treatment of various disease conditions.

Therefore, one aspect of the invention relates to a method of delivering a therapeutic agent into the Golgi apparatus comprising encapsulating a therapeutic agent within a micelle structure of a pharmaceutical composition as described herein, and then contacting a cell with the pharmaceutical composition, whereby micelle structures are taken up by the cell and targeted to Golgi apparatus within the cell. As discussed hereinafter, the peptides of the invention form micelles, which turn into nanofibers after dephosphorylation or de-esterification. The formation of micelles of pS1 or AcS1 likely facilitates the cellular uptake by caveolin-mediated endocytosis, similar to the cellular uptake of peptide amphiphiles. In any event, when the micelle structure is altered by way of dephosphorylation or de-esterification of the component peptides, the micelle-delivered therapeutic agent is released into or adjacent the contacted cell.

Some embodiments of the method relate to the delivering of a therapeutic agent into Golgi apparatus wherein the cell is ex vivo or in vivo. For in vivo contact to occur, a pharmaceutical composition of the invention is administered to an individual administering is carried out orally, parenterally, subcutaneously, intravenously, intradermally, intramuscularly, intraperitoneally, by implantation, by intracavitary or intravesical instillation, intraarterially, intralesionally, intradermally, peritumorally, intratumorally, or by introduction into one or more lymph nodes. Other modes of administration that are effective to present the micelle to cells that are intentionally targeted (i.e., for delivery of the therapeutic agent) can also be used.

Administration of the pharmaceutical composition can be repeated on a daily schedule (i.e., once, twice, or thrice daily), or according to a periodic schedule (i.e., once weekly, bimonthly, once monthly).

Individuals that can be treated include both veterinary patients, typically but not exclusively mammals, as well as human patients.

A further aspect of the invention relates to a method of treating a patient having a cancerous condition. This method includes administering a pharmaceutical composition as described herein to a patient having a cancerous condition, where the administering is effective to inhibit cancer cell survival. Modes and frequency of administration, and patient groups include those identified above.

The cancerous conditions to be treated in accordance with this aspect can involve cancer cells present in a solid tumor, present as a metastatic cell, or present in a heterogenous population of cells that includes both cancerous and noncancerous cells. Exemplary cancer conditions include, without limitation, cancers or neoplastic disorders of the brain and CNS (glioma, malignant glioma, glioblastoma, astrocytoma, multiforme astrocytic gliomas, medulloblastoma, craniopharyngioma, ependymoma, pinealoma, hemangioblastoma, acoustic neuroma, oligodendroglioma, meningioma), pituitary gland, breast (Infiltrating, Pre-invasive, inflammatory cancers, Paget's Disease, Metastatic and Recurrent Breast Cancer), blood (Hodgkin's Disease, Leukemia, Multiple Myeloma, Lymphoma), lymph node cancer, lung (Adenocarcinoma, Oat Cell, Non-small Cell, Small Cell, Squamous Cell, Mesothelioma), skin (melanoma, basal cell, squamous cell, Kaposi's Sarcoma), bone cancer (Ewing's Sarcoma, Osteosarcoma, Chondrosarcoma), head and neck (laryngeal, pharyngeal, and esophageal cancers), oral (jaw, salivary gland, throat, thyroid, tongue, and tonsil cancers), eye, gynecological (Cervical, Endrometrial, Fallopian, Ovarian, Uterine, Vaginal, and Vulvar), genitourinary (Adrenal, bladder, kidney, penile, prostate, testicular, and urinary cancers), and gastrointestinal (appendix, bile duct (extrahepatic bile duct), colon, gallbladder, gastric, intestinal, liver, pancreatic, rectal, and stomach cancers).

In this aspect of the invention, the pharmaceutical composition contains the peptide in combination with a cancer therapeutic agent of the type described above, where the cancer therapeutic agent is tethered to the peptide chain, introduced separately to the pharmaceutical composition and encapsulated within the micelle structure, both are used together to deliver different forms of the same therapeutic agent (i.e., tethered and untethered forms of the same active agent), or both are used to deliver two different therapeutic agents in combination (i.e., one therapeutic agent tethered and the other untethered but encapsulated within the micelle structure).

While any class of antineoplastic agent, anticancer drug, or chemotherapeutic drug is contemplated for use in connection with the present invention, exemplary agents within these classes include alkylating agents, platinum drugs, antimetabolites, anthracycline and nonanthracycline antitumor antibiotics, topoisomerase inhibitors, mitotic inhibitors, corticosteroids and targeted cancer therapies (such as imatinib, Gleevec®; gefitinib, Iressa®; sunitinib, Sutent®; and bortezomib, Velcade®).

Yet another aspect of the invention relates to a method of treating a patient having Alzheimer's or Parkinson's disease. This method includes administering a pharmaceutical composition as described herein to a patient having a Alzheimer's or Parkinson's disease, wherein the administering is effective to treat symptoms of such disease. Modes and frequency of administration, and patient groups include those identified above.

In this aspect of the invention, the pharmaceutical composition contains the peptide in combination with a therapeutic agent suitable for treating Alzheimer's or Parkinson's disease, including those described above, where the therapeutic agent is tethered to the peptide chain, introduced separately to the pharmaceutical composition and encapsulated within the micelle structure, both are used together to deliver different forms of the same therapeutic agent (i.e., tethered and untethered forms of the same active agent), or both are used to deliver two different therapeutic agents in combination (i.e., one therapeutic agent tethered and the other untethered but encapsulated within the micelle structure).

Yet another aspect of the invention relates to a method of imaging a cell. The method includes providing a peptide or a composition as described herein, wherein Z₁ or Z₂ is a fluorophore, and then contacting a cell with the peptide or the composition, whereby the peptide, or micelle structures formed by the peptide, are taken up by the cell and targeted to the Golgi apparatus within the cell. Thereafter, an image of the cell can be obtained using any desired image capture equipment or techniques compatible with the fluorophore used as Z₁ and/or Z₂. In the image, the Golgi apparatus is identified by fluorescence from the fluorophore.

### EXAMPLES

The examples below are intended to exemplify the practice of embodiments of the disclosure but are by no means intended to limit the scope thereof.

### Materials and Methods

Reagents and Instruments: 2-Cl-trityl chloride resin (1.0 mmol/g), Fmoc protected amino acid, and HBTU were obtained from GL Biochem (Shanghai, China). N,N-diisopropylethylamine (DIEA) and solvents were obtained from Fisher Scientific. Alkaline phosphatase (“ALP”) was purchased from Biomatik (Cat. No. A1130, alkaline phosphatase [ALP], >1300 U/mg, in 50% glycerol.). Cysteamine S-phosphate, cysteamine hydrochloride and O-phosphorylethanolamine were all purchased from Sigma-Aldrich. All the chemical reagents and solvents were used as received from commercial sources without further purification. Minimum Essential Media (MEM), Dulbecco's Modified Eagle Medium (DMEM), McCoy's 5A Medium, and RPMI-1640 Medium were purchased from ATCC. Fetal bovine serum (FBS) and Penicillin-Streptomycin from Gibco by Life Technologies. All precursors and compounds were purified by a reverse phase HPLC (Agilent 1100 Series) equipped with an XTerra C18 RP column, and HPLC grade acetonitrile (0.1% TFA) and HPLC grade water (0.1% TFA) were used as the eluents. The LC-MS spectra were obtained with a Waters Acquity Ultra Performance LC with Waters MICROMASS detector. Transmission electron microscope (TEM) images were obtained on Morgagni 268 transmission electron microscope. 1H-NMR spectra of compounds were obtained using Varian Unity Inova 400 MHz. Fluorescence images were taken by ZEISS LSM 880 confocal laser scanning microscope.

Cell Lines: All cell lines used (HeLa, Saos-2, SJSA-1, OVSAHO, HCC1937, HepG2, OVCAR-4, SKOV-3, MCF-7, HEK293, HS-5) were purchased from the American Type Culture Collection (ATCC, Manassas, VA, USA). HeLa, HepG2, MCF-7 and HEK293 cells were cultured in MEM medium with 10% FBS and 1% P/S (100 U mL⁻¹ penicillin and 100 μg mL⁻¹ streptomycin, Invitrogen Life Technologies). Saos-2, SKOV-3 cells were cultured in McCoy's 5A medium with 15% FBS and 1% P/S. HS-5 cells were cultured in DMEM medium with 10% FBS and 1% P/S. SJSA-1, OVSAHO, HCC1937, OVCAR-4 cells were cultured in RPMI-1640 medium with 10% FBS and 1% P/S. All the cells were cultured at 37° C. in a humidified atmosphere of 5% CO₂. To determine the cytotoxicity of the compounds, cells were seeded in 96-well cell plate at 1.0×10⁴ cells/well for 24 h followed by culture medium removal, and then fresh culture medium containing compounds were added. For fluorescence imaging, cells were seeded in confocal dish at 1.5×10⁵ cells/dish for 24 h followed by the addition of fresh medium containing compounds, and then the fluorescence of cells were taken by ZEISS LSM 880 confocal laser scanning microscope with the 63× oil lens.

Critical micelle concentration (CMC) determination: A series of solutions of compounds (pS1, pS2), from the concentration of 0.0625 μM to 400 μM, were prepared in deionized water and adjusted the pH to 7.4. The count rates of the solutions were measured and recorded by an ALV/DLS/SLS-5000 Light Scattering System, which were converted to the intensity of scattered light and then plotted against concentrations to determine the CMC values. Then, ALP (0.1 U/mL) was added to the series of solutions and after 24 h, the same procedures were proceeded to determine CMC values of dephosphorylated pS1 and pS2 (S1 and S2).

Transmission Electron Microscopy Experiments: The 400 mesh copper grids coated with carbon film was glowing discharged and sample solutions (5 μL of each) was placed onto the grids. After 30 seconds, the sample solution was removed, and the grids were stained by uranyl acetate (2% v/v) and allowed to dry in air. TEM images were obtained with Morgagni 268 transmission electron microscope at the HV of 80 kV with filament of 2.

Determination of Dephosphorylation Rates in vitro: To determine the dephosphorylation rates of thiophosphopeptides in vitro, pS1, pO1, pS2, pO2 solutions (PBS, pH 7.4) were prepared at concentration of 120 μM and ALP was added into the solutions to make the final concentration of ALP to 0.1 U/mL at 37° C. At the designated time, the same volume of methanol was added to eliminate the activity of ALP. LC/MS was used analyse the results.

Staining cells with FlAsH-EDT 2: Briefly, after incubating HeLa cell lines (1.5×10⁵) in 3.5 cm confocal dish for 24 h at 37° C. in a humidified atmosphere of 5% CO₂, we discarded the medium and added fresh medium containing pS2 (10 μM) and incubated them together for 4 h. After 4 h, medium was removed followed by three-time wash by cell imaging buffer to eliminate FBS. Then, we added FlAsH-EDT₂ solution (cell imaging buffer, 1 μM) into the confocal dish and incubated the HeLa cells at 37° C. for 1 h. Meanwhile, we prepared 1,2-Ethanedithiol (EDT) solution (cell imaging buffer, 250 μM) and after the one-hour incubation of HeLa cells with FlAsH-EDT₂, FlAsH-EDT₂ was discarded and EDT solution was added to the confocal dish to eliminate the unspecific binding of FlAsH-EDT₂ for 10 min. Ten minutes later, EDT solution was removed and cells were washed with cell imaging buffer three times and the fluorescence images were taken by ZEISS LSM 880 confocal laser scanning microscope.

### Example 1—Synthesis of Peptides and Precursors

The precursor NBD-β-Alanine was synthesized according to Scheme 1 below:

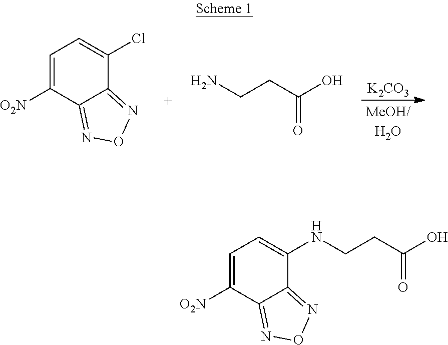

Briefly, to a 10 mL water solution of (3-Alanine (5.5 mmol, 490 mg) and potassium carbonate (16.5 mmol, 2.07g), NBD-Cl (5 mmol, 1g) in 60 mL of MeOH was added to the above solution, dropwise with stirring under nitrogen gas protection. After stirring at room temperature for 6 h, methanol was removed by a rotary evaporator and the residual solution was acidified to pH 3 by HCl (1 N). The acidic aqueous solution was then extracted by diethyl ether. The combined organic solution was dried over anhydrous sodium sulfate, and then concentrated by a rotary evaporator. The resulting dark-yellow powder (NBD-β-Alanine) was directly used for solid phase peptide synthesis.

The synthesis of all peptides was performed by solid phase synthesis. Standard Fmoc chemistry solid phase peptide synthesis was carried out using 2-chlorotrityl chloride resin and the corresponding Fmoc-protected amino acids with side chains properly protected. Briefly, the 2-Cl resin (1 g) was swelling in dry DCM for 30 min and then the first amino acid was loaded onto the resin. After loading the first amino acid to the resin, the capping regent (DCM:MeOH:DIEA=17:2:1) was used to cap all the active sites of the resin for additional 15 min. Fmoc group was then removed with 20% piperidine in DMF, the next Fmoc-protected amino acid was coupled to the free amino group using HBTU as the coupling reagent. The peptide chain was cleaved from the resin by 95% TFA (95% TFA, 2.5% TIPS, 2.5% H₂O) for 1 h. After the solvent was removed by a rotary evaporator, 30 mL of dry diethyl ether was added to the residual solution and followed by centrifuging at 10000 rpm for 8 min. The resulting solid products were dried by a lyophilizer. NBD-containing peptides were obtained as a solid orange powder and naphthylalanine-capped peptides were obtained as solid white powder.

Synthesis of pS1 and pS2: Briefly, 0.1 mmol of synthesized peptide (NBD-ff or Nap-ff) was dissolved in 2 mL dry DMF and 0.12 mmol of HBTU was added directly into the solution. After stirring the mixture for 30 minutes, 0.12 mmol of Cysteamine S-Phosphate sodium salt was added into the mixture and DIEA was added dropwise to adjust pH around 8, then keep stirring for 8 h. After that, the solvent is air dried and the remained oily product was dissolved in methanol and purified by RP-HPLC to obtain NBD-ff and Nap-ff bearing the C-terminal aminoethyl-thiophsphate group.

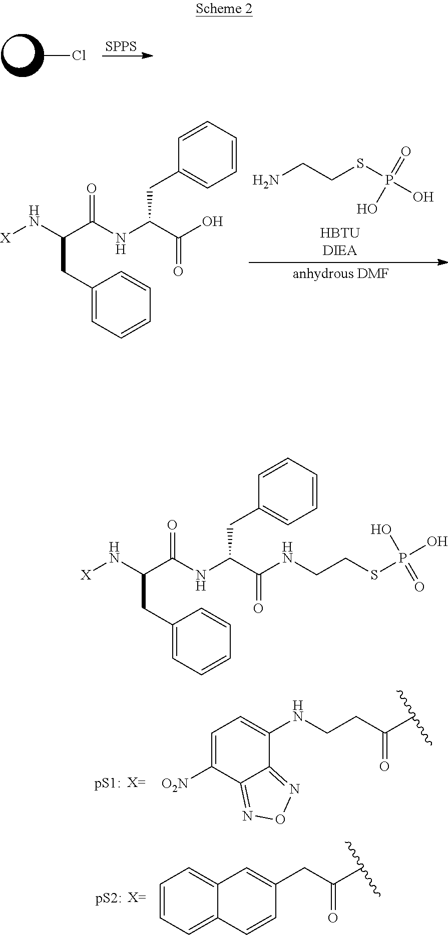

LC/MS data was obtained for pS1 and pS2 to confirm their structure. The calculated molecular weight (Mw) of pS1 is 685.17, and the observed M/Z=684.32. The calculated molecular weight (Mw) of pS2 is 619.19, and the observed M/Z=618.45.

Synthesis of SI: Briefly, 0.1 mmol of synthesized peptide (NBD-ff) was dissolved in 2 mL dry DMF and 0.12 mmol of HBTU was added directly into the solution. After stirring the mixture for 30 minutes, 0.12 mmol of cysteamine hydrochloride was added into the mixture and DIEA was added dropwise to adjust pH around 8, and stirring was continued for 8 h. After that, the solvent was air dried and the remaining oily product was dissolved in methanol and purified by RP-HPLC to obtain the titled compound.

Synthesis of pO1 and pO2: Briefly, 0.1 mmol of synthesized peptide (NBD-ff or Nap-ff) was dissolved in 2 mL dry DMF and 0.12 mmol of HBTU was added directly into the solution. After stirring the mixture for 30 minutes, 0.12 mmol of O-Phosphorylethanolamine was added into the mixture and DIEA was added dropwise to adjust pH around 8, and stirring was continued for 8 h. After that, the solvent was air dried and the remaining oily product was dissolved in methanol and purified by RP-HPLC to obtain the titled compounds. LC/MS data was obtained for pO1 and pO2 to confirm their structure. The calculated molecular weight (Mw) of pO1 is 669.19, and the observed M/Z=668.46. The calculated molecular weight (Mw) of pO2 is 603.21, and the observed M/Z=602.46.

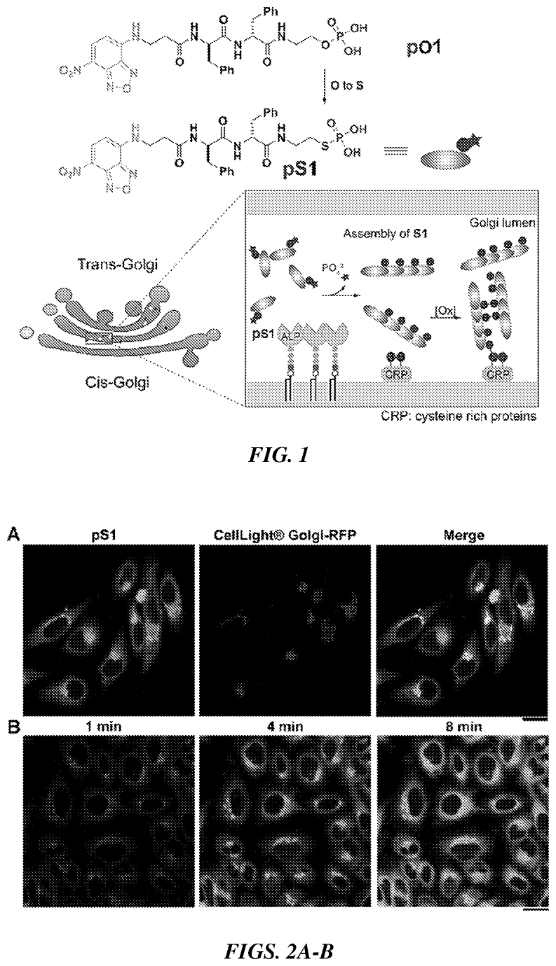

As shown in FIG. 1, pS1 contains three functional segments: (i) 4-nitro-2,1,3-benzoxadiazole (NBD), a fluorophore that emits bright green fluorescence in hydrophobic environment of supramolecular assemblies (Gao et al., “Imaging Enzyme-triggered Self-assembly of Small Molecules Inside Live Cells,” Nat. Commun. 3:1-8 (2012), which is hereby incorporated by reference in its entirety); (ii) D-diphenylalanine (ff), a hydrophobic building block, which enables self-assembly and resists proteolysis; (iii) thiophosphate group (present in the C-terminal aminoethyl-thiophosphate group). The thiophosphate group is a substrate of ALP (Herrington et al., “Studies on Latent Derivatives of Aminoethanethiols as Potentially Selective Cytoprotectants IV. Enzymatic Hydrolysis of Cysteamine-S-phosphate,” Cancer Res. 27:148-151 (1967), which is hereby incorporated by reference in its entirety), and its removal promotes enzymatic self-assembly (Yang et al., “Enzymatic Formation of Supramolecular Hydrogels,” Adv. Mater. 16:1440-1444 (2004); Zhou et al., “Taurine Boosts Cellular Uptake of Small D-peptides for Enzyme-instructed Intracellular Molecular Self-assembly,” J. Am. Chem. Soc. 137:10040-10043 (2015), which are hereby incorporated by reference in their entirety). The NBD fluorophore is linked to the ff dipeptide via β-Ala residue. Solid phase peptide synthesis (SPPS) of NBD-ff followed by a conjugation of cysteamine S-phosphate (Akerfeldt, S., “Hydrolysis of Cysteamine S-Phosphate,” J. Org. Chem. 29:493 (1964), which is hereby incorporated by reference in its entirety) generates pS1 (Scheme 2) in a good yield.

Based on the results presented herein, it is contemplated that other peptide cores that enable self-assembly and resist proteolysis can be used in replacement of the di-D-phenylalanine core.

### Example 2—Evaluation of Peptide Properties

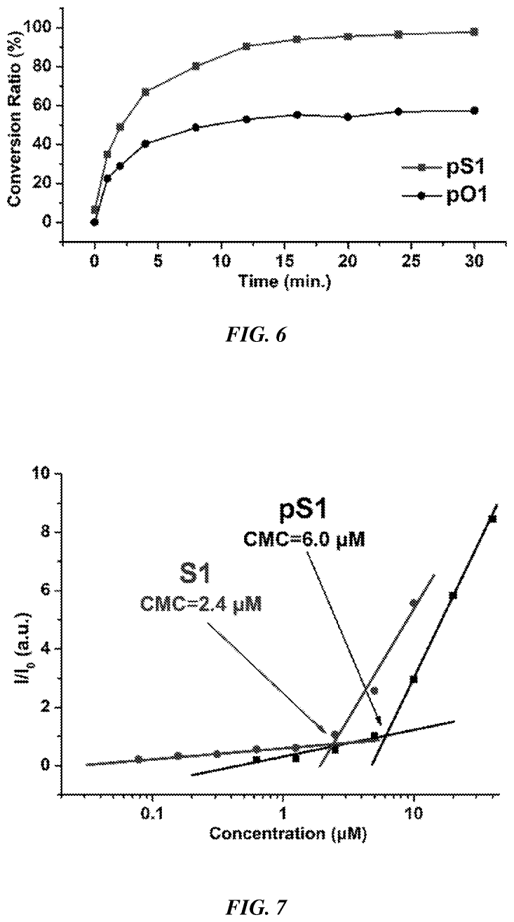

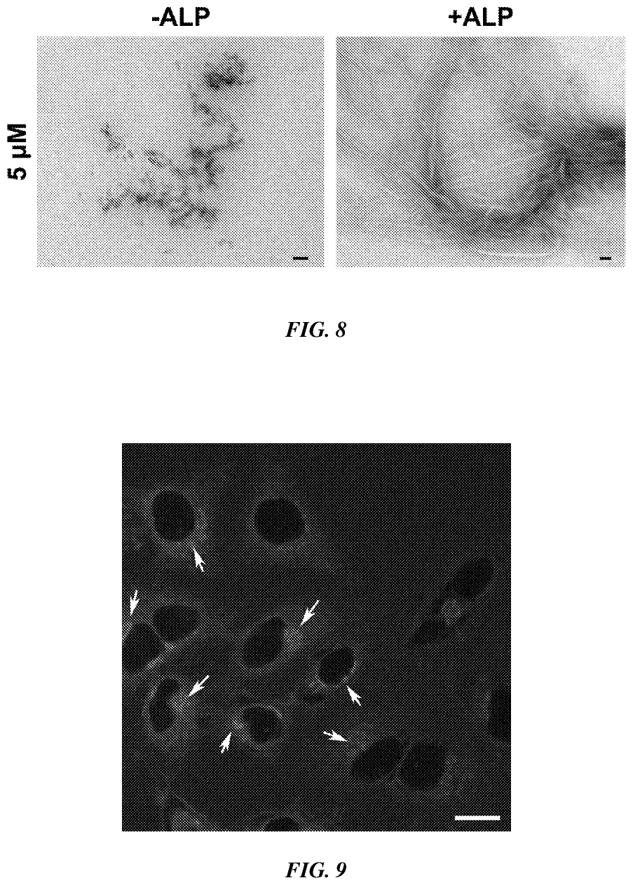

The design of pS1 ensures the fast dephosphorylation of the thiophosphopeptide by ALP (FIG. 6). While pS1 exhibits critical micelle concentration (CMC) of 6.0 μM, S1 has a CMC of 2.4 μM (FIG. 7). Transmission electron microscopy (TEM) reveals that pS1, at 5 μM forms micelles, which turn into nanofibers after ALP converts pS1 to S1 (FIG. 8). The formation of micelles of pS1 likely facilitates the cellular uptake by caveolin-mediated endocytosis, similar to the cellular uptake of peptide amphiphiles (Liang et al., “Enhanced Cellular Uptake and Nuclear Accumulation of Drug-peptide Nanomedicines Prepared by Enzyme-instructed Self-assembly,” J. Control. Release. 317:109-117 (2020); Lock et al., “Tuning Cellular Uptake of Molecular Probes by Rational Design of Their Assembly into Supramolecular Nanoprobes,” J. Am. Chem. Soc. 138:3533-3540 (2016); Wang et al., “Crescent-Shaped Supramolecular Tetrapeptide Nanostructures,” J. Am. Chem. Soc. 142:20058-20065 (2020); Feng et al., “Enzymatic Assemblies).

### Example 3—Cell Uptake and Golgi Apparatus Targeting

HeLa cells were incubated with CellLight® Golgi-RFP (Herrera et al., “A Bispecific Antibody Promotes Aggregation of Ricin Toxin on Cell Surfaces and Alters Dynamics of Toxin Internalization and Trafficking,” PLoS One 11:(6):e0156893 (2016), which is hereby incorporated by reference in its entirety) for 24 hours to transfect RFP at the Golgi, and then the HeLa cells were incubated with pS1 (10 μM) for 8 minutes (FIG. 2A-B). The fluorescence from the assemblies of S1 overlaps with the red fluorescence from all Golgi-RFP, confirming that pS1 targets the Golgi of the HeLa cells. The fluorescence of S1 appears almost instantly after adding pS1 in the culture of HeLa cells (FIG. 9). This rate is at least an order of magnitude faster than previously reported probes (van Echten-Deckert, et al., “1-Methylthiodihydroceramide, A Novel Analog of Dihydroceramide, Stimulates Sphinganine Degradation Resulting in Decreased De Novo Sphingolipid Biosynthesis,” J. Biol. Chem. 273:1184-91 (1998); Zhang et al., “An Off-on COX-2-specific Fluorescent Probe: Targeting the Golgi Apparatus of Cancer Cells,” J. Am. Chem. Soc. 135″11663-11669 (2013); Li et al., “Chiral Nanoprobes for Targeting and Long-term Imaging of the Golgi Apparatus. Chem. Sci. 8:6829-6835 (2017), which are hereby incorporated by reference in their entirety). The intensity of the fluorescence at the Golgi increases significantly with the time of incubation of pS1, about 7 times enhancement from 1 minute to 8 minutes. Except the bright fluorescence at the Golgi and the dim fluorescence at the endoplasmic reticulum (ER), the rest of intracellular and extracellular regions of the HeLa remain dark. This result indicates that enzymatic assembly of Si occurs at the Golgi of HeLa cells, agreeing with the observation of ALP at Golgi of HeLa cells (Sasaki and Fishman, “Ultrastructural Studies on Regan and Non-Regan Isoenzymes of Alkaline Phosphatase in Human Ovarian Cancer Cells,” Cancer Res. 33:3008-3018 (1973), which is hereby incorporated by reference in its entirety). Live cell imaging over 20 minutes revealed that the fluorescence of the assemblies of S1 emerges at the Golgi prior to diffusing to ER, likely resulting from Golgi-ER transport (Lee et al., “Bi-directional Protein Transport Between the ER and Golgi,” Annu. Rev. Cell. Dev. Biol. 20:87-123 (2004), which is hereby incorporated by reference in its entirety). The differential interference contrast (DIC) image of HeLa cells treated with 10 μM of pS1 for 20 min demonstrated well-spread HeLa cells, excluding the possibility that pS1 enters cells due to cell death.

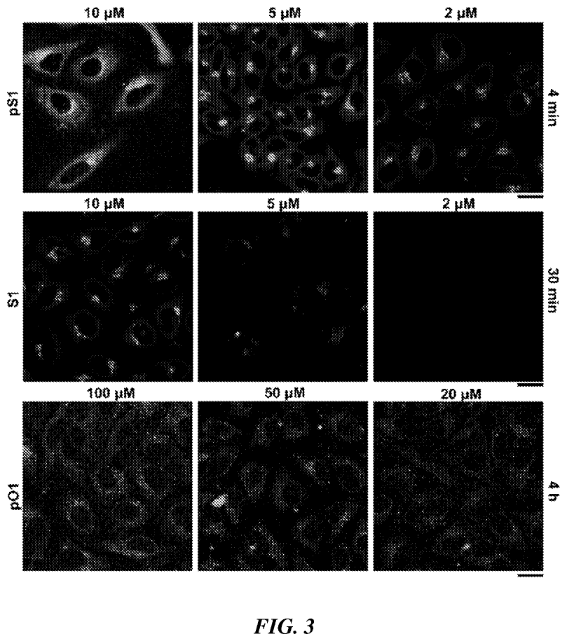

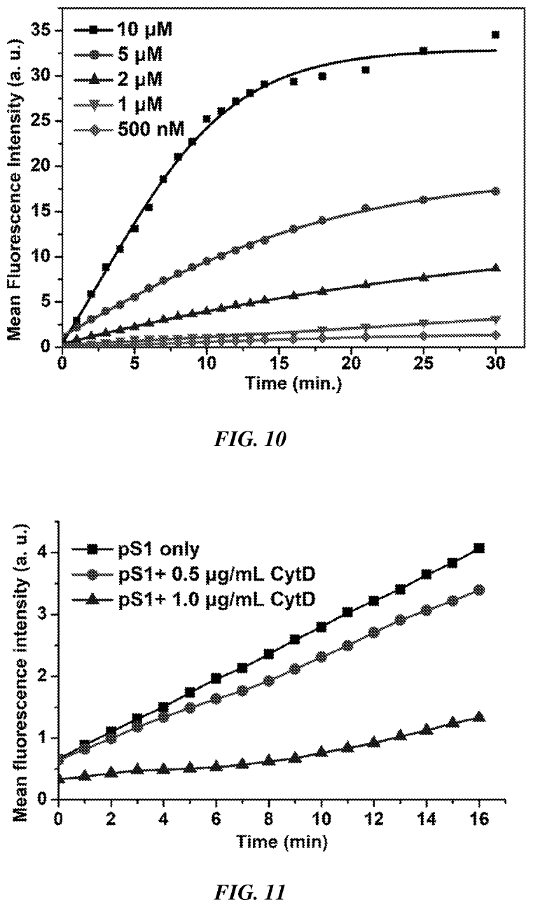

The concentration of pS1 is another important factor that determines the accumulation of S1 at Golgi. Golgi fluorescence was assessed by fixing the incubation time at 4 minutes and varying the concentration pS1 at 10, 5, and 2 μM (FIG. 3). At 10 μM, bright green fluorescence presents in Golgi and weak fluorescence in ER; at 5 μM, green fluorescence clearly still presents at the Golgi, but little at the ER; and at 2 μM, much weaker fluorescence at the Golgi. Decreasing the concentration of pS1 to 500 nM still results in Golgi accumulation of S1 in HeLa cells, although the brightness of S1 at Golgi is weaker at the beginning of the addition and distinctive fluorescence appears at the Golgi after 15 minutes. The accumulation rate of S1 at Golgi, quantified by the increase of the fluorescence intensity, is concentration dependent (FIG. 10). Use of a Golgi disruptor, brefeldin A (BFA), abolishes the accumulation of Si. These results indicate that enzymatic formation and self-assembly of S1 in situ at Golgi enables the instant targeting of Golgi. In addition, the concentration needed for pS1 targeting of Golgi is much less than the previously reported probes (van Echten-Deckert, et al., “1-Methylthiodihydroceramide, A Novel Analog of Dihydroceramide, Stimulates Sphinganine Degradation Resulting in Decreased De Novo Sphingolipid Biosynthesis,” J. Biol. Chem. 273:1184-91 (1998); Zhang et al., “An Off-on COX-2-specific Fluorescent Probe: Targeting the Golgi Apparatus of Cancer Cells,” J. Am. Chem. Soc. 135″11663-11669 (2013); Li et al., “Chiral Nanoprobes for Targeting and Long-term Imaging of the Golgi Apparatus. Chem. Sci. 8:6829-6835 (2017), which are hereby incorporated by reference in their entirety).

Unlike pS1, S1, at the concentration of 10 μM and when incubated with HeLa cells for 8 minutes, hardly results in any fluorescence in the cells, indicating slower cell uptake of S1 than that of pS1 and indicating the importance of enzymatic dephosphorylation for targeting Golgi. As shown in FIG. 3, after incubating S1 (10 μM) with HeLa cells for 30 minutes, some green fluorescence was observed at the Golgi of the HeLa cells, with the fluorescent intensity similar to that of HeLa cells incubated with pS1 (2 μM) for 4 minutes. After 30 minutes incubation, when the concentrations of S1 are at 5 and 2 μM, there is very weak fluorescence at the Golgi and no fluorescence in cell at all, respectively. These results indicate that S1 enters the HeLa cells less efficiently than pS1. Moreover, pO1 (the parent compound of pS1) produces almost no fluorescence in the HeLa cells after 8 minutes incubation and at the concentration of 10 μM. In fact, after 4 hours of incubation of pO1 and HeLa cells, there are several scattered fluorescent puncta in cells, with fluorescent intensity being proportional to the concentrations of pO1 (from 100 to 50 and to 20 μM). These results indicate that the assemblies of O1, formed by dephosphorylation, largely are retained in endosomes or lysosomes (FIG. 3). The above results confirm the unique ability of pS1 for rapid targeting of the Golgi.

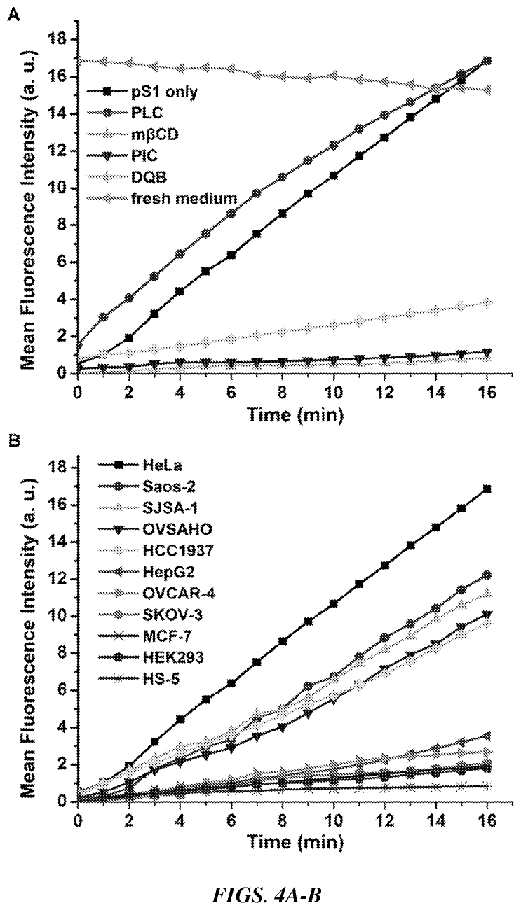

To further understand the mechanism of Golgi-accumulation of S1 assemblies resulting from the rapid enzymatic dephosphorylation of pS1, the rate of fluorescence increase in Golgi was examined over a 16 minute period in HeLa cells treated pS1 and several inhibitors. The fluorescence increase in the Golgi of HeLa cells incubated with pS1 was the only reference (FIG. 4A). Using phospholipase C (PLC) (Low and Finean, “Release of Alkaline Phosphatase From Membranes by a Phosphatidylinositol-specific Phospholipase C,” Biochem. J. 167:281-284 (1977), which is hereby incorporated by reference in its entirety), an enzyme that cleaves glycosylphosphatidylinositol (GPI) anchor, to remove ALP from the cell membrane results in slightly faster increase of fluorescence in the Golgi, confirming that ALP at Golgi dephosphorylates pS1 and indicating that pericellular dephosphorylation of pS1 by the ALP on plasma membrane slightly slows the accumulation of Si at the Golgi. Methyl-β-cyclodextrin (mβCD) significantly decreases the rate of the fluorescence increase at the Golgi, indicating that pS1 (at 10 μM) likely enters cells via caveolin-mediated endocytosis. As a potent inhibitor of actin polymerization (Gottlieb et al., “Actin Microfilaments Play a Critical Role in Endocytosis at the Apical But Not the Basolateral Surface of Polarized Epithelial Cells,” J Cell Biol 120:695-710 (1993), which is hereby incorporated by reference in its entirety), cytochalasin D (CytD) decreases the accumulation of S1 at Golgi in a concentration-dependent manner (FIG. 11). This result agrees with the critical role of actin dynamics in cellular uptake, indicating that pS1 also enters the cells via marcopinocytosis (Nakase et al., “Cellular Uptake of Arginine-rich Peptides: Roles for Macropinocytosis and Actin Rearrangement,” Mol. Ther. 10:1011-1022 (2004), which is hereby incorporated by reference in its entirety). Both the phosphatase inhibitor cocktail set 3 (PIC) and the tissue nonspecific ALP inhibitor DQB (Dahl et al., “Discovery and Validation of a Series of Aryl Sulfonamides as Selective Inhibitors of Tissue-Nonspecific Alkaline Phosphatase (TNAP),” J. Med. Chem. 52:6919-6925 (2009), which is hereby incorporated by reference in its entirety)) reduce the rate of fluorescence increase at the Golgi, with PIC more effectively inhibiting the accumulation than DQB. These results indicate that other phosphatases, in addition to ALP, also contribute to the enzymatic accumulation of S1 at Golgi and agree with sorting of ALP at the Golgi before secretion (Goldfischer, “The Internal Reticular Apparatus of Camillo Golgi: A Complex, Heterogeneous Organelle, Enriched in Acid, Neutral, and Alkaline Phosphatases, and Involved in Glycosylation, Secretion, Membrane Flow, Lysosome Formation, and Intracellular Digestion,” J. Histochem. Cytochem 30:717-33 (1982); Paladino et al., “Golgi Sorting Regulates Organization and Activity of GPI Proteins at Apical Membranes,” Nat. Chem. Biol. 10:350-357 (2014), which are hereby incorporated by reference in their entirety).

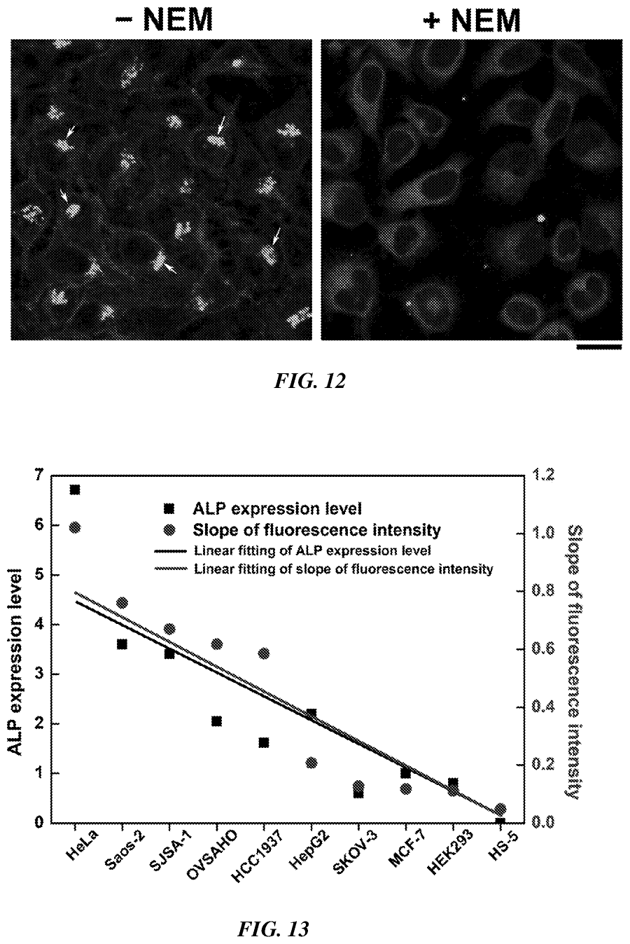

The exocytosis of accumulated S1 was also examined by incubating HeLa cells pretreated with pS1 in a fresh culture medium. The intensity of fluorescence at the Golgi of the HeLa cells drops only slightly over time, confirming that the enzymatically formed assemblies of Si are largely trapped in the Golgi. Inhibiting protein disulfide isomerases (PDIs) decrease disulfide bonds of cysteine rich proteins (CRPs) (Lyles and Gilbert, “Catalysis of the Oxidative Folding of Ribonuclease A By Protein Disulfide Isomerase: Pre-steady-state Kinetics and the Utilization of the Oxidizing Equivalents of the Isomerase,” Biochemistry 30:619-625 (1991), which is hereby incorporated by reference in its entirety) that are transported to Golgi, contributing to a slight decrease of the rate of Golgi accumulation of Si. This result implies the peptide assemblies likely form disulfide bonds with CRPs. This observation agrees with the formation of dimers of S1 (or S2) in the cell lysate treated with pS1 (or pS2), suggesting that a certain extent of covalent linkage between assemblies likely contributes to the retention of the assemblies in the Golgi. Moreover, the addition of N-ethylmaleimide almost completely eliminates the accumulation of S1 at Golgi (FIG. 12), further supporting the belief that S1 forms disulfide bond with CRPs at Golgi.

### Example 4—Golgi Targeting in Multiple Cancer Cell Lines

To examine the applicability of the process illustrated in FIG. 1 for targeting Golgi of other cells, we incubated pS1 with several other cancer cell lines (Saos-2, SJSA-1, OVSAHO, HCC1937, HepG2, OVCAR-4, SKOV-3, MCF-7) and immortalized normal cell lines (HEK293 and HS-5) and examined the rates of fluorescent increase at the Golgi of the cells (FIG. 4B). The fluorescence intensities increase significantly at the Golgi of Saos-2, SJSA-1, OVSAHO and HCC1937 cells, slightly in those of HepG2 and OVCAR4 cells, and much slowly in those of SKOV3, MCF7, HEK293 and HS-5 cells. These results largely agree with expression levels of ALP in these cell (FIG. 13). One exception is HepG2, which expresses higher level of ALP than OVSAHO, but exhibits slower fluorescence increase at Golgi. High level of glutathione in hepatocytes (Kretzschmar, M., “Regulation of Hepatic Glutathione Metabolism and its Role in Hepatotoxicity,” Exp. Toxicol. Pathol. 48:439-446 (1996), which is hereby incorporated by reference in its entirety) likely antagonizes the accumulation of S1 in the Golgi. This observation supports the understanding that oxidative Golgi environments favors disulfide formation and the retention of the assemblies of S1 at the Golgi.

### Example 5—Synthesis and Evaluation of Non-fluorescent Peptide Analogue

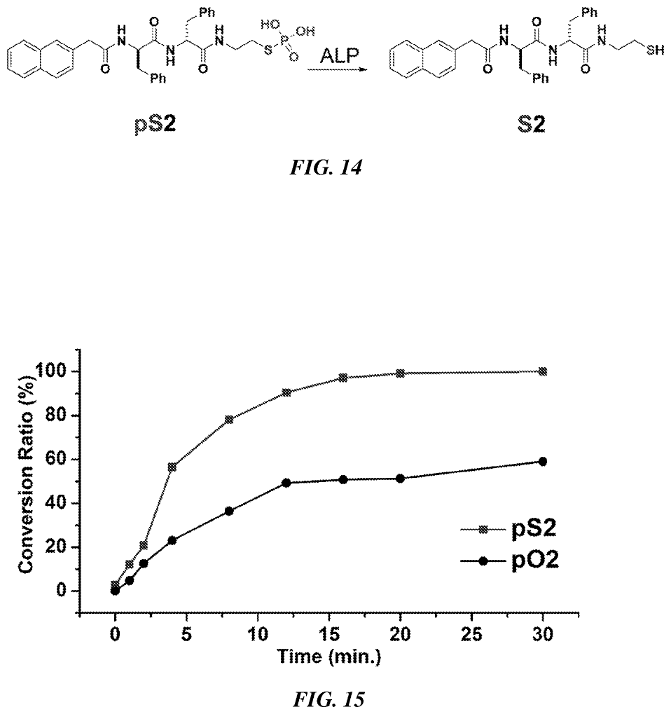

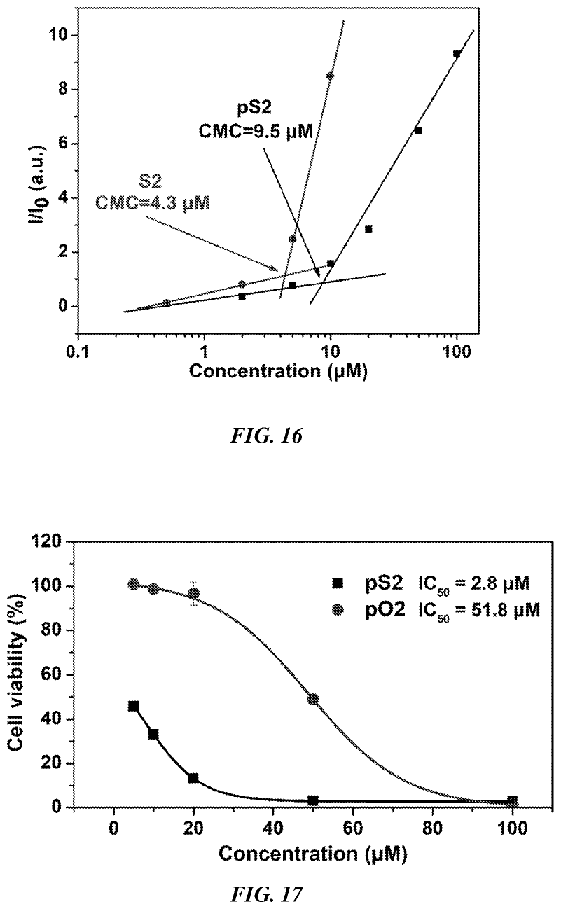

Using the same solid-phase synthesis described in Example 1, a nonfluorescent analogue of pS1 (pS2) was synthesized using naphthyl group to replace NBD (FIG. 14). Being similar to pS1, pS2 undergoes rapid dephosphorylation by ALP to form S2 (FIG. 14). Compared with its oxophosphate analogue pO2 (Feng et al., “Instructed-assembly of Small Peptides Inhibits Drug-Resistant Prostate Cancer Cells,” Pept. Sci. 112:e24123 (2020), which is hereby incorporated by reference in its entirety), pS2 shows much faster dephosphorylation. For example, when incubated with ALP (0.1 U/mL) for about 16 minutes, pS2 nearly fully converted to S2, while the maximum conversion ratio of pO2 to O2 remains at about 50% over the same duration (FIG. 15). The CMC of pS2 is 9.5 μM, and the CMC of its dephosphorylated product, S2, is 4.3 μM (FIG. 16), indicating both compounds have an excellent self-assembling ability.

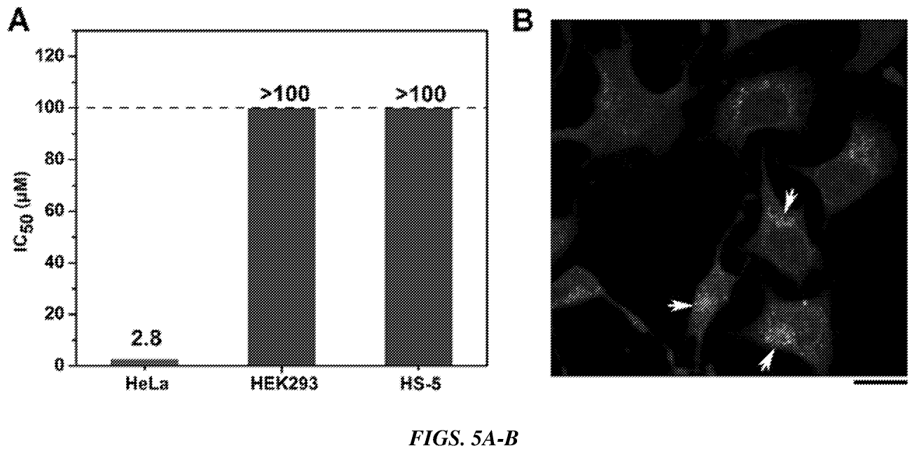

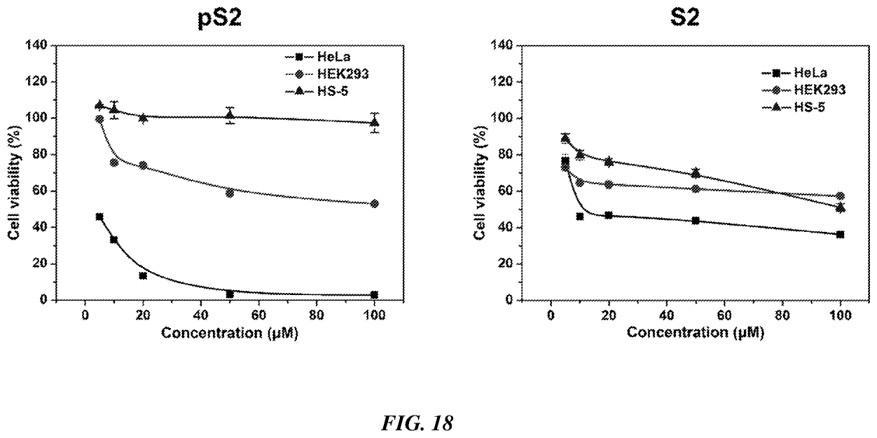

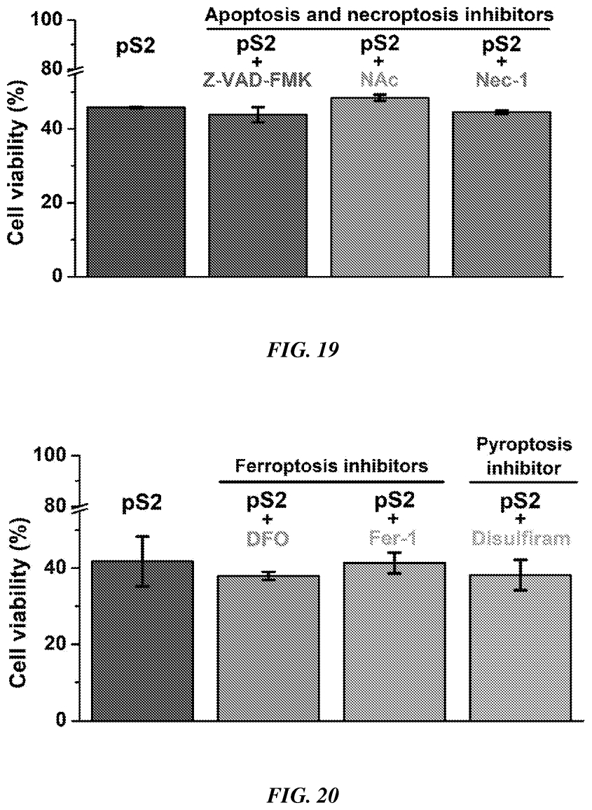

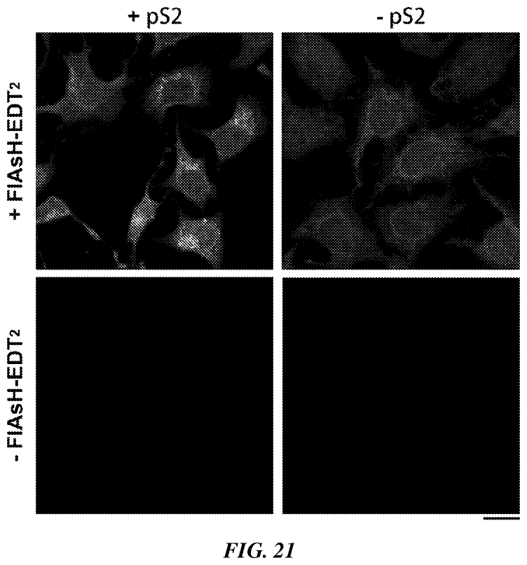

The cytotoxicity of pS2 was assessed against HeLa, HEK293, and HS-5 cells, and the IC50 values were 2.8 μM, >100 μM and >100 μM, respectively (FIG. 5A). The inhibitory activity of pS2 against HeLa cells is an order of magnitude higher than that of pO2 (FIG. 17). The difference between these IC₅₀ values agrees with the difference of the rate of enzymatic assemblies in Golgi of the cells, indicating that selectively targeting the Golgi is the result of fast enzyme kinetics. This result also confirms that pS2 is more selective than S2 against cancer cells (FIG. 18). In addition, several commonly used inhibitors (Z-VAD-FMK, NAc, Nec-1, DFO, Fer-1, and disulfiram) (Tang et al., “The molecular Machinery of Regulated Cell Death,” Cell Res. 29:347-364 (2019), which is hereby incorporated by reference in its entirety) of cell death were unable to rescue HeLa cells from pS2 (FIGS. 19 and 20), indicating that a unique cell death resulted from the molecular processes defined by thiophosphopeptides at the Golgi apparatus. Treating the HeLa cells incubated with pS2 by the tetracysteine probe, FlAsH-EDT₂, (Adams et al., “New Biarsenical Ligands and Tetracysteine Motifs for Protein Labeling in Vitro and in Vivo: Synthesis and Biological Applications,” J. Am. Chem. Soc. 124:6063-6076 (2002), which is hereby incorporated by reference in its entirety) results in the fluorescent at the Golgi of the HeLa cells (shown by the arrow in FIGS. 5B and 21), indicating that S2 self-assembles at the Golgi to arrange multiple C-terminal thiols in a manner similar to that of tetracysteine.

### Discussion of Examples 1-5

The preceding examples illustrate that rapid dephosphorylation of thiophosphopeptides enables instantly targeting of Golgi apparatus and selectively inhibiting cancer cell survival. These observations agree with several known facts:
(i) CRPs are enriched in Golgi (Aoki et al., “Golgi Retention of a Trans-Golgi Membrane Protein, Galactosyltransferase, Requires Cysteine and Histidine Residues Within the Membrane-anchoring Domain,” Proc. Natl. Acad. Sci. U.S.A. 89:4319-4323 (1992); Maeda et al., “Recruitment of Protein Kinase D to the Trans-Golgi Network Via the First Cysteine-rich Domain,” EMBO J. 20:5982-5990 (2001); Zhang et al., “Two-photon Fluorescence Imaging Reveals a Golgi Apparatus Superoxide Anion-mediated Hepatic Ischaemia-reperfusion Signalling Pathway,” Chem. Sci. 10:879-883 (2019), which are hereby incorporated by reference in their entirety);
(ii) a significant level of oxidation occurs in the Golgi membrane (Hatori et al., “Visualization of the Redox Status of Cytosolic Glutathione Using the Organelle-and Cytoskeleton-targeted Redox Sensors,” Antioxidants 9(2):129 (2020), which is hereby incorporated by reference in its entirety)
(iii) ALP, as an “almost perfect” enzyme (Simopoulos and Jencks, “Alkaline Phosphatase Is an Almost Perfect Enzyme,” Biochemistry 33:10375-10380 (1994), which is hereby incorporated by reference in its entirety) being anchored on the cell membrane by glycosylphosphatidylinositol (GPI) and overexpressed on certain cancer cell (Sasaki and Fishman, “Ultrastructural Studies on Regan and Non-Regan Isoenzymes of Alkaline Phosphatase in Human Ovarian Cancer Cells,” Cancer Res. 33:3008-3018 (1973); Fishman et al., “Immunology and Biochemistry of Regan Isoenzyme of Alkaline Phosphatase in Human Cancer,” Nature 219:697-699 (1968); Vijayan et al., “Targeting Immunosuppressive Adenosine in Cancer,” Nat. Rev. Cancer 17:709-724 (2017), which are hereby incorporated by reference in their entirety), is known to be sorted as oligomers at the Golgi before secretion (Goldfischer, “The Internal Reticular Apparatus of Camillo Golgi: A Complex, Heterogeneous Organelle, Enriched in Acid, Neutral, and Alkaline Phosphatases, and Involved in Glycosylation, Secretion, Membrane Flow, Lysosome Formation, and Intracellular Digestion,” J. Histochem. Cytochem 30:717-33 (1982); Paladino et al., “Golgi Sorting Regulates Organization and Activity of GPI Proteins at Apical Membranes,” Nat. Chem. Biol. 10:350-357 (2014), which are hereby incorporated by reference in their entirety).
   Since thiophosphopeptides (Parker et al., “Optimizing Thiophosphorylation in the Presence of Competing Phosphorylation With MALDI-TOF-MS Detection,” J. Proteome Res. 4:1863-1866 (2005); KÜNg and Bannwarth, “Chemical Synthesis of o-thiophosphotyrosyl Peptides,” Int. J. Pept. Protein Res. 43:146-153 (1994); Allard et al., “Synthesis of Phosphopeptides in the Fmoc Mode,” Int. J. Pept. Res. Ther. 13:447-468 (2007), which are hereby incorporated by reference in their entirety) are much less developed than phosphopeptides, replacinge NBD with other functional motifs (e.g. 10-hydroxycamptothecine) (Cai et al., “Supramolecular “Trojan Horse” for Nuclear Delivery of Dual Anticancer Drugs,” J. Am. Chem. Soc. 139:2876-2879 (2017), which is hereby incorporated by reference in its entirety) may lead to new discoveries. These thiophosphopeptides also may serve as the substrates for thiol-click chemistry (Liang et al., “A Biocompatible Condensation Reaction for Controlled Assembly of Nanostructures in Living Cells,” Nat. Chem. 2:54-60 (2010), which is hereby incorporated by reference in its entirety) or for integration thiol groups in other supramolecular assemblies (Shigemitsu et al., “Protein-responsive Protein Release of Supramolecular/Polymer Hydrogel Composite Integrating Enzyme Activation Systems,” Nat. Commun. 11:3859 (2020); Tanaka et al., “Cancer Cell Death Induced by the Intracellular Self-Assembly of an Enzyme-Responsive Supramolecular Gelator,” J. Am. Chem. Soc. 137:770-775 (2015); Pires et al., “Controlling Cancer Cell Fate Using Localized Biocatalytic Self-Assembly of an Aromatic Carbohydrate Amphiphile,” J. Am. Chem. Soc. 137:576-579 (2015); Merg et al., “2D Crystal Engineering of Nanosheets Assembled from Helical Peptide Building Blocks,” Angew. Chem. Int. Ed. 58:13507-13512 (2019); Liu et al., “Dual-Functionalized Crescent Microgels for Selectively Capturing and Killing Cancer Cells,” Angew. Chem. Int. Ed. 59:14076-14080 (2020), which are hereby incorporated by reference in their entirety).

### Example 6—Synthesis of Additional Peptides and Precursors

The fluorophore-labeled precursor peptide, NBD-ff, was synthesized according to Example 1. 0.1 mmol of synthesized peptide was dissolved in 2 mL dry DMF and 0.12 mmol of HBTU was added directly into the solution. After stirring the mixture for 30 minutes, 0.12 mmol of S-(2-aminoethyl) ethanethioate hydrobromide was added into the mixture and DIEA was added dropwise to adjust pH around 8, with stirring for an additional 8 h. After that, the solvent was air dried and the remaining oily product was dissolved in methanol and purified by RP-HPLC to obtain NBD-ff bearing the C-terminal aminoethyl-thioester group, designated AcS1. The same procedure will be carried out using the intermediate peptide, Nap-ff, to generate AcS2.

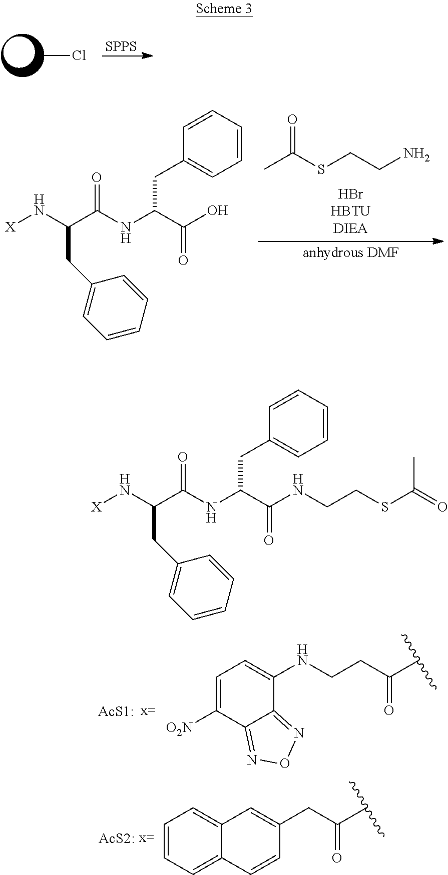

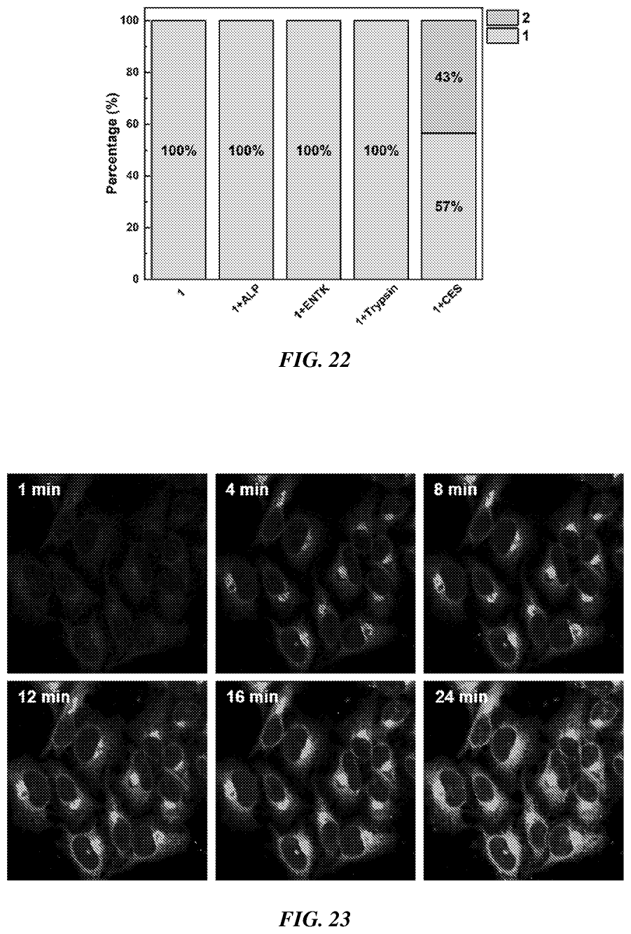

The responsiveness of AcS1 to various enzymes was next assessed in a cell free assay by treating AcS1 with alkaline phosphatase (ALP, 0.1 U/mL), enterokinase (ENTK, 1 U/mL), trypsin (1 U/mL), and carboxylesterase (CES, 1 U/mL) for 24 h. Measurement of AcS1 or its de-esterified product, 51, was performed by mass spectroscopy. The results are shown in FIG. 22, which indicate that AcS1 is responsive only to the carboxylesterase (43% converted).

### Example 7—Cell Uptake and Golgi Apparatus Targeting

HeLa cells were treated with AcS1 (10 μM) in accordance with the preceding Examples, and peptide uptake was measured over a period of time using CLSM. As shown in FIG. 23, AcS1 is taken up quickly, with detectable fluorescence by 4 min and increasing fluorescence until 24 min. This confirms AcS1 uptake and Golgi-accumulation of S1 assemblies resulting from the rapid enzymatic de-esterification of AcS1 in much the same manner shown for pS1.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

1. A peptide comprising the structure according to formula (Ia): 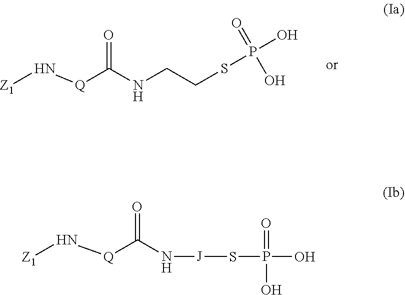
wherein,
NH-Q-C(O) is a peptide chain containing from 2 to 20 amino acids and including a plurality of aromatic amino acids, and the peptide chain optionally comprises a lysine, histidine, or arginine residue having a sidechain covalently bonded to Z₂;
Z₁ is a moiety comprising an aromatic group, a therapeutic agent, a fluorophore, or a nanoparticle; and
Z₂ is H, a therapeutic agent, a fluorophore, or a nanoparticle;
J, if present, is a linker group.

2. A peptide comprising the structure according to formula (II): 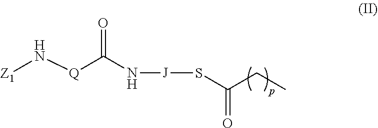
wherein,
NH-Q-C(O) is a peptide chain containing from 2 to 20 amino acids and including a plurality of aromatic amino acids, and the peptide chain optionally comprises a lysine, histidine, or arginine residue having a sidechain covalently bonded to Z₂;
Z₁ is a moiety comprising an aromatic group, a therapeutic agent, a fluorophore, or a nanoparticle;
Z₂ is H, a therapeutic agent, a fluorophore, or a nanoparticle;
p is an integer from 0 to 9; and
J is a linker between the —NH—group covalently attached to the C-terminal end of the peptide chain and the thioester (—SC(O)(CH)ₚCH₃) group.

3. The peptide according to claim 1, wherein Z₁ comprises the aromatic group.

4. The peptide according to claim 3, wherein the aromatic group is selected from the group consisting of phenylacetyl, naphthylacetyl, fluorenylacetyl, pyrenylacetyl, and cinnamoyl.

5. The peptide according to claim 1, wherein the plurality of aromatic amino acids are selected from the group consisting of phenylalanine, tyrosine, and tryptophan.

6. The peptide according to claim 1, wherein the peptide chain comprises at least one cysteine residue.

7. The peptide according to claim 1, wherein Z₁ comprises the fluorophore.

8. The peptide according to claim 7, wherein the fluorophore is 4-nitro-2,1,3-benzoxadiazolyl, 5-(dimethylamino)naphthalene-1-sulfonyl, 4-(N,N-dimethylaminosulfonyl)-2,1,3-benzoxadiazolyl, and 9-acridinyl.

9. The peptide according to claim 1, wherein the peptide chain comprises a lysine, histidine, or arginine residue having a sidechain covalently bonded to Z₂.

10. (canceled)

11. The peptide according to claim 9, wherein Z₂ is H a therapeutic agent or a fluorophore.

12. (canceled)

13. The peptide according to claim 1, wherein the peptide is less than 10 amino acids in length.

14-15. (canceled)

16. The peptide according to claim 1, wherein the peptide chain is selected from the group consisting of FF, FFK, ff, ffk, FFKY (SEQ ID NO: 1), FFFKY (SEQ ID NO: 2), FFGKY (SEQ ID NO: 3), FFGK (SEQ ID NO: 4), FFGKF (SEQ ID NO: 5), ffky, fffky, ffgky, ffgk, ffgkf, FFK(Dmt) (SEQ ID NO: 6), FFFK(Dmt) (SEQ ID NO: 7), FFGK(Dmt) (SEQ ID NO: 8), ffk(dmt), fffk(dmt), ffgk(dmt), FFCY (SEQ ID NO: 9), FFFCY (SEQ ID NO: 10), FFGCY (SEQ ID NO: 11), FFGC (SEQ ID NO: 12), FFGCF (SEQ ID NO:

13. , ffcy, fffcy, ffgcy, ffgc, ffgcf, FFC(Dmt) (SEQ ID NO: 14), FFFC(Dmt) (SEQ ID NO: 15), FFGC(Dmt) (SEQ ID NO: 16), ffc(dmt), fffc(dmt), ffgc(dmt), wherein Dmt is 2,6-dimethyl-L-tyrosine and dmt is 2,6-dimethyl-D-tyrosine.

17. The peptide according to claim 1, which is selected from the group consisting of
Naphthyl-(CH₂)—C(O)—NH—FF—C(O)—NH(CH₂)₂S—P(O)(OH)₂;
NBD-(CH₂)₂—C(O)—NH—FF—C(O)—NH(CH₂)₂S—P(O)(OH)₂;
Naphthyl-(CH₂)—C(O)—NH-ff-C(O)—NH(CH₂)₂S—P(O)(OH)₂;
NBD-(CH₂)₂—C(O)—NH-ff-C(O)—NH(CH₂)₂S—P(O)(OH)₂;
Naphthyl-(CH₂)—C(O)—NH—FFK—C(O)—NH(CH₂)₂S—P(O)(OH)₂;
NBD-(CH₂)₂—C(O)—NH—FFK—C(O)—NH(CH₂)₂S—P(O)(OH)₂;
Naphthyl-(CH₂)—C(O)—NH-ffk-C(O)—NH(CH₂)₂S—P(O)(OH)₂;
NBD-(CH₂)₂—C(O)—NH-ffk-C(O)—NH(CH₂)₂S—P(O)(OH)₂;
Naphthyl-(CH₂)—C(O)—NH—FFKY—C(O)—NH(CH₂)₂S—P(O)(OH)₂;
NBD-(CH₂)₂—C(O)—NH—FFKY—C(O)—NH(CH₂)₂S—P(O)(OH)₂;
Naphthyl-(CH₂)—C(O)—NH-ffky-C(O)—NH(CH₂)₂S—P(O)(OH)₂;
NBD-(CH₂)₂—C(O)—NH-ffky-C(O)—NH(CH₂)₂S—P(O)(OH)₂;
Naphthyl-(CH₂)—C(O)—NH—FFFKY—C(O)—NH(CH₂)₂S—P(O)(OH)₂;
NBD-(CH₂)₂—C(O)—NH—FFFKY—C(O)—NH(CH₂)₂S—P(O)(OH)₂;
Naphthyl-(CH₂)—C(O)—NH-fffky-C(O)—NH(CH₂)₂S—P(O)(OH)₂;
NBD-(CH₂)₂—C(O)—NH-fffky-C(O)—NH(CH₂)₂S—P(O)(OH)₂;
Naphthyl-(CH₂)—C(O)—NH—FFGKY—C(O)—NH(CH₂)₂S—P(O)(OH)₂;
NBD-(CH₂)₂—C(O)—NH—FFGKY—C(O)—NH(CH₂)₂S—P(O)(OH)₂;
Naphthyl-(CH₂)—C(O)—NH-ffgky-C(O)—NH(CH₂)₂S—P(O)(OH)₂;
NBD-(CH₂)₂—C(O)—NH-ffgky-C(O)—NH(CH₂)₂S—P(O)(OH)₂;
Naphthyl-(CH₂)—C(O)—NH—FFGK—C(O)—NH(CH₂)₂S—P(O)(OH)₂;
NBD-(CH₂)₂—C(O)—NH—FFGK—C(O)—NH(CH₂)₂S—P(O)(OH)₂;
Naphthyl-(CH₂)—C(O)—NH-ffgk—C(O)—NH(CH₂)₂S—P(O)(OH)₂;
NBD-(CH₂)₂—C(O)—NH—ffgk—C(O)—NH(CH₂)₂S—P(O)(OH)₂;
Naphthyl-(CH₂)—C(O)—NH—FFGKF—C(O)—NH(CH₂)₂S—P(O)(OH)₂;
NBD-(CH₂)₂—C(O)—NH—FFGKF—C(O)—NH(CH₂)₂S—P(O)(OH)₂;
Naphthyl-(CH₂)—C(O)—NH-ffgkf-C(O)—NH(CH₂)₂S—P(O)(OH)₂;
NBD-(CH₂)₂—C(O)—NH-ffgkf-C(O)—NH(CH₂)₂S—P(O)(OH)₂;
Naphthyl-(CH₂)—C(O)—NH—FFK(Dmt)-C(O)—NH(CH₂)₂S—P(O)(OH)₂;
NBD-(CH₂)₂—C(O)—NH—FFK(Dmt)-C(O)—NH(CH₂)₂S—P(O)(OH)₂;
Naphthyl-(CH₂)—C(O)—NH-ffk(dmt)—C(O)—NH(CH₂)₂S—P(O)(OH)₂;
NBD-(CH₂)₂—C(O)—NH-ffk(dmt)—C(O)—NH(CH₂)₂S—P(O)(OH)₂;
Naphthyl-(CH₂)—C(O)—NH—FFFK(Dmt)-C(O)—NH(CH₂)₂S—P(O)(OH)₂;
NBD-(CH₂)₂—C(O)—NH—FFFK(Dmt)-C(O)—NH(CH₂)₂S—P(O)(OH)₂;
Naphthyl-(CH₂)—C(O)—NH-fffk(dmt)-C(O)—NH(CH₂)₂S—P(O)(OH)₂;
NBD-(CH₂)₂—C(O)—NH-fffk(dmt)-C(O)—NH(CH₂)₂S—P(O)(OH)₂;
Naphthyl-(CH₂)—C(O)—NH—FFGK(Dmt)-C(O)—NH(CH₂)₂S—P(O)(OH)₂;
NBD-(CH₂)₂—C(O)—NH—FFGK(Dmt)-C(O)—NH(CH₂)₂S—P(O)(OH)₂;
Naphthyl-(CH₂)—C(O)—NH-ffgk(dmt)-C(O)—NH(CH₂)₂S—P(O)(OH)₂;
NBD-(CH₂)₂—C(O)—NH-ffgk(dmt)-C(O)—NH(CH₂)₂S—P(O)(OH)₂;
Naphthyl-(CH₂)—C(O)—NH—FFCY—C(O)—NH(CH₂)₂S—P(O)(OH)₂;
NBD-(CH₂)₂—C(O)—NH—FFCY—C(O)—NH(CH₂)₂S—P(O)(OH)₂;
Naphthyl-(CH₂)—C(O)—NH-ffcy-C(O)—NH(CH₂)₂S—P(O)(OH)₂;
NBD-(CH₂)₂—C(O)—NH-ffcy-C(O)—NH(CH₂)₂S—P(O)(OH)₂;
Naphthyl-(CH₂)—C(O)—NH—FFFCY—C(O)—NH(CH₂)₂S—P(O)(OH)₂;
NBD-(CH₂)₂—C(O)—NH—FFFCY—C(O)—NH(CH₂)₂S—P(O)(OH)₂;
Naphthyl-(CH₂)—C(O)—NH-fffcy-C(O)—NH(CH₂)₂S—P(O)(OH)₂;
NBD-(CH₂)₂—C(O)—NH-fffcy-C(O)—NH(CH₂)₂S—P(O)(OH)₂;
Naphthyl-(CH₂)—C(O)—NH—FFGCY—C(O)—NH(CH₂)₂S—P(O)(OH)₂;
NBD-(CH₂)₂—C(O)—NH—FFGCY—C(O)—NH(CH₂)₂S—P(O)(OH)₂;
Naphthyl-(CH₂)—C(O)—NH-ffgcy-C(O)—NH(CH₂)₂S—P(O)(OH)₂;
NBD-(CH₂)₂—C(O)—NH-ffgcy-C(O)—NH(CH₂)₂S—P(O)(OH)₂;
Naphthyl-(CH₂)—C(O)—NH—FFGC—C(O)—NH(CH₂)₂S—P(O)(OH)₂;
NBD-(CH₂)₂—C(O)—NH—FFGC—C(O)—NH(CH₂)₂S—P(O)(OH)₂;
Naphthyl-(CH₂)—C(O)—NH-ffgc-C(O)—NH(CH₂)₂S—P(O)(OH)₂;
NBD-(CH₂)₂—C(O)—NH-ffgc-C(O)—NH(CH₂)₂S—P(O)(OH)₂;
Naphthyl-(CH₂)—C(O)—NH—FFGCF—C(O)—NH(CH₂)₂S—P(O)(OH)₂;
NBD-(CH₂)₂—C(O)—NH—FFGCF—C(O)—NH(CH₂)₂S—P(O)(OH)₂;
Naphthyl-(CH₂)—C(O)—NH-ffgcf-C(O)—NH(CH₂)₂S—P(O)(OH)₂;
NBD-(CH₂)₂—C(O)—NH-ffgcf-C(O)—NH(CH₂)₂S—P(O)(OH)₂;
Naphthyl-(CH₂)—C(O)—NH—FFC(Dmt)-C(O)—NH(CH₂)₂S—P(O)(OH)₂;
NBD-(CH₂)₂—C(O)—NH—FFC(Dmt)-C(O)—NH(CH₂)₂S—P(O)(OH)₂;
Naphthyl-(CH₂)—C(O)—NH-ffc(dmt)-C(O)—NH(CH₂)₂S—P(O)(OH)₂;
NBD-(CH₂)₂—C(O)—NH-ffc(dmt)-C(O)—NH(CH₂)₂S—P(O)(OH)₂;
Naphthyl-(CH₂)—C(O)—NH—FFFC(Dmt)-C(O)—NH(CH₂)₂S—P(O)(OH)₂;
NBD-(CH₂)₂—C(O)—NH—FFFC(Dmt)-C(O)—NH(CH₂)₂S—P(O)(OH)₂;
Naphthyl-(CH₂)—C(O)—NH-fffc(dmt)-C(O)—NH(CH₂)₂S—P(O)(OH)₂;
NBD-(CH₂)₂—C(O)—NH-fffc(dmt)-C(O)—NH(CH₂)₂S—P(O)(OH)₂;
Naphthyl-(CH₂)—C(O)—NH—FFGC(Dmt)-C(O)—NH(CH₂)₂S—P(O)(OH)₂;
NBD-(CH₂)₂—C(O)—NH—FFGC(Dmt)-C(O)—NH(CH₂)₂S—P(O)(OH)₂;
Naphthyl-(CH₂)—C(O)—NH-ffgc(dmt)-C(O)—NH(CH₂)₂S—P(O)(OH)₂;
NBD-(CH₂)₂—C(O)—NH-ffgc(dmt)-C(O)—NH(CH₂)₂S—P(O)(OH)₂;
Naphthyl-(CH₂)—C(O)—NH—FF—C(O)—NH(CH₂)₃NH(CH₂)₂S—P(O)(OH)₂;
NBD-(CH₂)₂—C(O)—NH—FF—C(O)—NH(CH₂)₃NH(CH₂)₂S—P(O)(OH)₂;
Naphthyl-(CH₂)—C(O)—NH-ff-C(O)—NH(CH₂)₃NH(CH₂)₂S—P(O)(OH)₂;
NBD-(CH₂)₂—C(O)—NH-ff-C(O)—NH(CH₂)₃NH(CH₂)₂—P(O)(OH)₂;
Naphthyl-(CH₂)—C(O)—NH—FFK—C(O)—NH(CH₂)₃NH(CH₂)₂S—P(O)(OH)₂;
NBD-(CH₂)₂—C(O)—NH—FFK—C(O)—NH(CH₂)₃NH(CH₂)₂S—P(O)(OH)₂;
Naphthyl-(CH₂)—C(O)—NH-ffk-C(O)—NH(CH₂)₃NH(CH₂)₂S—P(O)(OH)₂;
NBD-(CH₂)₂—C(O)—NH-ffk-C(O)—NH(CH₂)₃NH(CH₂)₂S—P(O)(OH)₂;
Naphthyl-(CH₂)—C(O)—NH—FFKY—C(O)—NH(CH₂)₃NH(CH₂)₂S—P(O)(OH)₂;
NBD-(CH₂)₂—C(O)—NH—FFKY—C(O)—NH(CH₂)₃NH(CH₂)₂S—P(O)(OH)₂;
Naphthyl-(CH₂)—C(O)—NH-ffky-C(O)—NH(CH₂)₃NH(CH₂)₂S—P(O)(OH)₂;
NBD-(CH₂)₂—C(O)—NH-ffky-C(O)—NH(CH₂)₃NH(CH₂)₂S—P(O)(OH)₂;
Naphthyl-(CH₂)—C(O)—NH—FFFKY—C(O)—NH(CH₂)₃NH(CH₂)₂S—P(O)(OH)₂;
NBD-(CH₂)₂—C(O)—NH—FFFKY—C(O)—NH(CH₂)₃NH(CH₂)₂S—P(O)(OH)₂;
Naphthyl-(CH₂)—C(O)—NH-fffky-C(O)—NH(CH₂)₃NH(CH₂)₂S—P(O)(OH)₂;
NBD-(CH₂)₂—C(O)—NH-fffky-C(O)—NH(CH₂)₃NH(CH₂)₂S—P(O)(OH)₂;
Naphthyl-(CH₂)—C(O)—NH—FFGKY—C(O)—NH(CH₂)₃NH(CH₂)₂S—P(O)(OH)₂;
NBD-(CH₂)₂—C(O)—NH—FFGKY—C(O)—NH(CH₂)₃NH(CH₂)₂S—P(O)(OH)₂;
Naphthyl-(CH₂)—C(O)—NH-ffgky-C(O)—NH(CH₂)₃NH(CH₂)₂S—P(O)(OH)₂;
NBD-(CH₂)₂—C(O)—NH-ffgky-C(O)—NH(CH₂)₃NH(CH₂)₂S—P(O)(OH)₂;
Naphthyl-(CH₂)—C(O)—NH—FFGK—C(O)—NH(CH₂)₃NH(CH₂)₂S—P(O)(OH)₂;
NBD-(CH₂)₂—C(O)—NH—FFGK—C(O)—NH(CH₂)₃NH(CH₂)₂S—P(O)(OH)₂;
Naphthyl-(CH₂)—C(O)—NH-ffgk-C(O)—NH(CH₂)₃NH(CH₂)₂S—P(O)(OH)₂;
NBD-(CH₂)₂—C(O)—NH-ffgk-C(O)—NH(CH₂)₃NH(CH₂)₂S—P(O)(OH)₂;
Naphthyl-(CH₂)—C(O)—NH—FFGKF—C(O)—NH(CH₂)₃NH(CH₂)₂S—P(O)(OH)₂;
NBD-(CH₂)₂—C(O)—NH—FFGKF—C(O)—NH(CH₂)₃NH(CH₂)₂S—P(O)(OH)₂;
Naphthyl-(CH₂)—C(O)—NH-ffgkf-C(O)—NH(CH₂)₃NH(CH₂)₂S—P(O)(OH)₂;
NBD-(CH₂)₂—C(O)—NH-ffgkf-C(O)—NH(CH₂)₃NH(CH₂)₂S—P(O)(OH)₂;
Naphthyl-(CH₂)—C(O)—NH—FFK(Dmt)-C(O)—NH(CH₂)₃NH(CH₂)₂S—P(O)(OH)₂;
NBD-(CH₂)₂—C(O)—NH—FFK(Dmt)-C(O)—NH(CH₂)₃NH(CH₂)₂S—P(O)(OH)₂;
Naphthyl-(CH₂)—C(O)—NH-ffk(dmt)-C(O)—NH(CH₂)₃NH(CH₂)₂S—P(O)(OH)₂;
NBD-(CH₂)₂—C(O)—NH-ffk(dmt)-C(O)—NH(CH₂)₃NH(CH₂)₂S—P(O)(OH)₂;
Naphthyl-(CH₂)—C(O)—NH—FFFK(Dmt)-C(O)—NH(CH₂)₃NH(CH₂)₂S—P(O)(OH)₂;
NBD-(CH₂)₂—C(O)—NH—FFFK(Dmt)-C(O)—NH(CH₂)₃NH(CH₂)₂S—P(O)(OH)₂;
Naphthyl-(CH₂)—C(O)—NH-fffk(dmt)—C(O)—NH(CH₂)₃NH(CH₂)₂S—P(O)(OH)₂;
NBD-(CH₂)₂—C(O)—NH-fffk(dmt)-C(O)—NH(CH₂)₃NH(CH₂)₂S—P(O)(OH)₂;
Naphthyl-(CH₂)—C(O)—NH—FFGK(Dmt)-C(O)—NH(CH₂)₃NH(CH₂)₂S—P(O)(OH)₂;
NBD-(CH₂)₂—C(O)—NH—FFGK(Dmt)-C(O)—NH(CH₂)₃NH(CH₂)₂S—P(O)(OH)₂;
Naphthyl-(CH₂)—C(O)—NH-ffgk(dmt)-C(O)—NH(CH₂)₃NH(CH₂)₂S—P(O)(OH)₂;
NBD-(CH₂)₂—C(O)—NH-ffgk(dmt)-C(O)—NH(CH₂)₃NH(CH₂)₂S—P(O)(OH)₂;
Naphthyl-(CH₂)—C(O)—NH—FFCY—C(O)—NH(CH₂)₃NH(CH₂)₂S—P(O)(OH)₂;
NBD-(CH₂)₂—C(O)—NH—FFCY—C(O)—NH(CH₂)₃NH(CH₂)₂S—P(O)(OH)₂;
Naphthyl-(CH₂)—C(O)—NH-ffcy-C(O)—NH(CH₂)₃NH(CH₂)₂S—P(O)(OH)₂;
NBD-(CH₂)₂—C(O)—NH-ffcy-C(O)—NH(CH₂)₃NH(CH₂)₂S—P(O)(OH)₂;
Naphthyl-(CH₂)—C(O)—NH—FFFCY—C(O)—NH(CH₂)₃NH(CH₂)₂S—P(O)(OH)₂;
NBD-(CH₂)₂—C(O)—NH—FFFCY—C(O)—NH(CH₂)₃NH(CH₂)₂S—P(O)(OH)₂;
Naphthyl-(CH₂)—C(O)—NH-fffcy-C(O)—NH(CH₂)₃NH(CH₂)₂S—P(O)(OH)₂;
NBD-(CH₂)₂—C(O)—NH-fffcy-C(O)—NH(CH₂)₃NH(CH₂)₂S—P(O)(OH)₂;
Naphthyl-(CH₂)—C(O)—NH—FFGCY—C(O)—NH(CH₂)₃NH(CH₂)₂S—P(O)(OH)₂;
NBD-(CH₂)₂—C(O)—NH—FFGCY—C(O)—NH(CH₂)₃NH(CH₂)₂S—P(O)(OH)₂;
Naphthyl-(CH₂)—C(O)—NH-ffgcy-C(O)—NH(CH₂)₃NH(CH₂)₂S—P(O)(OH)₂;
NBD-(CH₂)₂—C(O)—NH-ffgcy-C(O)—NH(CH₂)₃NH(CH₂)₂S—P(O)(OH)₂;
Naphthyl-(CH₂)—C(O)—NH—FFGC—C(O)—NH(CH₂)₃NH(CH₂)₂S—P(O)(OH)₂;
NBD-(CH₂)₂—C(O)—NH—FFGC—C(O)—NH(CH₂)₃NH(CH₂)₂S—P(O)(OH)₂;
Naphthyl-(CH₂)—C(O)—NH-ffgc-C(O)—NH(CH₂)₃NH(CH₂)₂S—P(O)(OH)₂;
NBD-(CH₂)₂—C(O)—NH-ffgc-C(O)—NH(CH₂)₃NH(CH₂)₂S—P(O)(OH)₂;
Naphthyl-(CH₂)—C(O)—NH—FFGCF—C(O)—NH(CH₂)₃NH(CH₂)₂S—P(O)(OH)₂;
NBD-(CH₂)₂—C(O)—NH—FFGCF—C(O)—NH(CH₂)₃NH(CH₂)₂S—P(O)(OH)₂;
Naphthyl-(CH₂)—C(O)—NH-ffgcf-C(O)—NH(CH₂)₃NH(CH₂)₂S—P(O)(OH)₂;
NBD-(CH₂)₂—C(O)—NH-ffgcf-C(O)—NH(CH₂)₃NH(CH₂)₂S—P(O)(OH)₂;
Naphthyl-(CH₂)—C(O)—NH—FFC(Dmt)-C(O)—NH(CH₂)₃NH(CH₂)₂S—P(O)(OH)₂;
NBD-(CH₂)₂—C(O)—NH—FFC(Dmt)-C(O)—NH(CH₂)₃NH(CH₂)₂S—P(O)(OH)₂;
Naphthyl-(CH₂)—C(O)—NH-ffc(dmt)-C(O)—NH(CH₂)₃NH(CH₂)₂S—P(O)(OH)₂;
NBD-(CH₂)₂—C(O)—NH-ffc(dmt)-C(O)—NH(CH₂)₃NH(CH₂)₂S—P(O)(OH)₂;
Naphthyl-(CH₂)—C(O)—NH—FFFC(Dmt)-C(O)—NH(CH₂)₃NH(CH₂)₂S—P(O)(OH)₂;
NBD-(CH₂)₂—C(O)—NH—FFFC(Dmt)-C(O)—NH(CH₂)₃NH(CH₂)₂S—P(O)(OH)₂;
Naphthyl-(CH₂)—C(O)—NH-fffc(dmt)-C(O)—NH(CH₂)₃NH(CH₂)₂S—P(O)(OH)₂;
NBD-(CH₂)₂—C(O)—NH-fffc(dmt)-C(O)—NH(CH₂)₃NH(CH₂)₂S—P(O)(OH)₂;
Naphthyl-(CH₂)—C(O)—NH—FFGC(Dmt)-C(O)—NH(CH₂)₃NH(CH₂)₂S—P(O)(OH)₂;
NBD-(CH₂)₂—C(O)—NH—FFGC(Dmt)-C(O)—NH(CH₂)₃NH(CH₂)₂S—P(O)(OH)₂;
Naphthyl-(CH₂)—C(O)—NH-ffgc(dmt)-C(O)—NH(CH₂)₃NH(CH₂)₂S—P(O)(OH)₂;
NBD-(CH₂)₂—C(O)—NH-ffgc(dmt)-C(O)—NH(CH₂)₃NH(CH₂)₂S—P(O)(OH)₂; wherein NBD is 4-nitro-2,1,3-benzoxadiazolyl; Dmt is 2,6-dimethyl-L-tyrosine; and dmt is 2,6-dimethyl-D-tyrosine.

18. The peptide according to claim 1, which is selected from the group consisting of
Naphthyl-CH₂C(O)—NH—FFK(ε-Z₂)—C(O)—NH(CH₂)₂S—P(O)(OH)₂;
Naphthyl-CH₂C(O)—NH-ffk(ε-Z₂)—C(O)—NH(CH₂)₂S—P(O)(OH)₂;
Naphthyl-CH₂C(O)—NH—FFK(ε-Z₂)Y—C(O)—NH(CH₂)₂S—P(O)(OH)₂;
Naphthyl-CH₂C(O)—NH-ffk(ε-Z₂)y-C(O)—NH(CH₂)₂S—P(O)(OH)₂;
Naphthyl-CH₂C(O)—NH—FFFK(ε-Z₂)Y—C(O)—NH(CH₂)₂S—P(O)(OH)₂;
Naphthyl-CH₂C(O)—NH-fffk(ε-Z₂)y-C(O)—NH(CH₂)₂S—P(O)(OH)₂;
Naphthyl-CH₂C(O)—NH—FFGK(ε-Z₂)Y—C(O)—NH(CH₂)₂S—P(O)(OH)₂;
Naphthyl-CH₂C(O)—NH-ffgk(ε-Z₂)y-C(O)—NH(CH₂)₂S—P(O)(OH)₂;
Naphthyl-CH₂C(O)—NH—FFGK(ε-Z₂)—C(O)—NH(CH₂)₂S—P(O)(OH)₂;
Naphthyl-CH₂C(O)—NH-ffgk(ε-Z₂)—C(O)—NH(CH₂)₂S—P(O)(OH)₂;
Naphthyl-CH₂C(O)—NH—FFGK(ε-Z₂)F—C(O)—NH(CH₂)₂S—P(O)(OH)₂;
Naphthyl-CH₂C(O)—NH-ffgk(ε-Z₂)f-C(O)—NH(CH₂)₂S—P(O)(OH)₂;
Naphthyl-CH₂C(O)—NH—FFK(ε-Z₂)(Dmt)-C(O)—NH(CH₂)₂S—P(O)(OH)₂;
Naphthyl-CH₂C(O)—NH-ffk(ε-Z₂)(dmt)-C(O)—NH(CH₂)₂S—P(O)(OH)₂;
Naphthyl-CH₂C(O)—NH—FFFK(ε-Z₂)(Dmt)-C(O)—NH(CH₂)₂S—P(O)(OH)₂;
Naphthyl-CH₂C(O)—NH-fffk(ε-Z₂)(dmt)-C(O)—NH(CH₂)₂S—P(O)(OH)₂;
Naphthyl-CH₂C(O)—NH—FFGK(ε-Z₂)(Dmt)-C(O)—NH(CH₂)₂S—P(O)(OH)₂;
Naphthyl-CH₂C(O)—NH-ffgk(ε-Z₂)(dmt)-C(O)—NH(CH₂)₂S—P(O)(OH)₂;
NBD-(CH₂)₂C(O)—NH—FFK(ε-Z₂)—C(O)—NH(CH₂)₂S—P(O)(OH)₂;
NBD-(CH₂)₂C(O)—NH-ffk(ε-Z₂)—C(O)—NH(CH₂)₂S—P(O)(OH)₂;
NBD-(CH₂)₂C(O)—NH—FFK(ε-Z₂)Y—C(O)—NH(CH₂)₂S—P(O)(OH)₂;
NBD-(CH₂)₂C(O)—NH-ffk(ε-Z₂)y-C(O)—NH(CH₂)₂S—P(O)(OH)₂;
NBD-(CH₂)₂C(O)—NH—FFFK(ε-Z₂)Y—C(O)—NH(CH₂)₂S—P(O)(OH)₂;
NBD-(CH₂)₂C(O)—NH-fffk(ε-Z₂)y-C(O)—NH(CH₂)₂S—P(O)(OH)₂;
NBD-(CH₂)₂C(O)—NH—FFGK(ε-Z₂)Y—C(O)—NH(CH₂)₂S—P(O)(OH)₂;
NBD-(CH₂)₂C(O)—NH-ffgk(ε-Z₂)y-C(O)—NH(CH₂)₂S—P(O)(OH)₂;
NBD-(CH₂)₂C(O)—NH—FFGK(ε-Z₂)—C(O)—NH(CH₂)₂S—P(O)(OH)₂;
NBD-(CH₂)₂C(O)—NH-ffgk(ε-Z₂)—C(O)—NH(CH₂)₂S—P(O)(OH)₂;
NBD-(CH₂)₂C(O)—NH—FFGK(ε-Z₂)F—C(O)—NH(CH₂)₂S—P(O)(OH)₂;
NBD-(CH₂)₂C(O)—NH-ffgk(ε-Z₂)f-C(O)—NH(CH₂)₂S—P(O)(OH)₂;
NBD-(CH₂)₂C(O)—NH—FFK(ε-Z₂)(Dmt)-C(O)—NH(CH₂)₂S—P(O)(OH)₂;
NBD-(CH₂)₂C(O)—NH-ffk(ε-Z₂)(dmt)-C(O)—NH(CH₂)₂S—P(O)(OH)₂;
NBD-(CH₂)₂C(O)—NH—FFFK(ε-Z₂)(Dmt)-C(O)—NH(CH₂)₂S—P(O)(OH)₂;
NBD-(CH₂)₂C(O)—NH-fffk(ε-Z₂)(dmt)-C(O)—NH(CH₂)₂S—P(O)(OH)₂;
NBD-(CH₂)₂C(O)—NH—FFGK(ε-Z₂)(Dmt)-C(O)—NH(CH₂)₂S—P(O)(OH)₂;
NBD-(CH₂)₂C(O)—NH-ffgk(ε-Z₂)(dmt)-C(O)—NH(CH₂)₂S—P(O)(OH)₂;
Naphthyl-CH₂C(O)—NH—FFK(ε-Z₂)—C(O)—NH(CH₂)₃NH(CH₂)₂S—P(O)(OH)₂;
Naphthyl-CH₂C(O)—NH—ffk(ε-Z₂)—C(O)—NH(CH₂)₃NH(CH₂)₂S—P(O)(OH)₂;
Naphthyl-CH₂C(O)—NH—FFK(ε-Z₂)Y—C(O)—NH(CH₂)₃NH(CH₂)₂S—P(O)(OH)₂;
Naphthyl-CH₂C(O)—NH—ffk(ε-Z₂)y-C(O)—NH(CH₂)₃NH(CH₂)₂S—P(O)(OH)₂;
Naphthyl-CH₂C(O)—NH—FFFK(ε-Z₂)Y—C(O)—NH(CH₂)₃NH(CH_{2 2}S—P(O)(OH)₂;
Naphthyl-CH₂C(O)—NH—fffk(ε-Z₂)y-C(O)—NH(CH₂)₃NH(CH₂)₂S—P(O)(OH)₂;
Naphthyl-CH₂C(O)—NH—FFGK(ε-Z₂)Y—C(O)—NH(CH₂)₃NH(CH_{2 2}S—P(O)(OH)₂;
Naphthyl-CH₂C(O)—NH—ffgk(ε-Z₂)y-C(O)—NH(CH₂)₃NH(CH₂)₂S—P(O)(OH)₂;
Naphthyl-CH₂C(O)—NH—FFGK(ε-Z₂)—C(O)—NH(CH₂)₃NH(CH₂)₂S—P(O)(OH)₂;
Naphthyl-CH₂C(O)—NH-ffgk(ε-Z₂)—C(O)—NH(CH₂)₃NH(CH₂)₂S—P(O)(OH)₂;
Naphthyl-CH₂C(O)—NH—FFGK(ε-Z₂)F—C(O)—NH(CH₂)₃NH(CH₂)₂S—P(O)(OH)₂;
Naphthyl-CH₂C(O)—NH-ffgk(ε-Z₂)f—C(O)—NH(CH₂)₃NH(CH₂)₂S—P(O)(OH)₂;
Naphthyl-CH₂C(O)—NH—FFK(ε-Z₂)(Dmt)-C(O)—NH(CH₂)₃NH(CH₂)₂S—P(O)(OH)₂;
Naphthyl-CH₂C(O)—NH-ffk(ε-Z₂)(dmt)—C(O)—NH(CH₂)₃NH(CH₂)₂S—P(O)(OH)₂;
Naphthyl-CH₂C(O)—NH—FFFK(ε-Z₂)(Dmt)-C(O)—NH(CH₂)₃NH(CH₂)₂S—P(O)(OH)₂;
Naphthyl-CH₂C(O)—NH-fffk(ε-Z₂)(dmt)—C(O)—NH(CH₂)₃NH(CH₂)₂S—P(O)(OH)₂;
Naphthyl-CH₂C(O)—NH—FFGK(ε-Z₂)(Dmt)-C(O)—NH(CH₂)₃NH(CH₂)₂S—P(O)(OH)₂;
Naphthyl-CH₂C(O)—NH-ffgk(ε-Z₂)(dmt)—C(O)—NH(CH₂)₃NH(CH₂)₂S—P(O)(OH)₂;
NBD-(CH₂)₂—C(O)—NH—FFK(ε-Z₂)—C(O)—NH(CH₂)₃NH(CH₂)₂S—P(O)(OH)₂;
NBD-(CH₂)₂—C(O)—NH-ffk(ε-Z₂)—C(O)—NH(CH₂)₃NH(CH₂)₂S—P(O)(OH)₂;
NBD-(CH₂)₂—C(O)—NH—FFK(ε-Z₂)Y—C(O)—NH(CH₂)₃NH(CH₂)₂S—P(O)(OH)₂;
NBD-(CH₂)₂—C(O)—NH-ffk(ε-Z₂)y-C(O)—NH(CH₂)₃NH(CH₂)₂S—P(O)(OH)₂;
NBD-(CH₂)₂—C(O)—NH—FFFK(ε-Z₂)Y—C(O)—NH(CH₂)₃NH(CH₂)₂S—P(O)(OH)₂;
NBD-(CH₂)₂—C(O)—NH-fffk(ε-Z₂)y-C(O)—NH(CH₂)₃NH(CH₂)₂S—P(O)(OH)₂;
NBD-(CH₂)₂—C(O)—NH—FFGK(ε-Z₂)Y—C(O)—NH(CH₂)₃NH(CH₂)₂S—P(O)(OH)₂;
NBD-(CH₂)₂—C(O)—NH-ffgk(ε-Z₂)y-C(O)—NH(CH₂)₃NH(CH₂)₂S—P(O)(OH)₂;
NBD-(CH₂)₂—C(O)—NH—FFGK(ε-Z₂)—C(O)—NH(CH₂)₃NH(CH₂)₂S—P(O)(OH)₂;
NBD-(CH₂)₂—C(O)—NH-ffgk(ε-Z₂)—C(O)—NH(CH₂)₃NH(CH₂)₂S—P(O)(OH)₂;
NBD-(CH₂)₂—C(O)—NH—FFGK(ε-Z₂)F—C(O)—NH(CH₂)₃NH(CH₂)₂S—P(O)(OH)₂;
NBD-(CH₂)₂—C(O)—NH-ffgk(ε-Z₂)f—C(O)—NH(CH₂)₃NH(CH₂)₂S—P(O)(OH)₂;
NBD-(CH₂)₂—C(O)—NH—FFK(ε-Z₂)(Dmt)-C(O)—NH(CH₂)₃NH(CH₂)₂S—P(O)(OH)₂;
NBD-(CH₂)₂—C(O)—NH-ffk(ε-Z₂)(dmt)—C(O)—NH(CH₂)₃NH(CH₂)₂S—P(O)(OH)₂;
NBD-(CH₂)₂—C(O)—NH—FFFK(ε-Z₂)(Dmt)-C(O)—NH(CH₂)₃NH(CH₂)₂S—P(O)(OH)₂;
NBD-(CH₂)₂—C(O)—NH-fffk(ε-Z₂)(dmt)—C(O)—NH(CH₂)₃NH(CH₂)₂S—P(O)(OH)₂;
NBD-(CH₂)₂—C(O)—NH—FFGK(ε-Z₂)(Dmt)-C(O)—NH(CH₂)₃NH(CH₂)₂S—P(O)(OH)₂;
NBD-(CH₂)₂—C(O)—NH-ffgk(ε-Z₂)(dmt)—C(O)—NH(CH₂)₃NH(CH₂)₂S—P(O)(OH)₂;
wherein Dmt is 2,6-dimethyl-L-tyrosine; dmt is 2,6-dimethyl-D-tyrosine; NBD is 4-nitro-2,1,3-benzoxadiazolyl.

19-20. (canceled)

21. The peptide according to claim 2, which is selected from the group consisting of
Naphthyl-(CH₂)—C(O)—NH—FF—C(O)—NH(CH₂)₂S—C(O)CH₃;
NBD-(CH₂)₂—C(O)—NH—FF—C(O)—NH(CH₂)₂S—C(O)CH₃;
Naphthyl-(CH₂)—C(O)—NH-ff-C(O)—NH(CH₂)₂S—C(O)CH₃;
NBD-(CH₂)₂—C(O)—NH-ff-C(O)—NH(CH₂)₂S—C(O)CH₃;
Naphthyl-(CH₂)—C(O)—NH—FFK—C(O)—NH(CH₂)₂S—C(O)CH₃;
NBD-(CH₂)₂—C(O)—NH—FFK—C(O)—NH(CH₂)₂S—C(O)CH₃;
Naphthyl-(CH₂)—C(O)—NH-ffk-C(O)—NH(CH₂)₂S—C(O)CH₃;
NBD-(CH₂)₂—C(O)—NH-ffk-C(O)—NH(CH₂)₂S—C(O)CH₃;
Naphthyl-(CH₂)—C(O)—NH—FFKY—C(O)—NH(CH₂)₂S—C(O)CH₃;
NBD-(CH₂)₂—C(O)—NH—FFKY—C(O)—NH(CH₂)₂S—C(O)CH₃;
Naphthyl-(CH₂)—C(O)—NH-ffky-C(O)—NH(CH₂)₂S—C(O)CH₃;
NBD-(CH₂)₂—C(O)—NH-ffky-C(O)—NH(CH₂)₂S—C(O)CH₃;
Naphthyl-(CH₂)—C(O)—NH—FFFKY—C(O)—NH(CH₂)₂S—C(O)CH₃;
NBD-(CH₂)₂—C(O)—NH—FFFKY—C(O)—NH(CH₂)₂S—C(O)CH₃;
Naphthyl-(CH₂)—C(O)—NH-fffky-C(O)—NH(CH₂)₂S—C(O)CH₃;
NBD-(CH₂)₂—C(O)—NH-fffky-C(O)—NH(CH₂)₂S—C(O)CH₃;
Naphthyl-(CH₂)—C(O)—NH—FFGKY—C(O)—NH(CH₂)₂S—C(O)CH₃;
NBD-(CH₂)₂—C(O)—NH—FFGKY—C(O)—NH(CH₂)₂S—C(O)CH₃;
Naphthyl-(CH₂)—C(O)—NH-ffgky-C(O)—NH(CH₂)₂S—C(O)CH₃;
NBD-(CH₂)₂—C(O)—NH-ffgky-C(O)—NH(CH₂)₂S—C(O)CH₃;
Naphthyl-(CH₂)—C(O)—NH—FFGK—C(O)—NH(CH₂)₂S—C(O)CH₃;
NBD-(CH₂)₂—C(O)—NH—FFGK—C(O)—NH(CH₂)₂S—C(O)CH₃;
Naphthyl-(CH₂)—C(O)—NH-ffgk—C(O)—NH(CH₂)₂S—C(O)CH₃;
NBD-(CH₂)₂—C(O)—NH-ffgk-C(O)—NH(CH₂)₂S—C(O)CH₃;
Naphthyl-(CH₂)—C(O)—NH—FFGKF—C(O)—NH(CH₂)₂S—C(O)CH₃;
NBD-(CH₂)₂—C(O)—NH—FFGKF—C(O)—NH(CH₂)₂S—C(O)CH₃;
Naphthyl-(CH₂)—C(O)—NH-ffgkf-C(O)—NH(CH₂)₂S—C(O)CH₃;
NBD-(CH₂)₂—C(O)—NH-ffgkf-C(O)—NH(CH₂)₂S—C(O)CH₃;
Naphthyl-(CH₂)—C(O)—NH—FFK(Dmt)-C(O)—NH(CH₂)₂S—C(O)CH₃;
NBD-(CH₂)₂—C(O)—NH—FFK(Dmt)-C(O)—NH(CH₂)₂S—C(O)CH₃;
Naphthyl-(CH₂)—C(O)—NH-ffk(dmt)-C(O)—NH(CH₂)₂S—C(O)CH₃;
NBD-(CH₂)₂—C(O)—NH-ffk(dmt)-C(O)—NH(CH₂)₂S—C(O)CH₃;
Naphthyl-(CH₂)—C(O)—NH—FFFK(Dmt)-C(O)—NH(CH₂)₂S—C(O)CH₃;
NBD-(CH₂)₂—C(O)—NH—FFFK(Dmt)-C(O)—NH(CH₂)₂S—C(O)CH₃;
Naphthyl-(CH₂)—C(O)—NH-fffk(dmt)-C(O)—NH(CH₂)₂S—C(O)CH₃;
NBD-(CH₂)₂—C(O)—NH-fffk(dmt)-C(O)—NH(CH₂)₂S—C(O)CH₃;
Naphthyl-(CH₂)—C(O)—NH—FFGK(Dmt)-C(O)—NH(CH₂)₂S—C(O)CH₃;
NBD-(CH₂)₂—C(O)—NH—FFGK(Dmt)-C(O)—NH(CH₂)₂S—C(O)CH₃;
Naphthyl-(CH₂)—C(O)—NH-ffgk(dmt)-C(O)—NH(CH₂)₂S—C(O)CH₃;
NBD-(CH₂)₂—C(O)—NH-ffgk(dmt)-C(O)—NH(CH₂)₂S—C(O)CH₃;
Naphthyl-(CH₂)—C(O)—NH—FFCY—C(O)—NH(CH₂)₂S—C(O)CH₃;
NBD-(CH₂)₂—C(O)—NH—FFCY—C(O)—NH(CH₂)₂S—C(O)CH₃;
Naphthyl-(CH₂)—C(O)—NH-ffcy-C(O)—NH(CH₂)₂S—C(O)CH₃;
NBD-(CH₂)₂—C(O)—NH-ffcy-C(O)—NH(CH₂)₂S—C(O)CH₃;
Naphthyl-(CH₂)—C(O)—NH—FFFCY—C(O)—NH(CH₂)₂S—C(O)CH₃;
NBD-(CH₂)₂—C(O)—NH—FFFCY—C(O)—NH(CH₂)₂S—C(O)CH₃;
Naphthyl-(CH₂)—C(O)—NH-fffcy-C(O)—NH(CH₂)₂S—C(O)CH₃;
NBD-(CH₂)₂—C(O)—NH-fffcy-C(O)—NH(CH₂)₂S—C(O)CH₃;
Naphthyl-(CH₂)—C(O)—NH—FFGCY—C(O)—NH(CH₂)₂S—C(O)CH₃;
NBD-(CH₂)₂—C(O)—NH—FFGCY—C(O)—NH(CH₂)₂S—C(O)CH₃;
Naphthyl-(CH₂)—C(O)—NH-ffgcy-C(O)—NH(CH₂)₂S—C(O)CH₃;
NBD-(CH₂)₂—C(O)—NH-ffgcy-C(O)—NH(CH₂)₂S—C(O)CH₃;
Naphthyl-(CH₂)—C(O)—NH—FFGC—C(O)—NH(CH₂)₂S—C(O)CH₃;
NBD-(CH₂)₂—C(O)—NH—FFGC—C(O)—NH(CH₂)₂S—C(O)CH₃;
Naphthyl-(CH₂)—C(O)—NH-ffgc-C(O)—NH(CH₂)₂S—C(O)CH₃;
NBD-(CH₂)₂—C(O)—NH-ffgc-C(O)—NH(CH₂)₂S—C(O)CH₃;
Naphthyl-(CH₂)—C(O)—NH—FFGCF—C(O)—NH(CH₂)₂S—C(O)CH₃;
NBD-(CH₂)₂—C(O)—NH—FFGCF—C(O)—NH(CH₂)₂S—C(O)CH₃;
Naphthyl-(CH₂)—C(O)—NH-ffgcf-C(O)—NH(CH₂)₂S—C(O)CH₃;
NBD-(CH₂)₂—C(O)—NH-ffgcf-C(O)—NH(CH₂)₂S—C(O)CH₃;
Naphthyl-(CH₂)—C(O)—NH—FFC(Dmt)-C(O)—NH(CH₂)₂S—C(O)CH₃;
NBD-(CH₂)₂—C(O)—NH—FFC(Dmt)-C(O)—NH(CH₂)₂S—C(O)CH₃;
Naphthyl-(CH₂)—C(O)—NH-ffc(dmt)-C(O)—NH(CH₂)₂S—C(O)CH₃;
NBD-(CH₂)₂—C(O)—NH-ffc(dmt)-C(O)—NH(CH₂)₂S—C(O)CH₃;
Naphthyl-(CH₂)—C(O)—NH—FFFC(Dmt)-C(O)—NH(CH₂)₂S—C(O)CH₃;
NBD-(CH₂)₂—C(O)—NH—FFFC(Dmt)-C(O)—NH(CH₂)₂S—C(O)CH₃;
Naphthyl-(CH₂)—C(O)—NH-fffc(dmt)-C(O)—NH(CH₂)₂S—C(O)CH₃;
NBD-(CH₂)₂—C(O)—NH-fffc(dmt)-C(O)—NH(CH₂)₂S—C(O)CH₃;
Naphthyl-(CH₂)—C(O)—NH—FFGC(Dmt)-C(O)—NH(CH₂)₂S—C(O)CH₃;
NBD-(CH₂)₂—C(O)—NH—FFGC(Dmt)-C(O)—NH(CH₂)₂S—C(O)CH₃;
Naphthyl-(CH₂)—C(O)—NH-ffgc(dmt)-C(O)—NH(CH₂)₂S—C(O)CH₃;
NBD-(CH₂)₂—C(O)—NH-ffgc(dmt)-C(O)—NH(CH₂)₂S—C(O)CH₃;
Naphthyl-CH₂C(O)—NH—FFK(ε-Z₂)—C(O)—NH(CH₂)₂S—C(O)CH₃;
Naphthyl-CH₂C(O)—NH-ffk(ε-Z₂)—C(O)—NH(CH₂)₂S—C(O)CH₃;
Naphthyl-CH₂C(O)—NH—FFK(ε-Z₂)Y—C(O)—NH(CH₂)₂S—C(O)CH₃;
Naphthyl-CH₂C(O)—NH-ffk(ε-Z₂)y-C(O)—NH(CH₂)₂S—C(O)CH₃;
Naphthyl-CH₂C(O)—NH—FFFK(ε-Z₂)Y—C(O)—NH(CH₂)₂S—C(O)CH₃;
Naphthyl-CH₂C(O)—NH-fffk(ε-Z₂)y-C(O)—NH(CH₂)₂S—C(O)CH₃;
Naphthyl-CH₂C(O)—NH—FFGK(ε-Z₂)Y—C(O)—NH(CH₂)₂S—C(O)CH₃;
Naphthyl-CH₂C(O)—NH-ffgk(ε-Z₂)y-C(O)—NH(CH₂)₂S—C(O)CH₃;
Naphthyl-CH₂C(O)—NH—FFGK(ε-Z₂)—C(O)—NH(CH₂)₂S—C(O)CH₃;
Naphthyl-CH₂C(O)—NH-ffgk(ε-Z₂)—C(O)—NH(CH₂)₂S—C(O)CH₃;
Naphthyl-CH₂C(O)—NH—FFGK(ε-Z₂)F—C(O)—NH(CH₂)₂S—C(O)CH₃;
Naphthyl-CH₂C(O)—NH-ffgk(ε-Z₂)f-C(O)—NH(CH₂)₂S—C(O)CH₃;
Naphthyl-CH₂C(O)—NH—FFK(ε-Z₂)(Dmt)-C(O)—NH(CH₂)₂S—C(O)CH₃;
Naphthyl-CH₂C(O)—NH-ffk(ε-Z₂)(dmt)-C(O)—NH(CH₂)₂S—C(O)CH₃;
Naphthyl-CH₂C(O)—NH—FFFK(ε-Z₂)(Dmt)-C(O)—NH(CH₂)₂S—C(O)CH₃;
Naphthyl-CH₂C(O)—NH-fffk(ε-Z₂)(dmt)-C(O)—NH(CH₂)₂S—C(O)CH₃;
Naphthyl-CH₂C(O)—NH—FFGK(ε-Z₂)(Dmt)-C(O)—NH(CH₂)₂S—C(O)CH₃;
Naphthyl-CH₂C(O)—NH-ffgk(ε-Z₂)(dmt)-C(O)—NH(CH₂)₂S—C(O)CH₃;
NBD-(CH₂)₂C(O)—NH—FFK(ε-Z₂)—C(O)—NH(CH₂)₂S—C(O)CH₃;
NBD-(CH₂)₂C(O)—NH-ffk(ε-Z₂)—C(O)—NH(CH₂)₂S—C(O)CH₃;
NBD-(CH₂)₂C(O)—NH—FFK(ε-Z₂)Y—C(O)—NH(CH₂)₂S—C(O)CH₃;
NBD-(CH₂)₂C(O)—NH-ffk(ε-Z₂)y-C(O)—NH(CH₂)₂S—C(O)CH₃;
NBD-(CH₂)₂C(O)—NH—FFFK(ε-Z₂)Y—C(O)—NH(CH₂)₂S—C(O)CH₃;
NBD-(CH₂)₂C(O)—NH-fffk(ε-Z₂)y-C(O)—NH(CH₂)₂S—C(O)CH₃;
NBD-(CH₂)₂C(O)—NH—FFGK(ε-Z₂)Y—C(O)—NH(CH₂)₂S—C(O)CH₃;
NBD-(CH₂)₂C(O)—NH-ffgk(ε-Z₂)y-C(O)—NH(CH₂)₂S—C(O)CH₃;
NBD-(CH₂)₂C(O)—NH—FFGK(ε-Z₂)—C(O)—NH(CH₂)₂S—C(O)CH₃;
NBD-(CH₂)₂C(O)—NH-ffgk(ε-Z₂)—C(O)—NH(CH₂)₂S—C(O)CH₃;
NBD-(CH₂)₂C(O)—NH—FFGK(ε-Z₂)F—C(O)—NH(CH₂)₂S—C(O)CH₃;
NBD-(CH₂)₂C(O)—NH-ffgk(ε-Z₂)f-C(O)—NH(CH₂)₂S—C(O)CH₃;
NBD-(CH₂)₂C(O)—NH—FFK(ε-Z₂)(Dmt)-C(O)—NH(CH₂)₂S—C(O)CH₃;
NBD-(CH₂)₂C(O)—NH-ffk(ε-Z₂)(dmt)-C(O)—NH(CH₂)₂S—C(O)CH₃;
NBD-(CH₂)₂C(O)—NH—FFFK(ε-Z₂)(Dmt)-C(O)—NH(CH₂)₂S—C(O)CH₃;
NBD-(CH₂)₂C(O)—NH-fffk(ε-Z₂)(dmt)- C(O)—NH(CH₂)₂S—C(O)CH₃;
NBD-(CH₂)₂C(O)—NH—FFGK(ε-Z₂)(Dmt)-C(O)—NH(CH₂)₂S—C(O)CH₃;
NBD-(CH₂)₂C(O)—NH-ffgk(ε-Z₂)(dmt)-C(O)—NH(CH₂)₂S—C(O)CH₃;
wherein Dmt is 2,6-dimethyl-L-tyrosine; dmt is 2,6-dimethyl-D-tyrosine; and NBD is 4-nitro-2,1,3-benzoxadiazolyl; and Z2 is defined herein.

22. The peptide according to claim 1, wherein Z₁ or Z₂ is selected from the group consisting of antioxidants, coenzymes, vitamins, metabolites, proteins or polypeptides, analgesics, anti-inflammatory agents, antihelminthics, anti-arrhythmic agents, anti-bacterial agents, anti-viral agents, anti-coagulants, anti-depressants, anti-diabetics, anti-epileptics, anti-fungal agents, anti-gout agents, anti-hypertensive agents, anti-thrombogenic agents, anti-claudication agents, anti-atherosclerotic drugs, vascular agents, anti-malarials, anti-migraine agents, anti-muscarinic agents, anti-neoplastic agents, erectile dysfunction improvement agents, immunosuppressants, anti-protozoal agents, anti-thyroid agents, anxiolytic agents, sedatives, hypnotics, neuroleptics, β-blockers, cardiac inotropic agents, corticosteroids, diuretics, anti-parkinsonian agents, gastro-intestinal agents, histamine receptor antagonists, keratolyptics, lipid regulating agents, anti-anginal agents, Cox-2 inhibitors, leukotriene inhibitors, macrolides, muscle relaxants, nutritional agents, opioid analgesics, protease inhibitors, sex hormones, stimulants, anti-osteoporosis agents, anti-obesity agents, cognition enhancers, anti-urinary incontinence agents, anti-benign prostate hypertrophy agents, essential fatty acids, non-essential fatty acids, cytokines, growth factors, antibodies, radioprotective agents, and cardioprotective agents.

23. The peptide according to any claim 11, wherein Z₂ comprises 4-nitro-2,1,3-benzoxadiazolyl, 5-(dimethylamino)naphthalene-1-sulfonyl, 4-(N,N-dimethylaminosulfonyl)-2,1,3-benzoxadiazolyl, or 9-acridinyl.

24. A pharmaceutical composition comprising the peptide according to claim 1 in an aqueous medium.

25. The pharmaceutical composition according to claim 24, wherein the peptide forms micelle structures, and comprising a therapeutic agent encapsulated within the micelle structures.

26-27. (canceled)

28. The pharmaceutical composition according to claim 25, wherein the therapeutic agent is selected from the group consisting of analgesics, anti-inflammatory agents, antihelminthics, anti-arrhythmic agents, anti-bacterial agents, anti-viral agents, anti-coagulants, anti-depressants, anti-diabetics, anti-epileptics, anti-fungal agents, anti-gout agents, anti-hypertensive agents, anti-thrombogenic agents, anti-claudication agents, anti-atherosclerotic drugs, vascular agents, anti-malarials, anti-migraine agents, anti-muscarinic agents, anti-neoplastic agents (e.g., antiproliferative or chemotherapeutic agents), erectile dysfunction improvement agents, immunosuppressants, anti-protozoal agents, anti-thyroid agents, anxiolytic agents, sedatives, hypnotics, neuroleptics, β-blockers, cardiac inotropic agents, corticosteroids, diuretics, anti-parkinsonian agents, gastro-intestinal agents, histamine receptor antagonists, keratolyptics, lipid regulating agents, anti-anginal agents, Cox-2 inhibitors, leukotriene inhibitors, macrolides, muscle relaxants, nutritional agents, opioid analgesics, protease inhibitors, sex hormones, stimulants, anti-osteoporosis agents, anti-obesity agents, cognition enhancers, anti-urinary incontinence agents, anti-benign prostate hypertrophy agents, essential fatty acids, non-essential fatty acids, antioxidants, and mixtures thereof.

29-37. (canceled)

38. A method of delivering a therapeutic agent into the Golgi apparatus comprising:
encapsulating a therapeutic agent within a micelle structure of a pharmaceutical composition according to claim 24; and
contacting a cell with the pharmaceutical composition, whereby micelle structures are taken up by the cell and targeted to the Golgi apparatus within the cell.

39. A method of delivering a drug moiety into the Golgi apparatus comprising:
providing a peptide according to claim 1, wherein Z₁ or Z₂ is a drug moiety, or a composition comprising the peptide; and
contacting a cell with the peptide or the composition, whereby the peptide or micelle structures formed by the peptide is taken up by the cell and targeted to the Golgi apparatus within the cell.

40-42. (canceled)

43. A nanofiber or supramolecular hydrogel formed in an aqueous medium and comprising a self-assembled, dephosphorylated form of the peptide according to claim 1.

44. (canceled)

45. The nanofiber or supramolecular hydrogel according to claim 43, wherein the dephosphorylated form of the peptide comprises one of:
Naphthyl-(CH₂)—C(O)—NH—FF—C(O)—NH(CH₂)₂SH;
NBD-(CH₂)₂—C(O)—NH—FF—C(O)—NH(CH₂)₂SH;
Naphthyl-(CH₂)—C(O)—NH-ff-C(O)—NH(CH₂)₂SH;
NBD-(CH₂)₂—C(O)—NH-ff-C(O)—NH(CH₂)₂SH;
Naphthyl-(CH₂)—C(O)—NH—FFK—C(O)—NH(CH₂)₂SH;
NBD-(CH₂)₂—C(O)—NH—FFK—C(O)—NH(CH₂)₂SH;
Naphthyl-(CH₂)—C(O)—NH-ffk-C(O)—NH(CH₂)₂SH;
NBD-(CH₂)₂—C(O)—NH-ffk-C(O)—NH(CH₂)₂SH;
Naphthyl-(CH₂)—C(O)—NH—FFKY—C(O)—NH(CH₂)₂SH;
NBD-(CH₂)₂—C(O)—NH—FFKY—C(O)—NH(CH₂)₂SH;
Naphthyl-(CH₂)—C(O)—NH-ffky-C(O)—NH(CH₂)₂SH;
NBD-(CH₂)₂—C(O)—NH-ffky-C(O)—NH(CH₂)₂SH;
Naphthyl-(CH₂)—C(O)—NH—FFFKY—C(O)—NH(CH₂)₂SH;
NBD-(CH₂)₂—C(O)—NH—FFFKY—C(O)—NH(CH₂)₂SH;
Naphthyl-(CH₂)—C(O)—NH-fffky-C(O)—NH(CH₂)₂SH;
NBD-(CH₂)₂—C(O)—NH-fffky-C(O)—NH(CH₂)₂SH;
Naphthyl-(CH₂)—C(O)—NH—FFGKY—C(O)—NH(CH₂)₂SH;
NBD-(CH₂)₂—C(O)—NH—FFGKY—C(O)—NH(CH₂)₂SH;
Naphthyl-(CH₂)—C(O)—NH-ffgky-C(O)—NH(CH₂)₂SH;
NBD-(CH₂)₂—C(O)—NH-ffgky-C(O)—NH(CH₂)₂SH;
Naphthyl-(CH₂)—C(O)—NH—FFGK—C(O)—NH(CH₂)₂SH;
NBD-(CH₂)₂—C(O)—NH—FFGK—C(O)—NH(CH₂)₂SH;
Naphthyl-(CH₂)—C(O)—NH-ffgk-C(O)—NH(CH₂)₂SH;
NBD-(CH₂)₂—C(O)—NH-ffgk-C(O)—NH(CH₂)₂SH;
Naphthyl-(CH₂)—C(O)—NH—FFGKF—C(O)—NH(CH₂)₂SH;
NBD-(CH₂)₂—C(O)—NH—FFGKF—C(O)—NH(CH₂)₂SH;
Naphthyl-(CH₂)—C(O)—NH-ffgkf-C(O)—NH(CH₂)₂SH;
NBD-(CH₂)₂—C(O)—NH-ffgkf-C(O)—NH(CH₂)₂SH;
Naphthyl-(CH₂)—C(O)—NH—FFK(Dmt)-C(O)—NH(CH₂)₂SH;
NBD-(CH₂)₂—C(O)—NH—FFK(Dmt)-C(O)—NH(CH₂)₂SH;
Naphthyl-(CH₂)—C(O)—NH-ffk(dmt)-C(O)—NH(CH₂)₂SH;
NBD-(CH₂)₂—C(O)—NH-ffk(dmt)-C(O)—NH(CH₂)₂SH;
Naphthyl-(CH₂)—C(O)—NH—FFFK(Dmt)-C(O)—NH(CH₂)₂SH;
NBD-(CH₂)₂—C(O)—NH—FFFK(Dmt)-C(O)—NH(CH₂)₂SH;
Naphthyl-(CH₂)—C(O)—NH-fffk(dmt)-C(O)—NH(CH₂)₂SH;
NBD-(CH₂)₂—C(O)—NH-fffk(dmt)-C(O)—NH(CH₂)₂SH;
Naphthyl-(CH₂)—C(O)—NH—FFGK(Dmt)-C(O)—NH(CH₂)₂SH;
NBD-(CH₂)₂—C(O)—NH—FFGK(Dmt)-C(O)—NH(CH₂)₂SH;
Naphthyl-(CH₂)—C(O)—NH-ffgk(dmt)-C(O)—NH(CH₂)₂SH;
NBD-(CH₂)₂—C(O)—NH-ffgk(dmt)-C(O)—NH(CH₂)₂SH;
Naphthyl-(CH₂)—C(O)—NH—FFCY—C(O)—NH(CH₂)₂SH;
NBD-(CH₂)₂—C(O)—NH—FFCY—C(O)—NH(CH₂)₂SH;
Naphthyl-(CH₂)—C(O)—NH-ffcy-C(O)—NH(CH₂)₂SH;
NBD-(CH₂)₂—C(O)—NH-ffcy-C(O)—NH(CH₂)₂SH;
Naphthyl-(CH₂)—C(O)—NH—FFFCY—C(O)—NH(CH₂)₂SH;
NBD-(CH₂)₂—C(O)—NH—FFFCY—C(O)—NH(CH₂)₂SH;
Naphthyl-(CH₂)—C(O)—NH-fffcy-C(O)—NH(CH₂)₂SH;
NBD-(CH₂)₂—C(O)—NH-fffcy-C(O)—NH(CH₂)₂SH;
Naphthyl-(CH₂)—C(O)—NH—FFGCY—C(O)—NH(CH₂)₂SH;
NBD-(CH₂)₂—C(O)—NH—FFGCY—C(O)—NH(CH₂)₂SH;
Naphthyl-(CH₂)—C(O)—NH-ffgcy-C(O)—NH(CH₂)₂SH;
NBD-(CH₂)₂—C(O)—NH-ffgcy-C(O)—NH(CH₂)₂SH;
Naphthyl-(CH₂)—C(O)—NH—FFGC—C(O)—NH(CH₂)₂SH;
NBD-(CH₂)₂—C(O)—NH—FFGC—C(O)—NH(CH₂)₂SH;
Naphthyl-(CH₂)—C(O)—NH-ffgc-C(O)—NH(CH₂)₂SH;
NBD-(CH₂)₂—C(O)—NH-ffgc-C(O)—NH(CH₂)₂SH;
Naphthyl-(CH₂)—C(O)—NH—FFGCF—C(O)—NH(CH₂)₂SH;
NBD-(CH₂)₂—C(O)—NH—FFGCF—C(O)—NH(CH₂)₂SH;
Naphthyl-(CH₂)—C(O)—NH-ffgcf-C(O)—NH(CH₂)₂SH;
NBD-(CH₂)₂—C(O)—NH-ffgcf-C(O)—NH(CH₂)₂SH;
Naphthyl-(CH₂)—C(O)—NH—FFC(Dmt)-C(O)—NH(CH₂)₂SH;
NBD-(CH₂)₂—C(O)—NH—FFC(Dmt)-C(O)—NH(CH₂)₂SH;
Naphthyl-(CH₂)—C(O)—NH-ffc(dmt)-C(O)—NH(CH₂)₂SH;
NBD-(CH₂)₂—C(O)—NH-ffc(dmt)-C(O)—NH(CH₂)₂SH;
Naphthyl-(CH₂)—C(O)—NH—FFFC(Dmt)-C(O)—NH(CH₂)₂SH;
NBD-(CH₂)₂—C(O)—NH—FFFC(Dmt)-C(O)—NH(CH₂)₂SH;
Naphthyl-(CH₂)—C(O)—NH-fffc(dmt)-C(O)—NH(CH₂)₂SH;
NBD-(CH₂)₂—C(O)—NH-fffc(dmt)-C(O)—NH(CH₂)₂SH;
Naphthyl-(CH₂)—C(O)—NH—FFGC(Dmt)-C(O)—NH(CH₂)₂SH;
NBD-(CH₂)₂—C(O)—NH—FFGC(Dmt)-C(O)—NH(CH₂)₂SH;
Naphthyl-(CH₂)—C(O)—NH-ffgc(dmt)-C(O)—NH(CH₂)₂SH;
NBD-(CH₂)₂—C(O)—NH-ffgc(dmt)-C(O)—NH(CH₂)₂SH;
Naphthyl-CH₂C(O)—NH—FFK(ε-Z₂)—C(O)—NH(CH₂)₂SH;
Naphthyl-CH₂C(O)—NH-ffk(ε-Z₂)—C(O)—NH(CH₂)₂SH;
Naphthyl-CH₂C(O)—NH—FFK(ε-Z₂)Y—C(O)—NH(CH₂)₂SH;
Naphthyl-CH₂C(O)—NH-ffk(ε-Z₂)y-C(O)—NH(CH₂)₂SH;
Naphthyl-CH₂C(O)—NH—FFFK(ε-Z₂)Y—C(O)—NH(CH₂)₂SH;
Naphthyl-CH₂C(O)—NH-fffk(ε-Z₂)y-C(O)—NH(CH₂)₂SH;
Naphthyl-CH₂C(O)—NH—FFGK(ε-Z₂)Y—C(O)—NH(CH₂)₂SH;
Naphthyl-CH₂C(O)—NH-ffgk(ε-Z₂)y-C(O)—NH(CH₂)₂SH;
Naphthyl-CH₂C(O)—NH—FFGK(ε-Z₂)—C(O)—NH(CH₂)₂SH;
Naphthyl-CH₂C(O)—NH-ffgk(ε-Z₂)—C(O)—NH(CH₂)₂SH;
Naphthyl-CH₂C(O)—NH—FFGK(ε-Z₂)F—C(O)—NH(CH₂)₂SH;
Naphthyl-CH₂C(O)—NH-ffgk(ε-Z₂)f-C(O)—NH(CH₂)₂SH;
Naphthyl-CH₂C(O)—NH—FFK(ε-Z₂)(Dmt)-C(O)—NH(CH₂)₂SH;
Naphthyl-CH₂C(O)—NH-ffk(ε-Z₂)(dmt)-C(O)—NH(CH₂)₂SH;
Naphthyl-CH₂C(O)—NH—FFFK(ε-Z₂)(Dmt)-C(O)—NH(CH₂)₂SH;
Naphthyl-CH₂C(O)—NH-fffk(ε-Z₂)(dmt)-C(O)—NH(CH₂)₂SH;
Naphthyl-CH₂C(O)—NH—FFGK(ε-Z₂)(Dmt)-C(O)—NH(CH₂)₂SH;
Naphthyl-CH₂C(O)—NH-ffgk(ε-Z₂)(dmt)-C(O)—NH(CH₂)₂SH;
NBD-(CH₂)₂C(O)—NH—FFK(ε-Z₂)—C(O)—NH(CH₂)₂SH;
NBD-(CH₂)₂C(O)—NH-ffk(ε-Z₂)—C(O)—NH(CH₂)₂SH;
NBD-(CH₂)₂C(O)—NH—FFK(ε-Z₂)Y—C(O)—NH(CH₂)₂SH;
NBD-(CH₂)₂C(O)—NH-ffk(ε-Z₂)y-C(O)—NH(CH₂)₂SH;
NBD-(CH₂)₂C(O)—NH—FFFK(ε-Z₂)Y—C(O)—NH(CH₂)₂SH;
NBD-(CH₂)₂C(O)—NH-fffk(ε-Z₂)y-C(O)—NH(CH₂)₂SH;
NBD-(CH₂)₂C(O)—NH—FFGK(ε-Z₂)Y—C(O)—NH(CH₂)₂SH;
NBD-(CH₂)₂C(O)—NH-ffgk(ε-Z₂)y-C(O)—NH(CH₂)₂SH;
NBD-(CH₂)₂C(O)—NH—FFGK(ε-Z₂)—C(O)—NH(CH₂)₂SH;
NBD-(CH₂)₂C(O)—NH-ffgk(ε-Z₂)—C(O)—NH(CH₂)₂SH;
NBD-(CH₂)₂C(O)—NH—FFGK(ε-Z₂)F—C(O)—NH(CH₂)₂SH;
NBD-(CH₂)₂C(O)—NH-ffgk(ε-Z₂)f-C(O)—NH(CH₂)₂SH;
NBD-(CH₂)₂C(O)—NH—FFK(ε-Z₂)(Dmt)-C(O)—NH(CH₂)₂SH;
NBD-(CH₂)₂C(O)—NH-ffk(ε-Z₂)(dmt)-C(O)—NH(CH₂)₂SH;
NBD-(CH₂)₂C(O)—NH—FFFK(ε-Z₂)(Dmt)-C(O)—NH(CH₂)₂SH;
NBD-(CH₂)₂C(O)—NH-fffk(ε-Z₂)(dmt)-C(O)—NH(CH₂)₂SH;
NBD-(CH₂)₂C(O)—NH—FFGK(ε-Z₂)(Dmt)-C(O)—NH(CH₂)₂SH;
NBD-(CH₂)₂C(O)—NH-ffgk(ε-Z₂)(dmt)-C(O)—NH(CH₂)₂SH;
Naphthyl-(CH₂)—C(O)—NH—FF—C(O)—NH(CH₂)₃NH(CH₂)₂SH;
NBD-(CH₂)₂—C(O)—NH—FF—C(O)—NH(CH₂)₃NH(CH₂)₂SH;
Naphthyl-(CH₂)—C(O)—NH-ff-C(O)—NH(CH₂)₃NH(CH₂)₂SH;
NBD-(CH₂)₂—C(O)—NH-ff-C(O)—NH(CH₂)₃NH(CH₂)₂SH;
Naphthyl-(CH₂)—C(O)—NH—FFK—C(O)—NH(CH₂)₃NH(CH₂)₂SH;
NBD-(CH₂)₂—C(O)—NH—FFK—C(O)—NH(CH₂)₃NH(CH₂)₂SH;
Naphthyl-(CH₂)—C(O)—NH-ffk—C(O)—NH(CH₂)₃NH(CH₂)₂SH;
NBD-(CH₂)₂—C(O)—NH-ffk—C(O)—NH(CH₂)₃NH(CH₂)₂SH;
Naphthyl-(CH₂)—C(O)—NH—FFKY—C(O)—NH(CH₂)₃NH(CH₂)₂SH;
NBD-(CH₂)₂—C(O)—NH—FFKY—C(O)—NH(CH₂)₃NH(CH₂)₂SH;
Naphthyl-(CH₂)—C(O)—NH-ffky-C(O)—NH(CH₂)₃NH(CH₂)₂SH;
NBD-(CH₂)₂—C(O)—NH-ffky-C(O)—NH(CH₂)₃NH(CH₂)₂SH;
Naphthyl-(CH₂)—C(O)—NH—FFFKY—C(O)—NH(CH₂)₃NH(CH₂)₂SH;
NBD-(CH₂)₂—C(O)—NH—FFFKY—C(O)—NH(CH₂)₃NH(CH₂)₂SH;
Naphthyl-(CH₂)—C(O)—NH-fffky-C(O)—NH(CH₂)₃NH(CH₂)₂SH;
NBD-(CH₂)₂—C(O)—NH-fffky-C(O)—NH(CH₂)₃NH(CH₂)₂SH;
Naphthyl-(CH₂)—C(O)—NH—FFGKY—C(O)—NH(CH₂)₃NH(CH₂)₂SH;
NBD-(CH₂)₂—C(O)—NH—FFGKY—C(O)—NH(CH₂)₃NH(CH₂)₂SH;
Naphthyl-(CH₂)—C(O)—NH-ffgky-C(O)—NH(CH₂)₃NH(CH₂)₂SH;
NBD-(CH₂)₂—C(O)—NH-ffgky-C(O)—NH(CH₂)₃NH(CH₂)₂SH;
Naphthyl-(CH₂)—C(O)—NH—FFGK—C(O)—NH(CH₂)₃NH(CH₂)₂SH;
NBD-(CH₂)₂—C(O)—NH—FFGK—C(O)—NH(CH₂)₃NH(CH₂)₂SH;
Naphthyl-(CH₂)—C(O)—NH-ffgk-C(O)—NH(CH₂)₃NH(CH₂)₂SH;
NBD-(CH₂)₂—C(O)—NH-ffgk-C(O)—NH(CH₂)₃NH(CH₂)₂SH;
Naphthyl-(CH₂)—C(O)—NH—FFGKF—C(O)—NH(CH₂)₃NH(CH₂)₂SH;
NBD-(CH₂)₂—C(O)—NH—FFGKF—C(O)—NH(CH₂)₃NH(CH₂)₂SH;
Naphthyl-(CH₂)—C(O)—NH-ffgkf-C(O)—NH(CH₂)₃NH(CH₂)₂SH;
NBD-(CH₂)₂—C(O)—NH-ffgkf-C(O)—NH(CH₂)₃NH(CH₂)₂SH;
Naphthyl-(CH₂)—C(O)—NH—FFK(Dmt)-C(O)—NH(CH₂)₃NH(CH₂)₂SH;
NBD-(CH₂)₂—C(O)—NH—FFK(Dmt)-C(O)—NH(CH₂)₃NH(CH₂)₂SH;
Naphthyl-(CH₂)—C(O)—NH-ffk(dmt)-C(O)—NH(CH₂)₃NH(CH₂)₂SH;
NBD-(CH₂)₂—C(O)—NH-ffk(dmt)-C(O)—NH(CH₂)₃NH(CH₂)₂SH;
Naphthyl-(CH₂)—C(O)—NH—FFFK(Dmt)-C(O)—NH(CH₂)₃NH(CH₂)₂SH;
NBD-(CH₂)₂—C(O)—NH—FFFK(Dmt)-C(O)—NH(CH₂)₃NH(CH₂)₂SH;
Naphthyl-(CH₂)—C(O)—NH-fffk(dmt)-C(O)—NH(CH₂)₃NH(CH₂)₂SH;
NBD-(CH₂)₂—C(O)—NH-fffk(dmt)-C(O)—NH(CH₂)₃NH(CH₂)₂SH;
Naphthyl-(CH₂)—C(O)—NH—FFGK(Dmt)-C(O)—NH(CH₂)₃NH(CH₂)₂SH;
NBD-(CH₂)₂—C(O)—NH—FFGK(Dmt)-C(O)—NH(CH₂)₃NH(CH₂)₂SH;
Naphthyl-(CH₂)—C(O)—NH-ffgk(dmt)-C(O)—NH(CH₂)₃NH(CH₂)₂SH;
NBD-(CH₂)₂—C(O)—NH-ffgk(dmt)-C(O)—NH(CH₂)₃NH(CH₂)₂SH;
Naphthyl-(CH₂)—C(O)—NH—FFCY—C(O)—NH(CH₂)₃NH(CH₂)₂SH;
NBD-(CH₂)₂—C(O)—NH—FFCY—C(O)—NH(CH₂)₃NH(CH₂)₂SH;
Naphthyl-(CH₂)—C(O)—NH-ffcy-C(O)—NH(CH₂)₃NH(CH₂)₂SH;
NBD-(CH₂)₂—C(O)—NH-ffcy-C(O)—NH(CH₂)₃NH(CH₂)₂SH;
Naphthyl-(CH₂)—C(O)—NH—FFFCY—C(O)—NH(CH₂)₃NH(CH₂)₂SH;
NBD-(CH₂)₂—C(O)—NH—FFFCY—C(O)—NH(CH₂)₃NH(CH₂)₂SH;
Naphthyl-(CH₂)—C(O)—NH-fffcy-C(O)—NH(CH₂)₃NH(CH₂)₂SH;
NBD-(CH₂)₂—C(O)—NH-fffcy-C(O)—NH(CH₂)₃NH(CH₂)₂SH;
Naphthyl-(CH₂)—C(O)—NH—FFGCY—C(O)—NH(CH₂)₃NH(CH₂)₂SH;
NBD-(CH₂)₂—C(O)—NH—FFGCY—C(O)—NH(CH₂)₃NH(CH₂)₂SH;
Naphthyl-(CH₂)—C(O)—NH-ffgcy-C(O)—NH(CH₂)₃NH(CH₂)₂SH;
NBD-(CH₂)₂—C(O)—NH-ffgcy-C(O)—NH(CH₂)₃NH(CH₂)₂SH;
Naphthyl-(CH₂)—C(O)—NH—FFGC—C(O)—NH(CH₂)₃NH(CH₂)₂SH;
NBD-(CH₂)₂—C(O)—NH—FFGC—C(O)—NH(CH₂)₃NH(CH₂)₂SH;
Naphthyl-(CH₂)—C(O)—NH-ffgc-C(O)—NH(CH₂)₃NH(CH₂)₂SH;
NBD-(CH₂)₂—C(O)—NH-ffgc-C(O)—NH(CH₂)₃NH(CH₂)₂SH;
Naphthyl-(CH₂)—C(O)—NH—FFGCF—C(O)—NH(CH₂)₃NH(CH₂)₂SH;
NBD-(CH₂)₂—C(O)—NH—FFGCF—C(O)—NH(CH₂)₃NH(CH₂)₂SH;
Naphthyl-(CH₂)—C(O)—NH-ffgcf-C(O)—NH(CH₂)₃NH(CH₂)₂SH;
NBD-(CH₂)₂—C(O)—NH-ffgcf-C(O)—NH(CH₂)₃NH(CH₂)₂SH;
Naphthyl-(CH₂)—C(O)—NH—FFC(Dmt)-C(O)—NH(CH₂)₃NH(CH₂)₂SH;
NBD-(CH₂)₂—C(O)—NH—FFC(Dmt)-C(O)—NH(CH₂)₃NH(CH₂)₂SH;
Naphthyl-(CH₂)—C(O)—NH-ffc(dmt)-C(O)—NH(CH₂)₃NH(CH₂)₂SH;
NBD-(CH₂)₂—C(O)—NH-ffc(dmt)-C(O)—NH(CH₂)₃NH(CH₂)₂SH;
Naphthyl-(CH₂)—C(O)—NH—FFFC(Dmt)-C(O)—NH(CH₂)₃NH(CH₂)₂SH;
NBD-(CH₂)₂—C(O)—NH—FFFC(Dmt)-C(O)—NH(CH₂)₃NH(CH₂)₂SH;
Naphthyl-(CH₂)—C(O)—NH-fffc(dmt)-C(O)—NH(CH₂)₃NH(CH₂)₂SH;
NBD-(CH₂)₂—C(O)—NH-fffc(dmt)-C(O)—NH(CH₂)₃NH(CH₂)₂SH;
Naphthyl-(CH₂)—C(O)—NH—FFGC(Dmt)-C(O)—NH(CH₂)₃NH(CH₂)₂SH;
NBD-(CH₂)₂—C(O)—NH—FFGC(Dmt)-C(O)—NH(CH₂)₃NH(CH₂)₂SH;
Naphthyl-(CH₂)—C(O)—NH-ffgc(dmt)-C(O)—NH(CH₂)₃NH(CH₂)₂SH;
NBD-(CH₂)₂—C(O)—NH-ffgc(dmt)-C(O)—NH(CH₂)₃NH(CH₂)₂SH;
Naphthyl-CH₂C(O)—NH—FFK(ε-Z₂)—C(O)—NH(CH₂)₃NH(CH₂)₂SH;
Naphthyl-CH₂C(O)—NH-ffk(ε-Z₂)—C(O)—NH(CH₂)₃NH(CH₂)₂SH;
Naphthyl-CH₂C(O)—NH—FFK(ε-Z₂)Y—C(O)—NH(CH₂)₃NH(CH₂)₂SH;
Naphthyl-CH₂C(O)—NH-ffk(ε-Z₂)y-C(O)—NH(CH₂)₃NH(CH₂)₂SH;
Naphthyl-CH₂C(O)—NH—FFFK(ε-Z₂)Y—C(O)—NH(CH₂)₃NH(CH₂)₂SH;
Naphthyl-CH₂C(O)—NH-fffk(ε-Z₂)y-C(O)—NH(CH₂)₃NH(CH₂)₂SH;
Naphthyl-CH₂C(O)—NH—FFGK(ε-Z₂)Y—C(O)—NH(CH₂)₃NH(CH₂)₂SH;
Naphthyl-CH₂C(O)—NH-ffgk(ε-Z₂)y-C(O)—NH(CH₂)₃NH(CH₂)₂SH;
Naphthyl-CH₂C(O)—NH—FFGK(ε-Z₂)—C(O)—NH(CH₂)₃NH(CH₂)₂SH;
Naphthyl-CH₂C(O)—NH-ffgk(ε-Z₂)—C(O)—NH(CH₂)₃NH(CH₂)₂SH;
Naphthyl-CH₂C(O)—NH—FFGK(ε-Z₂)F—C(O)—NH(CH₂)₃NH(CH₂)₂SH;
Naphthyl-CH₂C(O)—NH-ffgk(ε-Z₂)f-C(O)—NH(CH₂)₃NH(CH₂)₂SH;
Naphthyl-CH₂C(O)—NH—FFK(ε-Z₂)(Dmt)-C(O)—NH(CH₂)₃NH(CH₂)₂SH;
Naphthyl-CH₂C(O)—NH-ffk(ε-Z₂)(dmt)-C(O)—NH(CH₂)₃NH(CH₂)₂SH;
Naphthyl-CH₂C(O)—NH—FFFK(ε-Z₂)(Dmt)-C(O)—NH(CH₂)₃NH(CH₂)₂SH;
Naphthyl-CH₂C(O)—NH-fffk(ε-Z₂)(dmt)-C(O)—NH(CH₂)₃NH(CH₂)₂SH;
Naphthyl-CH₂C(O)—NH—FFGK(ε-Z₂)(Dmt)-C(O)—NH(CH₂)₃NH(CH₂)₂SH;
Naphthyl-CH₂C(O)—NH-ffgk(ε-Z₂)(dmt)-C(O)—NH(CH₂)₃NH(CH₂)₂SH;
NBD-(CH₂)₂—C(O)—NH—FFK(ε-Z₂)—C(O)—NH(CH₂)₃NH(CH₂)₂SH;
NBD-(CH₂)₂—C(O)—NH-ffk(ε-Z₂)—C(O)—NH(CH₂)₃NH(CH₂)₂SH;
NBD-(CH₂)₂—C(O)—NH—FFK(ε-Z₂)Y—C(O)—NH(CH₂)₃NH(CH₂)₂SH;
NBD-(CH₂)₂—C(O)—NH-ffk(ε-Z₂)y-C(O)—NH(CH₂)₃NH(CH₂)₂SH;
NBD-(CH₂)₂—C(O)—NH—FFFK(ε-Z₂)Y—C(O)—NH(CH₂)₃NH(CH₂)₂SH;
NBD-(CH₂)₂—C(O)—NH-fffk(ε-Z₂)y-C(O)—NH(CH₂)₃NH(CH₂)₂SH;
NBD-(CH₂)₂—C(O)—NH—FFGK(ε-Z₂)Y—C(O)—NH(CH₂)₃NH(CH₂)₂SH;
NBD-(CH₂)₂—C(O)—NH-ffgk(ε-Z₂)y-C(O)—NH(CH₂)₃NH(CH₂)₂SH;
NBD-(CH₂)₂—C(O)—NH—FFGK(ε-Z₂)—C(O)—NH(CH₂)₃NH(CH₂)₂SH;
NBD-(CH₂)₂—C(O)—NH-ffgk(ε-Z₂)—C(O)—NH(CH₂)₃NH(CH₂)₂SH;
NBD-(CH₂)₂—C(O)—NH—FFGK(ε-Z₂)F—C(O)—NH(CH₂)₃NH(CH₂)₂SH,
NBD-(CH₂)₂—C(O)—NH-ffgk(ε-Z₂)f-C(O)—NH(CH₂)₃NH(CH₂)₂SH;
NBD-(CH₂)₂—C(O)—NH—FFK(ε-Z₂)(Dmt)-C(O)—NH(CH₂)₃NH(CH₂)₂SH;
NBD-(CH₂)₂—C(O)—NH-ffk(ε-Z₂)(dmt)-C(O)—NH(CH₂)₃NH(CH₂)₂SH;
NBD-(CH₂)₂—C(O)—NH—FFFK(ε-Z₂)(Dmt)-C(O)—NH(CH₂)₃NH(CH₂)₂SH;
NBD-(CH₂)₂—C(O)—NH-fffk(ε-Z₂)(dmt)-C(O)—NH(CH₂)₃NH(CH₂)₂SH;
NBD-(CH₂)₂—C(O)—NH—FFGK(ε-Z₂)(Dmt)-C(O)—NH(CH₂)₃NH(CH₂)₂SH;
NBD-(CH₂)₂—C(O)—NH-ffgk(ε-Z₂)(dmt)-C(O)—NH(CH₂)₃NH(CH₂)₂SH;
wherein Dmt is 2,6-dimethyl-L-tyrosine; dmt is 2,6-dimethyl-D-tyrosine; NBD is 4-nitro-2,1,3-benzoxadiazolyl.

46. (canceled)

47. A method of treating a patient having a cancerous condition comprising:
administering a pharmaceutical composition according to claim 24, to a patient having a cancerous condition, wherein said administering is effective to inhibit cancer cell survival.

48. A method of treating a patient having Alzheimer's or Parkinson's disease comprising:
administering a pharmaceutical composition according to claim 25 to a patient having Alzheimer's or Parkinson's disease, wherein said administering is effective to treat symptoms of disease.

49-50. (canceled)

51. A method of imaging a cell, the method comprising:
providing a peptide according to claim 1, wherein Z₁ or Z₂ is a fluorophore, or a composition comprising the peptide;
contacting a cell with the peptide or the composition, whereby the peptide, or micelle structures formed by the peptide, are taken up by the cell and targeted to the Golgi apparatus within the cell; and
obtaining an image of the cell, whereby the Golgi apparatus is identified by fluorescence from the fluorophore.
